SEQUENCE LISTING (13 sequences)

SEQ ID NO: 1 - protein, 1744 aa, Homo sapiens [sequence omitted]

SEQ ID NO: 2 - DNA, 27624 bp, Homo sapiens
tgtcttttggggtttgtttttattctctctttgagttttgtttccttatgcgcccagttacttttgaaaatgttctgggcagatttgcctagattaataaatgccctccatgttccaattacttttttttttttgagacagtgtcttaccctgtcaccaagctggagtgcagtggtatgatcttggctcactgcaacctctgcctcctgagttcaagtgattctcctgcctcagcctcccaagtagctggcattacaggcacctgacaccacgcccagctaattttttttttttttttttttttgagacggagtctcgctctgtcacccaggctggagttcagtggcatgatcttggcttactgcaagctctgcctcctgggttcacccattctcccgcctcagcctcccgagtagctgggactacaggtgcccgccactatgcctggctaattgtttttttttttgtatttttagtagagatggggtttcaccgtgttagccaggatggtcttgatctccggacctcgtgatccacccgtctcagcctgccaaagtgctgggattacaggcatgagccaccgcatctggcctatttttgtatttttaatggagaccgggtttcatcatgttggccaggctggtcttgaacttgaacttctgacctcaagtgatccacccttagcgtcccaaagtgctgggattacaggcatgagccaccgtgcccggccccagttatttttatttttattttttgagttagagtctcactctgtcacccaggctggagcgcagtggcatgatctcggctcacagcaactttctgggttcaagcagttctcctgtgtcagcctcctgagtagctgggactacaggcacacatcaccacgcccggctaatttttgtagttttagtagagacggggttttaccatattggtcaggctgatattgaactcctgacctcaggtgatccacccacgtcagcctcccaaagtgccgggattacaggcttgagccatctcgcccggcctacttagatgttatattagtggtaattcctgttatcctgtgagctctttagtgtctaaacaattttttttaagagatggggtctcactgtgttgcccagttgcaatcatatcttactgcagcctcaaactcctgggtcaagtgatcctcttgccttagtctcccaagtagctaggaccataggtgtctgcccccacgcctggctgtttttacattttttgtagagatgtggcgggtgggggggtctcactgtgttgcccagactggtctcgaactcctgtcctcaattgatcctgctacctcagcctcccaaaatgctgaattacaggcatgagccactgtacctggtcttaaacaattttaaaataacatttttatccaggattttagttaattttcaacaggtggattagttcttgctgtattctcgtaaacagaagtcctggtttatttttatttgttttaaacattgaatcccatactcctccccaccttaccctacccagaatttagactgttaatgttttgaagccacagcctgcatcttaatcactattttatcttagtgcctggtcttagaaattatattgactctttgatagaccatatataaggcaggtggatgagaatgtgggtagctagttggaaaaggctgcttggtcatttgcttgattattttctcacacagtttttcctttactaagagaaaatgcccccatattggcaaacaaaatctccctgcctgagagcgcccagagtatagcagagcatcttaccctgatacgcctcttttcactctcttctctgtggagacagaaggagcttcaagagcagggggagatcagaatcgtccagctgggcttcgacttggatgcccatggaattatcttcactgaggactacaggaccagagtatgtgactgtgtgcgtcaggggtgctggggggagggcacaggttgggggagacagggaacttgggaaacagaaataaaaacaaaagaaagaatttccctgcccccacatcccatggagagggcacagggccctggtaaatagtaatatgagggagagagacaggagggaaagagggaggagtgagagggtaaagagggggggagaggagggggaggaggaggaaggaaggagggggaggaggagggggggaggaagagggggaggaggatgaagaggaggaggaagaagaagggtatgagaggtggaaggatctgagcaagaggtaagacaggaagagaaatgctgtcctgggggtggaggttggtagagagtgagggtggggatggaccatgtctctcatctctgcttgtaggtcctcaaggcctgtgatggccgaccgtatgctggggcagtgcagaaatttctagcttcagtacttccagcctgtggggaccttagtttccagcaggaccaaatgacacagacctttggcttcagggactcagaaatcacgtgagacttgtggaaccaaccaaagtcaggcatctggtgcttccctgcctccctccagttccatccagcctgtcctcctgtttttttggtgaacctgccagaaaagctgccaaaaagctgactcttcttgttaataaaatgacccaagtttgtattcctccccacaagagaggaggcctatcttacctgggccttagaaagagccctgaaatagaattcagttcttggtggcttatcaaaagcacacaggggcctggcaggaagtgtaaaagcttgatgttaatcatactgggactaagaggatagagaatggtaggagctgggatacccctaaacattcacattaaaacaaaaaaaacccaaagctaaaaaacaactgggcaggagctaaataaaaatctaattttgagaggctgtatctggctcaggcctcctactttgtaacccatggaatatgtgaaagcatttgaaaaactatagcactgatctcacatgggcagacacactctcagagagatgtggtgggagccatggcgcagtctgcctaggcagtggcaggagcgcagaagactctgattcctctcctcggtcctaagaccgaatgtgtgtcaggacatgtggtcagggaagagaagctatttaactgaaccagtaatagtagcaggaaaagaaaaagtggagggagggcagtccaggtagggggcctggaacaagcaactgcaccaacagaggcagttggtgcgagcacagaaccaccccaggctgggattttgttatccagtctctcttgcatggttgcccgtgtttctggagacttgtgtaaacattaatggatgaggaggagagatggttctcagagcccagccctcatctctgctggcttcccactgccctcaggcatctggtgaatgctggagtcctcaccgtccgagatgctgggagctggtggctagctgtgcctggagctgggagattcatcaagtactttgttaaaggtatcccatctgcagctcaagcctgcagcccctcaccttttggtggctcctcaggcctctaggccttattcacctttcccctttcctgtgccacttctcctctagggcgccaggctgtccttagcatggtccggaaggcaaagtaccgggaactgctcctatcagagctcctgggccggcgggcgcctgtcgtggtgcggcttggcctcacctaccatgtgcacgacctcattggggcccagctagtggactggtgagtctttccctggcctctggcagattatggagcaatgacccaaagtgggatttcctcccagctcatgcttagtttcctagtgaaggccagtggctctcattcttctctggaacccgggagcaccccttcccaagttctaagttctcctcacagcttgagcctaggcgtctggctccagccttgtctttctcctgcacagcatctctaccacttcaggaaccctcctccgcctgccagagacatgaagattctgctcatcattgctcagctcctcagagtgggccgggaggggactagaagagctgcatgatggtggctgagacagggtcaccttgggaaggcttgggagccaggatgagtgtcgggctctcgtgtgtgcaaaaggtcagatgtgactgctgctgtttgcctggtttctgacccagtggtggggtttgagcaatgcttctctgcccttccatggaaagtggaaccagaaatggtgccaaggctgtggctgttccctttcgtgtaaaatggtgctgttattactctgtcttgaaataggaaggtgggatttctggggaggctggtgaaggagggcagggttcttttctctacgtgtcatgttaaaattgccaaataaagtacctctgcctgtgatattttctggatgtcctttatttactgtgacgtgtgtttgggtgccttgtttaggggtagaggtgaagtctgagctttgcctcattcagagaggaaaggggtcaggggttcactctgacgttcaggccattctccctgtggagtggtgagggtgtacctaatctcctaaaccacggaatttctgttagggcctaaaaaagcaaaagcctagtatagttcaatttgtgttggaatgaaagtaagagacaagtgtcttagaagcctgtcattgttttgtgagggcctttaaatatcctgtactcgtgggccatgttgggcccttgtacgcccaggtatacatgagcttgtgtgcacctataccctgatacagatatacctggtagggggaggtgctcaggcactggaatgagaggagttaacggggaaggacagggttatttctgggccaagattcagagtttcccatggacacccaggtgtccggggtgcccccacaactctgggcctgaggccagttgcacttcttggctgtcacgtggtttcccagcttagctgggctgggggaggagcaaggtccagagtcaactctgccccgaggcctagcttggccagaaggtagcagacagacagacggatctaacctctcttggatcctccagccatgaggctgctctgggggctgatctgggcatccagcttcttcaccttatctctgcagaagcccaggtcctggaggcgggatgctgggtgcttggattggggcagggctggcatcgggacccgattcaggagtgagggagagcaggggtggaggtgtcagagcgaagtctgactgctgatcctgtctgttctccccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtaaccagaccccatgccctcctgctgcttgtgggggcctcctgccctgttcccatctgtcttgtaagtgtcatcatcttcccactggcctcctcccctcctgtcttcccaccctggcattctccttccacgtttctcccttggtctctgtcctttttggtcagctgtctcttgctctgtgacccgctccctctccctctccctctcctgacaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcggggtgagtctcagccccagggcctcaacctttaaccccctccgagccctctcaggatgagtttggtgccccctaagtgagataacctgaaagaaagtgccacacagaaggggtgcttaggaaacatttgtcccctgctccctctgtggagtttgacccaccctccccttgcacatggacccctgctcacctctctcctcctccactcccagttcggtaccgggtctttgctctggatcagaagatgcgcccgagcactgacaccatcacagtcatggtggaggtgagtccccgacctctggccttcctgatcctggccactgatgtgacctcctgcctgtgagcacttctccccttgcagaactctcacggcctccgcgtgcggaagaaggaggtgtacatgccctcgtccatcttccaggatgactttgtgatcccagacatctcagagtgagcgctcccaatgtgggggctgcccccaagctacaccaccccaattcctgttaggctctccacctcccacacagaggcacgtccccagatgccctgaccctcagcctcctgagcctctggttaacccccacagtcctcttcccagggaagcaggctgctggctctccgtgccccactgtacagatgggctgagccccttccttgtccattctcaggccagggacctggaagatctcagcccgattctcagatggcctggaatccaacagcagcacccagtttgaggtgaagaaatatggtgagagctggaaactggagggacaggcagctgctttcctgaaggaaataagggtggaaggagaggtactgggagcagctcagggcagggagatatgggtgccacagccctgagcagaggggagtctttgagctggagtctgacctgcctatcccttcaccctgggtcagtccttcccaactttgaggtgaagatcacccctggaaagccctacatcctgacggtgccaggccatcttgatgaaatgcagttagacatccaggccaggtaatacctccctccccacctctgcccaccagcaccgggtcctgctccctactcagtatgaatgggctcctgcttccctgccctcgggccattattccccccagcccttggcccaccctcttctctctgccacgacaggtacatctatgggaagccagtgcagggggtggcatatgtgcgctttgggctcctagatgaggatggtaagaagactttctttcgggggctggagagtcagaccaaggtaggaaggagaataggggctggggaggggaaggggcaagggaggtgaggtgggagactcagtctcaccctatgtcctgtttctttctatgccccagctggtgaatggacagagccacatttccctctcaaaggcagagttccaggacgccctggagaagctgaatatgggcattactgacctccaggggctgcgcctctacgttgctgcagccatcattgagtctccaggtgggtgactttcccttattgtaaccccagacccttgcctctgacctctgagctaaccctctgtcctccggcaccaacaccaccccacttctcacatctcatctcagactcaaaaccaggaaacacccaggagacctggtttctctccaactctgtctctgtgactcggcccttttccctggctgagtttatttatttctttgctcgttctgctcattccttcactcctccagtggacatgtgttgttcaatgccccgtgctaggcctcagcatgcacagacatgttggggaccagcctcaacgccacccgtagggttcctgaagtccattggtgacacaggaatgagaagagacaggttaagagttcataaagagtgggggccagggggccaattgcaaaatggaggctgcaaaaggctcagagctctggtctccacactattttttgagtacagtcactcagatctaagaagcagatgttcagggagaaacagtgaaagggaggcagtgggtcataggcgtaatctatagcaatagagttttaaatgaatctcctttgtgctcaaacagcatgtctttaaattatcggagagtagctggtggaagtgggcttagctagaagactgcatgtctgtccaatgcttcaaaggagggtctttctccttgaacagagtgtttacagataagacagggggtctcactctgagcatgggaacatgatggcaattaggaggcttttcttctcagaggcctcttgtggctttccacaacttattgtctcatatttttatggacagtttatacaggcaccccacaagtccttttcccaacatgcccccctccctttttttttttttaaccgctattgctattatggcttatttgtggtgtttggtctgttttcagaagtgtcttttgcatctgtagactaaaagtaaacagcataaacagatacacattaaagtaaaatttgtaatagttgatcctttaatggtcttaatctgtttaagaggatttatgtttgaaagtccgtcagtagctccaatgagaatgtcagtctcaggcaggagggttaaatgagcctgagatgctttaaaaacctgtttttttaaaatttggttatatttaatgttaaatttttatttttttcttttagatgatgtctaactttttaaaaatgatgtttagtagtattatacgaatggggagttatgtagaaattggaagtatttcaattacattgtacttctaattgatgttttaagtttattgtacgatcttccatttaaataacagtctgtctaagatcatttgtttgatttgtcaattgttggtctatttgggtctgagaattccacaattttgaggaattttttgttaactatttatatattttgtagtttgaacagaggagtgtaaagcaattccagcagccgcagcagtagctgtgactgcaataaggcccataagactgttataagggtaaaaataaatctctttgttttggtaaacacttttttttaaaacatttttgtgacaatatgaatggaaggagaggctttctaaggtctattgagggaaaccagtatccaaactcctttcttagtttttatcagtaacacagatgtttttacaccgaacgtggaattaatacaggtgaaaaggtgacagttttgacaagtaatagtttgagaattaggtcgaatgtcaatatttttgaccattaacataaaaggagggttgacacaactctgaatgggcactgttttgttggaagaaaactgatacgcaaattgaagtttttaaccttttttttttaaagataatatatttttttctaaacttaaatatgagattgggccattattaactttcataatttggagtgtttagggcctattattggattaattattttgggatgtgggccagctgtactaaaattggtccaaattatgggaaaatgagcacgtttttcagtgtaagtagtgttacctttttgatagtatagtttctgttttagttttgtcttgtatttattattttgatgggtacaattaactgtaaaggtcccctcaggggaccaattaatgacaatttcataggaattattttgtagtaccatagtgtgatcagagatgtaattttttttaattaatatttttaaattatttgaccattgttaaggttgttggcacctcttttttgggggcttaaactgttaattgaattgaactctgtgaatgatccgggctccatccagaaaataaatgataggatactggtctttgattatgacctggaattttaactagtcaatgttgtcggtagccttttaggcaaccgatagttggccttatgtaaagaggggggaactgataacctatggacacatttattaacttttttttttttcctttgggtgagagggcccatgagtatttgtaggcttagggatccaaacgctattattaacataaacttcaactgggggttttaaccatgtgacaggcctaattaaaggcaggaatgggacacatgcccaataggtataattttgggctgttgtagccacaggtttgttaggcgaggaggtcactgtttttattttggctttgtattctaggattagtaaataacagaagacaaacatgagtataattagtaacttttttttttagtaaaagagtgacctgtagtgttacttggcatcttagtttactatatgttattaatgaggaaccccactgggggtatgttaatttattctagctaagcagttatgttattagaagctgagaagggggtgtttgttaaagtaacagggcagaagaaaggcggatttaagatacgagcttaatacagtgtagcaggtataggtagtaggcaaagtgagagaattaaaaatgaataaattatttggcttagacttttgtttttttagtataatgtctgaggcctgtgttgtttgtggaagtcgcattgttgaggctgtagttcctgtagggtcttttttaggctggttcaaatgtttttttattttttaattttttatcctttgatgaggatgtagtctttaggctggtactggaaattttaggagtggcgtctgtgttaagagactttttacaatttttaaagagcaggttagtgttttaagaaaaacttgtgttttattttaatgtttagtttatagaaaactggatgatatctttttaactttagtaaatacgtttacacacggaattttttacaattatcattttaaaacttgtttagatctttaaaacaaaattaaacaaccttttttgtataaattttttataactttttttatgacttttacagacaatttttaacatgtcttaactttttatgttttataatttttttactaaaggtacatttttataactttttaaatttttttacttttttgtatttttttgatttttgtcttagtcttttttttacttttatttttttaaatgtgtaataattagatgagtgttggtaacaatggatgtatgtacatattttagtttttaaaatttagggatgtgtttaacatctgtttgccagaactgactaggttccaattctttacggttaacacctattgaaggagggtatgtgcctgtgagctggtaatctgggcattgtgggataatttgtttagccagcctctgtgtaagttgaaattatttagataagtttctccaattttggtggaataatcgatgtgattgggtggcttggtcaagcagtgatgtcataacctgaaggtctgcttgattattgccgtaagccaatgggccaggcagagagctgtgggctcgaatgtgtgtaataaaagtaggatgtgtaccttggtctagtaattgttgaagttgaagaaaaagaccacacagagtgggctccagagcaaacttaaggctgtaatagtttttaaataaatacacagaataaccttagctctctgaatgttagtaaattcagatcaagtgattggattatgtggtctccaccagactgttgctttttcatgtttaccagacccaccagtaaaaacagctatggctccttccaaaggggcatcacaagtaatttttggaagaacctatgtagttaattttaagaattgaaaagtttttaggataatgattattaatacatccaacaaattttgttaaattaatctgtcatgtaactgagttaataaatgcctgtttaacctgatttttatttattggaactataatttttattgggctcagtgccacaaaatttaataattcatatatgagcctgtccaattagaattgccatctgatttaagtatactgtaagtgcttttatggtattatgtggcaaaaaggaccatttaactaaatcatcattttgaacaataacccccattattgtgtggttagtgtgaagtagggaacacaatgaattataaaggcaagtctgagtcaatcctactgacctgggcttgctgaattttgttttcaattactgataactctttcatggcctcgggtgttagttctctgttactgcgtaagttggtatttcccctcaatattgagaagagattagacatagcataagtaggaattgctaaattgggccaaatccaattaatatcttctaacaatttttgaaaattatttaaggttttgaaagaatctcttctaatttgaaccttttgaggcttaatggctctatcctgtacttgtattttcaaatactgaaaaggagtggttgtttgaattttgtcaggtgctataagtaattcagcatttgtaattgtcttttgcaaagattaataatattgaataagttggtctctactttttgctgcacaaatctggaaactgatctctaacaggctggatagttctgcctacaaaagtttgacaaactgtgggactatttaacataccctggggcaaaactttccaatgatatttggctgcaggttttttgttattaacggcaggaatggtaaaggcaaattttttgaaatctgcctctgctaaaggaattgtaaaaaagcagtcttttaaatctataataacaagcggtcagtctttagggagcacagtgggggatgggagcccaggttgtaaggctcccatcggttgaattacagcgttgacgccatctaccggactttttcttaattacaaatactggggaattccaaggagagaaagtgggtgaaatatatcctttttttagtagtttattttataaagcacccccaacttttccttagggagcggccactgttcaacccagacggggcgccgggtcatccattttaagggaaattgctccttcactgtaataactgtagggtgaacctgaattgccccatctccataatgaactgtgggtcgggcaataatgggcacggtgagccaagtctcgggctccctccccctgcacccactcggctgaggaggaggtggccattctggacatttctctacaggaaccgtgggctgaacaattttttgagtaggtttagggagactggggagattggcataaatcatcttcagactctcctttttgttagtactcggtagaggtggttcagagttctgattatcaaactcctctctctcctcctctgactcagcctcattatctgtctgaaaaggctccagtgctgcatgcaccaatgaccaaagcgaccaaacaggcaaaggaatttcctttccttctctatatgctcttttaaggtcctttccaactccttcttaatgttttaatttcaaagtttcctgttttgggaaccaagggcaaaattgttccatagcatgaaacaaatccataagattttccgtatcaacttttaccccaccatgcatgcttgaagagctgccgtaggaagctcaaatacgtggtgtacttactttcagtttttcccattgtgtccctagctttctctgggcgccccgcttacctgtagaggttaaaacttttatgtccttgggagtcctttgttcgttggtcctctgtttcacatgcttgagcgtttcctcaccagattcttttgggccccacgttgggcgccagaatgttggggaccagcctcaacaccacctgtagggtacctgaagtctggtggtgacaaaggaatgagaagagacaggttaagagttcataaagagtggaggccagggggccaattgcaaaatggaggctgcaaaaggctcagagctctggtctccacactatttattgagtacaataacttagatctaagaagcagatgttcagggcaaaacagtgaaagggtagcagtgcgtcacaggcataatctacagcagaagcgctttaaatgaatctcctttgtgctcaaacagcatatctttaacttatcggagagtagctagtgggagtgggcttaactaggagcctgcacgtctgtccacattccaatgcttcaaaggagggtctttctccttgaatacagtgtttacagataagagagagcaggtctcgctctgagcatggcaattaggaggcttttctcctcagaggcctcttgtggctttccacaacttattgtcccatatttttatggccagtttatacaggcaccccacaagtccttttcccaacacagacaggaatacggcagcctgtgccctgggagctcactgtcttgtgggagggaaccactcaagccactccccacttgtcctcctgtccctctcttcttgggctctgtcccccacctctctctgtcctttgtcttgcaggtggggagatggaggaggcagagctcacatcctggtattttgtgtcatctcccttctccttggatcttagcaagaccaagcgacaccttgtgcctggggcccccttcctgctgcaggtttcttccagaggggaaggatgagtagggaggatgtggtagttaggagggctcagggtctgaccactctcttttgcctgccctcctttacctgcctaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtaggactcctcggacccctgggagatggtgggggaaggggaggagggtgagctggggtcccaaggatccatggcctgacttggggggaaggtggggtacttggctctgagctactaccctattcgcacctgaccccctctccaggtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatggtgtgcatgagctggggagtcacggagggctggggtgcagggaagagccctctgggtggggctgggggggttcaaggctgaggctgtcccatgaagaggcaaccactcttgtccctcccattcttggcccagatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaaggtgaccggggtcaggagagatggcacttgtgccgagggggttgaggacagggtgattgccaacagggcatggatttagcttgggggcagtgaggataccgggactgaaggaagctctcccactctgaccgcccccacctgccgcccctgccagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtttgtccagaccctctccacagctctctcacccctccatggctcatccccctgcttccctgagccttgggcgcagcccctggatcccactgaggctccccacagtctcttccccacttggccctgtggtctccatctcctggctctgtatcctttcctatccccccatgtgctgccctctcacctgtgccgagtgctcagtcctgcccctcagccacacttggctcctagcattcctgcctttcttgcaggtctttgaagctatgaacagctatgacctcggctgtggtcctgggggtggggacagtgcccttcaggtgttccaggcagcgggcctggccttttctgatggagaccagtggaccttatccagaaagagtgagaacagagaaggaaggggagtgggtggcgggaagataaggaaggaggaagggcctgaggggaccagctggaagagtccgggcaggaagggctgggcaggggaaggggaggaggggaggaggccgagtgcctgacggctggactgcagcctttctctctaccaggactaagctgtcccaaggagaagacaacccggaaaaagagaaacgtgaacttccaaaaggcgattaatgagaaatgtgagttgcgggtgcctaggcagtagcttgggctctccacctgggatccgggttgggggtctgcctctctgcccctcggctccttgctgaacccacgtgtggtatttggggccagagatccgaattccgggattacgagtggaaggtgggcagctctctccagcagcctctcttatgttgctggtctcaaggggtcggggcgggggctgaggtgtatgtcctttttgtcctctcatgctcacccccacctggccctgcagtgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgaggtgaggggctgggtggggctagggcacaggtggcggcgcttggaaaggcagaacggtcccctcctcactcccgtccaccgtggtcccccagccctggagatcctgcaggaggaggacctgattgatgaggatgacattcccgtgcgcagcttcttcccagagaactggctctggagagtggaaacagtggaccgctttcaaatgtgagagtgtgtgccggcccggccttttctctgtgctgtgtctcggggccagccggggtagacgggccttctctgcctttccctacacagattgacactgtggctccccgactctctgaccacgtgggagatccatggcctgagcctgtccaaaaccaaaggtgatgtcaccctgtctgggcctcaggtgaccctgcttccatttccctgtaccccagctccctgttccctttgctcttagtgtaggaagagggtccagtgatctggggaggtctgtgccagcgtgcagctggcgtgggccagagggcagaggcggactgagacagagctgggtcacccccacccctccctcctgtggccctgaagctttgatggcccctctgatctctgcccctgtgcccacgcttcctttccctcaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgaggccccataggagcctgagcatacaggagttgggggagccagggcccagtgaggggtggggaggctaaccgggccaggactctggccatcctcgttttcctgccctcaggtgagcgtccacgtgtccccagtggaggggctgtgcctggctgggggcggagggctggcccagcaggtgctggtgcctgcgggctctgcccggcctgttgccttctctgtggtgcccacggcagccgccgctgtgtctctgaaggtggtggctcgagggtccttcgaattccctgtgggagatgcggtgtccaaggttctgcagattgaggtgaatggagcacccctgaatataagtccccgggcccccagctttgtcctccaccctcagcactctctctgctggccaggccaggggcccaacacccaaaccaatgccttggtctgttcccatcttctacaattctgatccaactctgtccctggagttgaaactcaaagttctgggggagtctgcgctagcagggcaggctgtagtcctgtgtgacctcacaaccatgttttccctgagacagaaggaaggggccatccatagagaggagctggtctatgaactcaaccccttgggtgagtgaccctctacctccagccattggtttcctaagtgggtacaggtggtgggggatgtggacagcaggacaggctgccaacttcccccatttccccagaccaccgaggccggaccttggaaatacctggcaactctgatcccaatatgatccctgatggggactttaacagctacgtcagggttacaggtgggagtgccctttagtcccttcccagtggccaccttcggattcatgtgggacttgtggatccctgcttggtcccactccccgtgagcctctgacacagagtcctcagacctccaccctctccctcccatgtagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggttctgggtgcaagggcaagcaggaggggggccaggaaaggacagttactggaagatggacagcccaggaggctacagagggaaagaaagggggcccctgatgaggatggggagcatggccttgggctcaaacagcagaagggtgagtgtcacctgagcggccacctctcctctccaaggctacatgcggatccagcagtttcggaaggcggatggttcctatgcggcttggttgtcacgggacagcagcacctggtgagcttgggagagtggttccagggttctgagggggtcagggctggggcaggggtgggacagagctggtatgatgggagggtggataaccaggcacctgggggcgtgggcataatgagaagcaagtccttatccccaaccctcctttcctgccctccaggctcacagcctttgtgttgaaggtcctgagtttggcccaggagcaggtaggaggctcgcctgagaaactgcaggagacatctaactggcttctgtcccagcagcaggctgacggctcgttccaggacccctgtccagtgttagacaggagcatgcaggtgcgggcatgctggggctggcccgagaagcgcctgtcggaggactctctttgccccttccccctcctgtttgacatcttttctccccttactaggggggtttggtgggcaatgatgagactgtggcactcacagcctttgtgaccatcgcccttcatcatgggctggccgtcttccaggatgagggtgcagagccattgaagcagagagtggtaagttcagtggcgtttctgccctctgctggcccccagctctctccctttttcctcaggaacccaggggtccaggcccaagaccctcctcccgttttcttccaggaagcctccatctcaaaggcaaactcatttttgggggagaaagcaagtgctgggctcctgggtgcccacgcagctgccatcacggcctatgccctgacactgaccaaggcgcctgtggacctgctcggtgttgcccacaacaacctcatggcaatggcccaggagactggaggtgaggggtgaggcgctcctggcagtgagcctgaggcccaggggaccttaggatccctgagtgtgcccagagggagaggctggatgaagactcagaggaggaatgaagttataagcaggggtgggttgggggagactcaggagagcccagcagggggtggctaagggccaggggaccaggctcttctccctgccttcctgtttactcgtggtctcccttcactttcagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggcttcggcctggctcacccgtcagggcagcttccaagggggattccgcagtacccaagtaggggccgtccccgggctctggcgggggtgggtagtcctcagaccaagggcttgcttgagtcctggctcaacctccctaggacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcaggtgaaccactccctggtgaaccactccctcgcctgggtagccaggacacctgggcctcgtggccaggccagaagccgtccccaccctcccacccgtggaatccccgcagcacttcttcctggggtcttcgggggaagactgacttcctggctgtgtgacctggagctctgagcttcagttttctcacttgtagagtaacatacacagagttcaccctacagggtcgttagaaggctgaagtgagataattcatgtgctggtataaactttgtggaaatgtgaggtggggagaggaggtggggctgttttgaggaaggagataagttattggagccgcaaaaacaggtttgcttgtgcccttctaacatcgccttcccttttctgttgctgaagttttccttgggcagcaagatcaatgtgaaggtgggaggaaacagcaaaggaaccctgaaggtgagggccagggaaggggtggggccaggcactggtggaggagagggtgtggagtgagaggcctgtgggcagaggcacatggtccggggaaggaggcagacacctcagggttggtgtcccgtgcttccgtcctgggtgtttttccccctgcttgctttcgcttgctctccccatctctgggtacctgttgtttcctttacccgcctcagtgctggtggctccgaatcccactcctcagcccaggcctcttccctgaaccatgggccccactcgtcccactcccacagcacctcagacgaggcatgtcccaaagcccttcttcattctgtgtctcttgtctggctggtgggagcccctcccagccaggagcccagccactactctagaggccgtgttagtggcccctctcccaagcctgtccttatgtccctagtgactcctcctctgctcccctgctgcctgtggcccttggtgctgcatcctagattctgtgctgagacggccttctccctacctggaacttctctctacctcctgtctcccctgtctgatccactgtccacacggcagtgacactgaccttccaaaagccccagccagatcagccttggggaaaagtcactccccgctgcccacggctcagatggctgggcctctgcccacccctccggccagacagctctccttgtctacacagatccccttgcctttcctgtccttccctgcttcttggcccacaggacaagctctttcttctccttcaagccttggccagaagcctttcctgagcttttcagtccagcctcttcccagcacagtctggagtgttggcctctgggggcaggcccctgcttctttacctctctgtctcgcctgacgcctgtggcgaatgtggtgccactcgtgtgtgtggactgtgcagtgacggggaggaaaaggggctgaaggcctcaaatcctgtagcccagggagatgcccttaggtatggcaccagagaggtctgtggcctcacatgtcccacgtcctctccctgccccttgctgagccaggtccttcgtacctacaatgtcctggacatgaagaacacgacctgccaggacctacagatagaagtgacagtcaaaggccacgtcgagtacacgagtgagtgtgggggttgggaggccttggggccaggcaggggctggcgcagggagccgggtggccatcccagccctcctcacaatgcttccctgtgcagtggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggtgggcgccgggagctgccctgggccaggggagggagggcaggacccaggctggggctgggcttctggagcccgcgcaggcagaacctggacgacagctcacacgtctccacaggcggaacggcaaggtggggctgtctggcatggccatcgcggacgtcaccctcctgagtggattccacgccctgcgtgctgacctggagaaggtgtggtcagccacccagggcaaccccctctgtcccaggtactgagccctgtcatgtgcagggcctgtgaccaactccccttttccacagctgacctccctctctgaccgttacgtgagtcactttgagaccgaggggccccacgtcctgctgtattttgactcggtgagtggggagagatgaggcaggaagggactcgatggcaccgggtttactgagtatgcgttaggaggtttctcaggagacagctgtgtcagcggctggtgctcttgagaacttgtgatgtcatcagagagaaggacaagaatgtgagcccgtgagacacagcagagtaaggggcagacctgcaggcggcagggaccgatgccagtcagcagggaccctcagggtttgagagggagtctttcctaatgctggttttattcagcttgaggggctgcctttgtttttttgttgaacttcctatcttttttttaatattaaagcgtattttcctttacaaagtgatggtggccatagatgatagttgtatttgtcttttcacgaccttatttggctaaaatagttatcaaccctcttacggctctcaaaacatttttatttatttatttagtaaagacagggtctcgctctgttgcccaggctggtcttgaactcccggcctcaagcgatcctctggcctaggcctttcaaagtaccggatttacaggccagagccaccatgcccggccttcaaaaaaagttttggaacatttactgtaacctctgggagaaaatgtgagaaaggtgtggtggctgtcattagccagctgtttgtaggtcagggagacccctacccagtgtgtgcagaggggccagcccccatcagctggggaagcctggctgacacatctgggttgaacacaatagaaaacacagagccaacaagattcccggatagggagctgacggtgcagcagcctagctcaggagggacactggcacggcaccgtgtggactgggcccgcgtgggcacgaggaggggtcaggcctgggacctgagtcggggggtcaggcaggatgacagaacctgcagttaggttgtggcaaataaaggaggacccagttgtatccatgacaaagatgaggccgcgaggagggcgagtgggtttgggggcaggcagagtgccttggagaacttacaggtcctgccacaatcctaatgcaaggatggagctgcaagttcagtttgggaatcatcagcctggattggtttggtggaagccagggagtggttgagacccccacaggggagctctgaggaaggaagttccgaaggagggaacgtaagaaatgaccaggtcagaaccaagggtggtccagaagctaacccttagcttagggacagtttcacagagaacacgtccatgatgcaagactctgctgagggcctggagcagtgaagactggggcaaggtcaccctctgggaagtgaagtcaccagagaccttgcggagcagctttgagagttctctgagtaggaaggtaacagaatgtgaaggacactggagagaaggccaataggaagcaaacaaaaacaggccaaggaaacccagtacagggggctgcagggcccagggagtgggtccctcatctctcctccccacgcttggccaggtccccacctcccgggagtgcgtgggctttgaggctgtgcaggaagtgccggtggggctggtgcagccggccagcgcaaccctgtacgactactacaaccccggtgagcactgcaggacaccctgaaattcaggagaactttggcataggtgccctcctatgggacaatggacaccggggtagtgagggggcagagagccctggggctccctgggactgaggaggcagaatggaggggcctgtgccctaactcctctctgttctccagagcgcagatgttctgtgttttacggggcaccaagtaagagcagactcttggccaccttgtgttctgctgaagtctgccagtgtgctgagggtgagactgagggcctggggcggggcagtggaggcgggatggccggggccccccccacactgtctgatgggttccccaacttcagggaagtgccctcgccagcgtcgcgccctggagcggggtctgcaggacgaggatggctacaggatgaagtttgcctgctactacccccgtgtggagtacggtcagtcttcccaccgaggccctggcctgaccctccctcggggaccggccgttttggtctctctgggtgtagcctgctcctcttacaggtcatgcacgcagcctgtttgctctgacaccaacttcctaccctctcagcctcaaagtaactcacctttcccccttctcctcaccccctcttaggcttccaggttaaggttctccgagaagacagcagagctgctttccgcctctttgagaccaagatcacccaagtcctgcacttcagtatgaagcaaaccggagaggcgggcagggctggggggagacagggaggctgaggtgtggccgaggacctgaccatctggaagtgtgaaaatccccttgggctgtcagaagccttgggcttggccataaatagggaggcagtggcacctctccatgggggtggcgaaggtggaatgagaggatctacacagagtccccagcctgggctcaccctgcaccttctcttcccctctgaccacttttgcgcacgtcatccccgcagccaaggatgtcaaggccgctgctaatcagatgcgcaacttcctggttcgagcctcctgccgccttcgcttggaacctgggaaagaatatttgatcatgggtctggatggggccacctatgacctcgagggacagtgagtcatctggtcccctcagtctcttgtcctccccatgcctcgccacctaggccttgcccctcagaagccagatgcctgtgctctccgtttccacctgccatcctcccgagccctgctgactgcccctttgccccctgcagcccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggcagtgaagtgtcagtgtgtgttgctagggctgagagcagtgcccctgcccgatgcagttctgggcaggccaggttgacataaccttagactctctgagccctgatgacccttgggctgttcagctctgctagaacctcccagatgacccgctaggagtctagtgcttcacaggaccaccccgagcagaactgggacccaagagcctgcaccccaaggaccagagtccatgccaagaccacccttcagcttccaaggccctccactgcccggctgtcgccagtcaccacggcctcagacagggcttgtgctcagctgacacctgtgacacagctcttctgcctcatgagctgttgtccagctacacctccccgactctgtcctcgtgctgctggcggttctgaggtctgcagattttagctgagttccgggctgttgaaagcctgctgacgcttggttctgttatcagtggaatgaggtgactttcccggagttgtgcaatcctcaggtccggcagtgtcttcttccagttactggtttcaaacaagccaaaagtctgactttggtgtgtttgtgaatcctctgaggaagccgctgttctcctggggtctccccttcccaccggacctgcctaactttcccccatttagtggcacacctggggtcttcagagatgactccgcgtctgtccaaagaagtttggtgagatcagtttccgtagaggtcatgacagttcagcagcctgccatccagtcattcgacagaaattcgggaatctttcacttcatgccatgccctgtgccaggtgccagagatacagctgctcactccagggctcatcgctggggagacagataagaggacgggcagtccccaccctctgtgaaagatgtgatgtcagggagcagtgtggtcctgtggggcatctaaccaagtcaggggcattgccaggcagggacagggaaggcttcctggagcaggtggcctccaagtggggctctgaagactgagaaggagccaggaaaagagcaggggtagatgagggcatctggggcagaaggagaatatacaaaggcccagaggccgggggcaggacagggtacctttggggacattgcatgtaattgaccacattcggagtttggatttggaagtggtggaagagatggagatggtgagacaagtagtaagcacgtcagccttccaggtgcgctcctttccgatgagcactgtcttatcccacgtaactttgagaagtttgggcctttcccactgtggcagaggtttcctgaggctcttgcatacatggccctatggttgctcatcagatctttctcccagtagctgctcagcatggtggtggcataagcccattttccggagccagggattcagttgcagcaagacctggcccggtctgggaggtcaaccatgaagaaggcagtagctgtcattgcccaaccccagaaatcccaatcctgttttctccctctcagtcctgatcatggattcagcagcagcgaactcgccaatgtagtgggtggcacagccagggtcttgactctggctctgcagtagcacagtctggaaaagctctgaggggagagagacccccactggtccgagggtctggcacagagccagaaatgggggggaaggtatggggctgggtcgcctctgacctctcaggtaccatccaggaggccctggcctctcactgaacccggccactcctctttggcatggcctcttcccaaatccccaaactgcctccttactcacaaaagtggtctctgagtgtcagtccagtgggacccccaccccttatggcttcagttccccaaatagggctggacccttgatcctgatccagctgtggctatccagccccttcctggggactttggactttgagggggggcatgcccagttgtgctgggaatccatactttccctggctggagtagaacctgtggactgtagtcctgagggcagtcatgttc

SEQ ID NO: 3 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 4 - DNA, 27624 bp, Homo sapiens
atggtgctggtcctggaggcaccggctccgttctgcatctcctccccgcagtccctggggaaggggatccgcagcccacctgggagaggagagcaggggccagtccttttccaagccttaggccctggctgcccacccagcccccggccccgggcccgtgcgtccaggtacccgtggtgaaagaggtggacacgggcggcaggaggctctggccccacatggcctggagccgtgcattgtaggaggtggagggaaagaggccaaggagctggtgagatgtgatccctcctgggagcaggatctcctgtgggacagacaagggggggtcaggggagagggaggtggagaccctccgggagggccagaggcagcacctcctggaatcacccagggaggggagttgggtcagtggggccggggcacctggttctgtccaccaggggtgtggaagctgagcaggtagcctgcgggccggactgggggctcagtccaagtgagcagggcggtgcggggggtcacttccttggcctccaagtcccgaggggcctctagccctaggagggaaagcaggaagaggagatggggatgaggcccaacctggctccctctacctcctctccctgtcccacacaccccacagaccctacctgtggtgaaggtgatgctggctggggaagtgaggttggggccccgcaggccacgcactgtggcggtgtagttggtgtggaggacaaggtcatgcagggggtagtccaccgcgctgcctggggtctccgcctgcagaggcggggctgggagtgtagagaggggcatcaaggcctgccccctccatcctcggccagagtccagcctcccccctgcaatccccaccctgaacaagtcccctccagaggcctcaggcctgctcacccccaggggctgtgacctggacgtcataggtgtccacaggattctgggggggcttccagtgcagcacggcgaatccctcggtcaagttcagtgcacgcaactgtgtgggaccgtcaggaactgggggaaggggaggggctcagaagggtccccgcggctctctctactccgtgcctccccagactccactggcctcccgtccgcaatcggagcctccaccacctccctttcaccctcctcgttctctctcaactcccacccatgccgttttcttgactcccacctggagtttctgggtccgggcccggccgtccacctgcacactctgaggctcccctgaaaacgttggggatcgagggttacccagggaaccccagggcggctggagggtgggcagagtgcaggggggagaggaaatgcgaggcgatgagcacatggcaaaggcaccacctccgtccgccagctggtaggagactttgaagctgtccgcccgggatggtgggggcatccagttgaccttggctgaggtctccctgatttcactgaattggaggtcacgggggctctccagaactgcagaggggtcaaggaacaatgacgcaggcaggggcagggaggctcctccctgcgagtccccccctcgcctctgctccagcacaggctcaccaccccttttcctctagtccccaggaatggaagtcgctctgcagattcctccaggcccaccaccaactcgcccacccccaccgctggctgaggcactaggtcccccccgtgaagtacaaagacccccactttggggcagagtgtgtgtgggtccttacctgggctgagggtgcgggcggttccctggatgctgtcggccttgtggggtcctcgcagcccatacagtgtcaggctgtacagagtcccggaacgcaggtcccggagcacggccgagtgccgcgtccccggcaccatcagctcgcgctgcagcagtggacgcggatgcggctccagagtgcttggtgatggaaccccaaagcggagcaggaaggagtcgaaggcccccggtggggcctcccagttgagcctcagtgaactggtggtcacgtcagtcacagacagctgggacaggcggggccttgactcctctgaggtctgaccagcaggagccagccctgcacggagtgggtgggggagaagggattggagacagaagcacaccagcttggtgacccagagcacgtcccttccacccccctccctgcccccgtttctctatctgtaaccagggacttgcagccacaggggggtcctgtggggcagagctaaaggccactcgcatccagcccatccatcctctctccctggtacccgcctcacgctctttccctgcgaccaccccttctgagcccccgtttctcccttctgagtcctaggctagaggccggagacgcctggtggtacctgtggtgccctcagctgagaggggccccaggcgcttcccttcatggaggccatagaggaggaacctgtagcgggtgctgggctccaggcctgagatgaggatcttgctctggtcgccgtccacgagcaaggcctggggctgcccattcgtgtcctcatactggaccacgaaggaatcaaaggggccctgggccacgctccacgagaggcgcatggagtctggggttgtgtcggtcacggtcagcactcctaggcggggctcttcaggaggctcaggggcctctggggctaactctggggctggtgtgtcctcttctggggctgcgtgggagaagcccaggggagaatctgagtgaggggcgccatggggtgctccatttttatcttccaggcttggcccaaggctgaggtgggaagtttataggtccaggcccagtcagacaatgaagtcgctgtggcctcgtgactcctgcgagctcccgcgctgtctgagtcaggtgctcgcttcccccttccacaccccggtgtcctgccgagcccacctcgagatatcacaggctctggccccacccatgccgggatacattcactgagcttgaggagtgtggtgctcccttctgagagaagctgagggtggaactggctggttgaggtgactggcaaatcccaccagccgtgccgtggtcaggcctgtctgaggtgggcatcagcgagctctggaagaggagcctgtaccacaaatgcagccactgctgttggtttctgtgtccccgctcattttgttttccagtgatgttcctcttaagaaaatgctcctgactcatccacggcagggaggtttgccactatctggacaaggccacccttcggggaggcgacagcagccccagcgagtaatgaggagcagcggcagtgacggggcagagtcggggctgggagattagagagcccctcccagggcctttccctcccgcctggcctggctcctgctctggactccttgatggatgttgaagcccacagggctgcagactcctcctccttcctgggcacaggccaggtcaccccactccggcctgcccactcctgcagtcatctttgtcttcagaccaaatgcacaagtactttgttaaaggtatcccatctgcagctcaagcctgcagcccctcaccttttggtggctcctcaggcctctaggccttattcacctttcccctttcctgtgccacttctcctctagggcgccaggctgtccttggcatggtccggaaggcaaagtaccgggagctgctcctatcagagctcctgggccggcgggtgcctgtcgtggtgcggcttggcctcacctaccatgtgcacgacctcattggggcccagctagtggactggtgagtctttccctggcctctggcagattatggagcaatgacccaaagtgggatttcctcccagctcatgcttagtttcctagtgaaggccagtggctctcattcttctctggaacccgggagcaccccttcccaagttctaagttctcctcacagcttgagcctaggcgtctggctccagccttgtctttctcctgcacagcatctctaccacttcaggaaccctcctccgcctgccagagacatgaagattctgctcatcattgctcagctcctcagagtgggccgggaggggactagaagagctgcatgatggtggctgagacagggtcaccttgggaaggcttgggagccaggatgagtgtcgggctctcgtgtgtgcaaaaggtcagatgtgactgctgctgtttgcctggtttctgacccagtggtggggtttgagcaatgcttctctgcccttccatggaaagtggaaccagaaatggtgccaaggctgtggctgttccctttcgtgtaaaatggtgctgttattactctgtcttgaaataggaaggtgggatttctggggaggctggtgaaggagggcagggttcttttctctacgtgtcatgttaaaattgccaaataaagtacctctgcctgtgatattttctggatgtcctttatttactgtgacgtgtgtttgggtgccttgtttaggggtagaggtgaagtctgagctttgcctcattcagagaggaaaggggtcaggggttcactctgacgttcaggccattctccctgtggagtggtgagggtgtacctaatctcctaaaccacggaatttctgttagggcctaaaaaagcaaaagcctagtatagttcaatttgtgttggaatgaaagtaagagacaagtgtcttagaagcctgtcattgttttgtgagggcctttaaatatcctgtactcgtgggccatgttgggcccttgtacgcccaggtatacatgagcttgtgtgcacctataccctgatacagatatacctggtagggggaggtgctcaggcactggaatgagaggagttaacggggaaggacagggttatttctgggccaagattcagagtttcccatggacacccaggtgtccggggtgcccccacaactctgggcctgaggccagttgcacttcttggctgtcacgtggtttcccagcttagctgggctgggggaggagcaaggtccagagtcaactctgccccgaggcctagcttggccagaaggtagcagacagacagacggatctaacctctcttggatcctccagccatgaggctgctctgggggctgatctgggcatccagcttcttcaccttatctctgcagaagcccaggtcctggaggcgggatgctgggtgcttggattggggcagggctggcatcgggacccgattcaggagtgagggagagcaggggtggaggtgtcagagcgaagtctgactgctgatcctgtctgttctccccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtaaccagaccccatgccctcctgctgcttgtgggggcctcctgccctgttcccatctgtcttgtaagtgtcatcatcttcccactggcctcctcccctcctgtcttcccaccctggcattctccttccacgtttctcccttggtctctgtcctttttggtcagctgtctcttgctctgtgacccgctccctctccctctccctctcctgacaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcggggtgagtctcagccccagggcctcaacctttaaccccctccgagccctctcaggatgagtttggtgccccctaagtgagataacctgaaagaaagtgccacacagaaggggtgcttaggaaacatttgtcccctgctccctctgtggagtttgacccaccctccccttgcacatggacccctgctcacctctctcctcctccactcccagttcggtaccgggtctttgctctggatcagaagatgcgcccgagcactgacaccatcacagtcatggtggaggtgagtccccgacctctggccttcctgatcctggccactgatgtgacctcctgcctgtgagcacttctccccttgcagaactctcacggcctccgcgtgcggaagaaggaggtgtacatgccctcgtccatcttccaggatgactttgtgatcccagacatctcagagtgagcgctcccaatgtgggggctgcccccaagctacaccaccccaattcctgttaggctctccacctcccacacagaggcacgtccccagatgccctgaccctcagcctcctgagcctctggttaacccccacagtcctcttcccagggaagcaggctgctggctctccgtgccccactgtacagatgggctgagccccttccttgtccattctcaggccagggacctggaagatctcagcccgattctcagatggcctggaatccaacagcagcacccagtttgaggtgaagaaatatggtgagagctggaaactggagggacaggcagctgctttcctgaaggaaataagggtggaaggagaggtactgggagcagctcagggcagggagatatgggtgccacagccctgagcagaggggagtctttgagctggagtctgacctgcctatcccttcaccctgggtcagtccttcccaactttgaggtgaagatcacccctggaaagccctacatcctgacggtgccaggccatcttgatgaaatgcagttagacatccaggccaggtaatacctccctccccacctctgcccaccagcaccgggtcctgctccctactcagtatgaatgggctcctgcttccctgccctcgggccattattccccccagcccttggcccaccctcttctctctgccacgacaggtacatctatgggaagccagtgcagggggtggcatatgtgcgctttgggctcctagatgaggatggtaagaagactttctttcgggggctggagagtcagaccaaggtaggaaggagaataggggctggggaggggaaggggcaagggaggtgaggtgggagactcagtctcaccctatgtcctgtttctttctatgccccagctggtgaatggacagagccacatttccctctcaaaggcagagttccaggacgccctggagaagctgaatatgggcattactgacctccaggggctgcgcctctacgttgctgcagccatcattgagtctccaggtgggtgactttcccttattgtaaccccagacccttgcctctgacctctgagctaaccctctgtcctccggcaccaacaccaccccacttctcacatctcatctcagactcaaaaccaggaaacacccaggagacctggtttctctccaactctgtctctgtgactcggcccttttccctggctgagtttatttatttctttgctcgttctgctcattccttcactcctccagtggacatgtgttgttcaatgccccgtgctaggcctcagcatgcacagacatgttggggaccagcctcaacgccacccgtagggttcctgaagtccattggtgacacaggaatgagaagagacaggttaagagttcataaagagtgggggccagggggccaattgcaaaatggaggctgcaaaaggctcagagctctggtctccacactattttttgagtacagtcactcagatctaagaagcagatgttcagggagaaacagtgaaagggaggcagtgggtcataggcgtaatctatagcaatagagttttaaatgaatctcctttgtgctcaaacagcatgtctttaaattatcggagagtagctggtggaagtgggcttagctagaagactgcatgtctgtccaatgcttcaaaggagggtctttctccttgaacagagtgtttacagataagacagggggtctcactctgagcatgggaacatgatggcaattaggaggcttttcttctcagaggcctcttgtggctttccacaacttattgtctcatatttttatggacagtttatacaggcaccccacaagtccttttcccaacatgcccccctccctttttttttttttaaccgctattgctattatggcttatttgtggtgtttggtctgttttcagaagtgtcttttgcatctgtagactaaaagtaaacagcataaacagatacacattaaagtaaaatttgtaatagttgatcctttaatggtcttaatctgtttaagaggatttatgtttgaaagtccgtcagtagctccaatgagaatgtcagtctcaggcaggagggttaaatgagcctgagatgctttaaaaacctgtttttttaaaatttggttatatttaatgttaaatttttatttttttcttttagatgatgtctaactttttaaaaatgatgtttagtagtattatacgaatggggagttatgtagaaattggaagtatttcaattacattgtacttctaattgatgttttaagtttattgtacgatcttccatttaaataacagtctgtctaagatcatttgtttgatttgtcaattgttggtctatttgggtctgagaattccacaattttgaggaattttttgttaactatttatatattttgtagtttgaacagaggagtgtaaagcaattccagcagccgcagcagtagctgtgactgcaataaggcccataagactgttataagggtaaaaataaatctctttgttttggtaaacacttttttttaaaacatttttgtgacaatatgaatggaaggagaggctttctaaggtctattgagggaaaccagtatccaaactcctttcttagtttttatcagtaacacagatgtttttacaccgaacgtggaattaatacaggtgaaaaggtgacagttttgacaagtaatagtttgagaattaggtcgaatgtcaatatttttgaccattaacataaaaggagggttgacacaactctgaatgggcactgttttgttggaagaaaactgatacgcaaattgaagtttttaaccttttttttttaaagataatatatttttttctaaacttaaatatgagattgggccattattaactttcataatttggagtgtttagggcctattattggattaattattttgggatgtgggccagctgtactaaaattggtccaaattatgggaaaatgagcacgtttttcagtgtaagtagtgttacctttttgatagtatagtttctgttttagttttgtcttgtatttattattttgatgggtacaattaactgtaaaggtcccctcaggggaccaattaatgacaatttcataggaattattttgtagtaccatagtgtgatcagagatgtaattttttttaattaatatttttaaattatttgaccattgttaaggttgttggcacctcttttttgggggcttaaactgttaattgaattgaactctgtgaatgatccgggctccatccagaaaataaatgataggatactggtctttgattatgacctggaattttaactagtcaatgttgtcggtagccttttaggcaaccgatagttggccttatgtaaagaggggggaactgataacctatggacacatttattaacttttttttttttcctttgggtgagagggcccatgagtatttgtaggcttagggatccaaacgctattattaacataaacttcaactgggggttttaaccatgtgacaggcctaattaaaggcaggaatgggacacatgcccaataggtataattttgggctgttgtagccacaggtttgttaggcgaggaggtcactgtttttattttggctttgtattctaggattagtaaataacagaagacaaacatgagtataattagtaacttttttttttagtaaaagagtgacctgtagtgttacttggcatcttagtttactatatgttattaatgaggaaccccactgggggtatgttaatttattctagctaagcagttatgttattagaagctgagaagggggtgtttgttaaagtaacagggcagaagaaaggcggatttaagatacgagcttaatacagtgtagcaggtataggtagtaggcaaagtgagagaattaaaaatgaataaattatttggcttagacttttgtttttttagtataatgtctgaggcctgtgttgtttgtggaagtcgcattgttgaggctgtagttcctgtagggtcttttttaggctggttcaaatgtttttttattttttaattttttatcctttgatgaggatgtagtctttaggctggtactggaaattttaggagtggcgtctgtgttaagagactttttacaatttttaaagagcaggttagtgttttaagaaaaacttgtgttttattttaatgtttagtttatagaaaactggatgatatctttttaactttagtaaatacgtttacacacggaattttttacaattatcattttaaaacttgtttagatctttaaaacaaaattaaacaaccttttttgtataaattttttataactttttttatgacttttacagacaatttttaacatgtcttaactttttatgttttataatttttttactaaaggtacatttttataactttttaaatttttttacttttttgtatttttttgatttttgtcttagtcttttttttacttttatttttttaaatgtgtaataattagatgagtgttggtaacaatggatgtatgtacatattttagtttttaaaatttagggatgtgtttaacatctgtttgccagaactgactaggttccaattctttacggttaacacctattgaaggagggtatgtgcctgtgagctggtaatctgggcattgtgggataatttgtttagccagcctctgtgtaagttgaaattatttagataagtttctccaattttggtggaataatcgatgtgattgggtggcttggtcaagcagtgatgtcataacctgaaggtctgcttgattattgccgtaagccaatgggccaggcagagagctgtgggctcgaatgtgtgtaataaaagtaggatgtgtaccttggtctagtaattgttgaagttgaagaaaaagaccacacagagtgggctccagagcaaacttaaggctgtaatagtttttaaataaatacacagaataaccttagctctctgaatgttagtaaattcagatcaagtgattggattatgtggtctccaccagactgttgctttttcatgtttaccagacccaccagtaaaaacagctatggctccttccaaaggggcatcacaagtaatttttggaagaacctatgtagttaattttaagaattgaaaagtttttaggataatgattattaatacatccaacaaattttgttaaattaatctgtcatgtaactgagttaataaatgcctgtttaacctgatttttatttattggaactataatttttattgggctcagtgccacaaaatttaataattcatatatgagcctgtccaattagaattgccatctgatttaagtatactgtaagtgcttttatggtattatgtggcaaaaaggaccatttaactaaatcatcattttgaacaataacccccattattgtgtggttagtgtgaagtagggaacacaatgaattataaaggcaagtctgagtcaatcctactgacctgggcttgctgaattttgttttcaattactgataactctttcatggcctcgggtgttagttctctgttactgcgtaagttggtatttcccctcaatattgagaagagattagacatagcataagtaggaattgctaaattgggccaaatccaattaatatcttctaacaatttttgaaaattatttaaggttttgaaagaatctcttctaatttgaaccttttgaggcttaatggctctatcctgtacttgtattttcaaatactgaaaaggagtggttgtttgaattttgtcaggtgctataagtaattcagcatttgtaattgtcttttgcaaagattaataatattgaataagttggtctctactttttgctgcacaaatctggaaactgatctctaacaggctggatagttctgcctacaaaagtttgacaaactgtgggactatttaacataccctggggcaaaactttccaatgatatttggctgcaggttttttgttattaacggcaggaatggtaaaggcaaattttttgaaatctgcctctgctaaaggaattgtaaaaaagcagtcttttaaatctataataacaagcggtcagtctttagggagcacagtgggggatgggagcccaggttgtaaggctcccatcggttgaattacagcgttgacgccatctaccggactttttcttaattacaaatactggggaattccaaggagagaaagtgggtgaaatatatcctttttttagtagtttattttataaagcacccccaacttttccttagggagcggccactgttcaacccagacggggcgccgggtcatccattttaagggaaattgctccttcactgtaataactgtagggtgaacctgaattgccccatctccataatgaactgtgggtcgggcaataatgggcacggtgagccaagtctcgggctccctccccctgcacccactcggctgaggaggaggtggccattctggacatttctctacaggaaccgtgggctgaacaattttttgagtaggtttagggagactggggagattggcataaatcatcttcagactctcctttttgttagtactcggtagaggtggttcagagttctgattatcaaactcctctctctcctcctctgactcagcctcattatctgtctgaaaaggctccagtgctgcatgcaccaatgaccaaagcgaccaaacaggcaaaggaatttcctttccttctctatatgctcttttaaggtcctttccaactccttcttaatgttttaatttcaaagtttcctgttttgggaaccaagggcaaaattgttccatagcatgaaacaaatccataagattttccgtatcaacttttaccccaccatgcatgcttgaagagctgccgtaggaagctcaaatacgtggtgtacttactttcagtttttcccattgtgtccctagctttctctgggcgccccgcttacctgtagaggttaaaacttttatgtccttgggagtcctttgttcgttggtcctctgtttcacatgcttgagcgtttcctcaccagattcttttgggccccacgttgggcgccagaatgttggggaccagcctcaacaccacctgtagggtacctgaagtctggtggtgacaaaggaatgagaagagacaggttaagagttcataaagagtggaggccagggggccaattgcaaaatggaggctgcaaaaggctcagagctctggtctccacactatttattgagtacaataacttagatctaagaagcagatgttcagggcaaaacagtgaaagggtagcagtgcgtcacaggcataatctacagcagaagcgctttaaatgaatctcctttgtgctcaaacagcatatctttaacttatcggagagtagctagtgggagtgggcttaactaggagcctgcacgtctgtccacattccaatgcttcaaaggagggtctttctccttgaatacagtgtttacagataagagagagcaggtctcgctctgagcatggcaattaggaggcttttctcctcagaggcctcttgtggctttccacaacttattgtcccatatttttatggccagtttatacaggcaccccacaagtccttttcccaacacagacaggaatacggcagcctgtgccctgggagctcactgtcttgtgggagggaaccactcaagccactccccacttgtcctcctgtccctctcttcttgggctctgtcccccacctctctctgtcctttgtcttgcaggtggggagatggaggaggcagagctcacatcctggtattttgtgtcatctcccttctccttggatcttagcaagaccaagcgacaccttgtgcctggggcccccttcctgctgcaggtttcttccagaggggaaggatgagtagggaggatgtggtagttaggagggctcagggtctgaccactctcttttgcctgccctcctttacctgcctaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtaggactcctcggacccctgggagatggtgggggaaggggaggagggtgagctggggtcccaaggatccatggcctgacttggggggaaggtggggtacttggctctgagctactaccctattcgcacctgaccccctctccaggtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatggtgtgcatgagctggggagtcacggagggctggggtgcagggaagagccctctgggtggggctgggggggttcaaggctgaggctgtcccatgaagaggcaaccactcttgtccctcccattcttggcccagatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaaggtgaccggggtcaggagagatggcacttgtgccgagggggttgaggacagggtgattgccaacagggcatggatttagcttgggggcagtgaggataccgggactgaaggaagctctcccactctgaccgcccccacctgccgcccctgccagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtttgtccagaccctctccacagctctctcacccctccatggctcatccccctgcttccctgagccttgggcgcagcccctggatcccactgaggctccccacagtctcttccccacttggccctgtggtctccatctcctggctctgtatcctttcctatccccccatgtgctgccctctcacctgtgccgagtgctcagtcctgcccctcagccacacttggctcctagcattcctgcctttcttgcaggtctttgaagctatgaacagctatgacctcggctgtggtcctgggggtggggacagtgcccttcaggtgttccaggcagcgggcctggccttttctgatggagaccagtggaccttatccagaaagagtgagaacagagaaggaaggggagtgggtggcgggaagataaggaaggaggaagggcctgaggggaccagctggaagagtccgggcaggaagggctgggcaggggaaggggaggaggggaggaggccgagtgcctgacggctggactgcagcctttctctctaccaggactaagctgtcccaaggagaagacaacccggaaaaagagaaacgtgaacttccaaaaggcgattaatgagaaatgtgagttgcgggtgcctaggcagtagcttgggctctccacctgggatccgggttgggggtctgcctctctgcccctcggctccttgctgaacccacgtgtggtatttggggccagagatccgaattccgggattacgagtggaaggtgggcagctctctccagcagcctctcttatgttgctggtctcaaggggtcggggcgggggctgaggtgtatgtcctttttgtcctctcatgctcacccccacctggccctgcagtgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgaggtgaggggctgggtggggctagggcacaggtggcggcgcttggaaaggcagaacggtcccctcctcactcccgtccaccgtggtcccccagccctggagatcctgcaggaggaggacctgattgatgaggatgacattcccgtgcgcagcttcttcccagagaactggctctggagagtggaaacagtggaccgctttcaaatgtgagagtgtgtgccggcccggccttttctctgtgctgtgtctcggggccagccggggtagacgggccttctctgcctttccctacacagattgacactgtggctccccgactctctgaccacgtgggagatccatggcctgagcctgtccaaaaccaaaggtgatgtcaccctgtctgggcctcaggtgaccctgcttccatttccctgtaccccagctccctgttccctttgctcttagtgtaggaagagggtccagtgatctggggaggtctgtgccagcgtgcagctggcgtgggccagagggcagaggcggactgagacagagctgggtcacccccacccctccctcctgtggccctgaagctttgatggcccctctgatctctgcccctgtgcccacgcttcctttccctcaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgaggccccatgggagcctgagcatacaggagttgggggagccagggcccagtgaggggtggggaggctaaccgggccaggactctggccatcctcgttttcctgccctcaggtgagcgtccacgtgtccccagtggaggggctgtgcctggctgggggcggagggctggcccagcaggtgctggtgcctgcgggctctgcccggcctgttgccttctctgtggtgcccacggcagccaccgctgtgtctctgaaggtggtggctcgagggtccttcgaattccctgtgggagatgcggtgtccaaggttctgcagattgaggtgaatggagcacccctgaatataagtccccgggcccccagctttgtcctccaccctcagcactctctctgctggccaggccaggggcccaacacccaaaccaatgccttggtctgttcccatcttctacaattctgatccaactctgtccctggagttgaaactcaaagttctgggggagtctgcgctagcagggcaggctgtagtcctgtgtgacctcacaaccatgttttccctgagacagaaggaaggggccatccatagagaggagctggtctatgaactcaaccccttgggtgagtgaccctctacctccagccattggtttcctaagtgggtacaggtggtgggggatgtggacagcaggacaggctgccaacttcccccatttccccagaccaccgaggccggaccttggaaatacctggcaactctgatcccaatatgatccctgatggggactttaacagctacgtcagggttacaggtgggagtgccctttagtcccttcccagtggccaccttcggattcatgtgggacttgtggatccctgcttggtcccactccccgtgagcctctgacacagagtcctcagacctccaccctctccctcccatgtagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggttctgggtgcaagggcaagcaggaggggggccaggaaaggacagttactggaagatggacagcccaggaggctacagagggaaagaaagggggcccctgatgaggatggggagcatggccttgggctcaaacagcagaagggtgagtgtcacctgagcggccacctctcctctccaaggctacatgcggatccagcagtttcggaaggcggatggttcctatgcggcttggttgtcacggggcagcagcacctggtgagcttgggagagtggttccagggttctgagggggtcagggctggggcaggggtgggacagagctggtatgatgggagggtggataaccaggcacctgggggcgtgggcataatgagaagcaagtccttatccccaaccctcctttcctgccctccaggctcacagcctttgtgttgaaggtcctgagtttggcccaggagcaggtaggaggctcgcctgagaaactgcaggagacatctaactggcttctgtcccagcagcaggctgacggctcgttccaggacctctctccagtgatacataggagcatgcaggtgcgggcatgctggggctggcccgagaagcgcctgtcggaggactctctttgccccttccccctcctgtttgacatcttttctccccttactaggggggtttggtgggcaatgatgagactgtggcactcacagcctttgtgaccatcgcccttcatcatgggctggccgtcttccaggatgagggtgcagagccattgaagcagagagtggtaagttcagtggcgtttctgccctctgctggcccccagctctctccctttttcctcaggaacccaggggtccaggcccaagaccctcctcccgttttcttccaggaagcctccatctcaaaggcaagctcatttttgggggagaaagcaagtgctgggctcctgggtgcccacgcagctgccatcacggcctatgccctgacactgaccaaggcccctgcggacctgcggggtgttgcccacaacaacctcatggcaatggcccaggagactggaggtgaggggtgaggggctctggcagtgagcctgaggcccaggggaccttaggatccctgagtgtgcccagagggagaggctggatgaagactcagaggaggaatgaagttataagcaggggtgggttgggggagactcaggagagcccagcagggggtggctaagggccaggggaccaggctcttctccctgccttcctgtttactcgtggtctcccttcactttcagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggctgcggcctggctcacccgtcagggcagcttccaagggggattccgcagtacccaagtaggggccgtccccgggctctggcgggggtgggtagtcctcagaccaagggcttgcttgagtcctggctcaacctccctaggacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcaggtgaaccactccctggtgaaccactccctcgcctgggtagccaggacacctgggcctcgtggccaggccagaagccgtccccaccctcccacccgtggaatccccgcagcacttcttcctggggtcttcgggggaagactgacttcctggctgcgtgacctggagctctgagcttcagttttctcacttgtagagtaacatacacagagttcaccctacagggtcgttagaaggctgaagtgagataattcatgtgctggtataaactttgtggaaatgtgaggtggggagagggggtggggctgttttgaggaaggagataagttattggagccgcaaaaacaggtttgcttgtgcccttctaacatcgccttcccttttctgttgctgaagttttccttgggcagcaagatcaatgtgaaggtgggaggaaacagcaaaggaaccctgaaggtgagggccagggaaggggtggggccaggcactggtggaggagagggtgtggagtgagaggcctgtgggcagaggcacatggtccggggaaggaggcagacacctcagggttggtgtcccgtgcttccgtcctgggtgtttttccccctgcttgctttcgcttgctctccccatctctgggtacctgttgtttcctttacccgcctcagtgctggtggctccgaatcccactcctcagcccaggcctcttccctgaaccatgggccccactcgtcccactcccacagcacctcagacgaggcatgtcccaaagcccttcttcattctgtgtctcttgtctggctggtgggagcccctcccagccaggagcccagccactactctagaggccgtgttagtggcccctctcccaagcctgtccttatgtccctagtgactcctcctctgctcccctgctgcctgtggcccttggtgctgcatcctagattctgtgctgagacggccttctccctacctggaacttctctctacctcctgtctcccctgtctgatccactgtccacacggcagtgacactgaccttccaaaagccccagccagatcagccttggggaaaagtcactccccgctgcccacggctcagatggctgggcctctgcccacccctccggccagacagctctccttgtctacacagatccccttgcctttcctgtccttccctgcttcttggcccacaggacaagctctttcttctccttcaagccttggccagaagcctttcctgagcttttcagtccagcctcttcccagcacagtctggagtgttggcctctgggggcaggcccctgcttctttacctctctgtctcgcctgacgcctgtggcgaatgtggtgccactcgtgtgtgtggactgtgcagtgacggggaggaaaaggggctgaaggcctcaaatcctgtagcccagggagatgcccttaggtatggcaccagagaggtctgtggcctcacatgtcccacgtcctctccctgccccttgctgagccaggtccttcgtacctacaatgtcctggacatgaagaacacgacctgccaggacctacagatagaagtgacagtcaaaggccacgtcgagtacacgagtgagtgtgggggttgggaggccttggggccaggcaggggctggcgcagggagccgggtggccatcccagccctcctcacaatgcttccctgtgcagtggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggtgggcgccgggagctgccctgggccaggggagggagggcaggacccaggctggggctgggcttctggagcccgcgcaggcagaacctggacgacagctcacacgtctccacaggcggaacggcaaggtggggctgtctggcatggccatcgcggacgtcaccctcctgagtggattccacgccctgcgtgctgacctggagaaggtgtggtcagccacccagggcaaccccctctgtcccaggtactgagccctgtcatgtgcagggcctgtgaccaactccccttttccacagctgacctccctctctgaccgttacgtgagtcactttgagaccgaggggccccacgtcctgctgtattttgactcggtgagtggggagagatgaggcaggaagggactcgatggcaccgggtttactgagtatgcgttaggaggtttctcaggagacagctgtgtcagcggctggtgctcttgagaacttgtgatgtcatcagagagaaggacaagaatgtgagcccgtgagacacagcagagtaaggggcagacctgcaggcggcagggaccgatgccagtcagcagggaccctcagggtttgagagggagtctttcctaatgctggttttattcagcttgaggggctgcctttgtttttttgttgaacttcctatcttttttttaatattaaagcgtattttcctttacaaagtgatggtggccatagatgatagttgtatttgtcttttcacgaccttatttggctaaaatagttatcaaccctcttacggctctcaaaacatttttatttatttatttagtaaagacagggtctcgctctgttgcccaggctggtcttgaactcccggcctcaagcgatcctctggcctaggcctttcaaagtaccggatttacaggccagagccaccatgcccggccttcaaaaaaagttttggaacatttactgtaacctctgggagaaaatgtgagaaaggtgtggtggctgtcattagccagctgtttgtaggtcagggagacccctacccagtgtgtgcagaggggccagcccccatcagctggggaagcctggctgacacatctgggttgaacacaatagaaaacacagagccaacaagattcccggatagggagctgacggtgcagcagcctagctcaggagggacactggcacggcaccgtgtggactgggcccgcgtgggcacgaggaggggtcaggcctgggacctgagtcggggggtcaggcaggatgacagaacctgcagttaggttgtggcaaataaaggaggacccagttgtatccatgacaaagatgaggccgcgaggagggcgagtgggtttgggggcaggcagagtgccttggagaacttacaggtcctgccacaatcctaatgcaaggatggagctgcaagttcagtttgggaatcatcagcctggattggtttggtggaagccagggagtggttgagacccccacaggggagctctgaggaaggaagttccgaaggagggaacgtaagaaatgaccaggtcagaaccaagggtggtccagaagctaacccttagcttagggacagtttcacagagaacacgtccatgatgcaagactctgctgagggcctggagcagtgaagactggggcaaggtcaccctctgggaagtgaagtcaccagagaccttgcggagcagctttgagagttctctgagtaggaaggtaacagaatgtgaaggacactggagagaaggccaataggaagcaaacaaaaacaggccaaggaaacccagtacagggggctgcagggcccagggagtgggtccctcatctctcctccccacgcttggccaggtccccacctcccgggagtgcgtgggctttgaggctgtgcaggaagtgccggtggggctggtgcagccggccagcgcaaccctgtacgactactacaaccccggtgagcactgcaggacaccctgaaattcaggagaactttggcataggtgccctcctatgggacaatggacaccggggtagtgagggggcagagagccctggggctccctgggactgaggaggcagaatggaggggcctgtgccctaactcctctctgttctccagagcgcagatgttctgtgttttacggggcaccaagtaagagcagactcttggccaccttgtgttctgctgaagtctgccagtgtgctgagggtgagactgagggcctggggcggggcagtggaggcgggatggccggggccccccccacactgtctgatgggttccccaacttcagggaagtgccctcgccagcgtcgcgccctggagcggggtctgcaggacgaggatggctacaggatgaagtttgcctgctactacccccgtgtggagtacggtcagtcttcccaccgaggccctggcctgaccctccctcggggaccggccgttttggtctctctgggtgtagcctgctcctcttacaggtcatgcacgcagcctgtttgctctgacaccaacttcctaccctctcagcctcaaagtaactcacctttcccccttctcctcaccccctcttaggcttccaggttaaggttctccgagaagacagcagagctgctttccgcctctttgagaccaagatcacccaagtcctgcacttcagtatgaagcaaaccggagaggcgggcagggctggggggagacagggaggctgaggtgtggccgaggacctgaccatctggaagtgtgaaaatccccttgggctgtcagaagccttgggcttggccataaatagggaggcagtggcacctctccatgggggtggcgaaggtggaatgagaggatctacacagagtccccagcctgggctcaccctgcaccttctcttcccctctgaccacttttgcgcacgtcatccccgcagccaaggatgtcaaggccgctgctaatcagatgcgcaacttcctggttcgagcctcctgccgccttcgcttggaacctgggaaagaatatttgatcatgggtctggatggggccacctatgacctcgagggacagtgagtcatctggtcccctcagtctcttgtcctccccatgcctcgccacctaggccttgcccctcagaagccagatgcctgtgctctccgtttccacctgccatcctcccgagccctgctgactgcccctttgccccctgcagcccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggcagtgaagtgtcagtgtgtgttgctagggctgagagcagtgcccctgcccgatgcagttctgggcaggccaggttgacataaccttagactctctgagccctgatgacccttgggctgttcagctctgctagaacctcccagatgacccgctaggagtctagtgcttcacaggaccaccccgagcagaactgggacccaagagcctgcaccccaaggaccagagtccatgccaagaccacccttcagcttccaaggccctccactgcccggctgtcgccagtcaccacggcctcagacagggcttgtgctcagctgacacctgtgacacagctcttctgcctcatgagctgttgtccagctacacctccccgactctgtcctcgtgctgctggcggttctgaggtctgcagattttagctgagttccgggctgttgaaagcctgctgacgcttggttctgttatcagtggaatgaggtgactttcccggagttgtgcaatcctcaggtccggcagtgtcttcttccagttactggtttcaaacaagccaaaagtctgactttggtgtgtttgtgaatcctctgaggaagccgctgttctcctggggtctccccttcccaccggacctgcctaactttcccccatttagtggcacacctggggtcttcagagatgactccgcgtctgtccaaagaagtttggtgagatcagtttccgtagaggtcatgacagttcagcagcctgccatccagtcattcgacagaaattcgggaatctttcacttcatgccatgccctgtgccaggtgccagagatacagctgctcactccagggctcatcgctggggagacagataagaggacgggcagtccccaccctctgtgaaagatgtgatgtcagggagcagtgtggtcctgtggggcatctaaccaagtcaggggcattgccaggcagggacagggaaggcttcctggagcaggtggcctccaagtggggctctgaagactgagaaggagccaggaaaagagcaggggtagatgagggcatctggggcagaaggagaatatacaaaggcccagaggccgggggcaggacagggtacctttggggacattgcatgtaattgaccacattcggagtttggatttggaagtggtggaagagatggagatggtgagacaagtagtaagcacgtcagccttccaggtgcgctcctttccgatgagcactgtcttatcccacgtaactttgagaagtttgggcctttcccactgtggcagaggtttcctgaggctcttgcatacatggccctatggttgctcatcagatctttctcccagtagctgctcagcatggtggtggcataagcccattttccggagccagggattcagttgcagcaagacatggcccggtctgggaggtcaaccatgaagaaggcagtagctgtcattgcccaaccccagaaatcccaatcctgttttctccctctcagtcctgatcatggattcagcagcagcgaactcgccaatgtagtgggtggcacagccagggtcttgactctggctctgcagtagcacagtctggaaaagctctgaggggagagagacccccactggtccgagggtctggcacagagccagaaatgggggggaaggtatggggctgggtcgcctctgacctctcaggtaccatccaggaggccctggcctctcactgaacccggccactcctctttggcatggcctcttcccaaatccccaaactgcctccttacccacaaaagtggtctctgagtgtcagtccagtgggacccccaccccttatggcttcagttccccaaatagggctggacccttgatcctgatccagctgtggctatccagccccttcctggggactttggactttgaggggggcatgcccagttgtgctgggaatccatactttccctggctggagtagaacctgtggactgtagtcctgagggcagtcatgttct

SEQ ID NO: 5 - DNA, 7658 bp, Homo sapiens
tgtggggaaaagcaagagagatcaaattgttactgtgtctgtgtagaaagaagtagacataggagactccattttgttatgtgctaagaaaaattcttctgccttgagattctgttaatctatgaccttacccccaaccccgtgctctctgaaacgtgtgctgtgtcaactcagggttgaatggattaagggcggtgcaggatgtgctttgttaaacagatgcttgaaggcagcatgctccttaagagtcatcaccactccctaatctcaagtacccagggacacaaaaactgcggaaggccgcagggacctctgcctaggaaagccaggtattgtccaaggtttctccccatgtgatagtctgaaatatggcctcgtgggaagggaaagacctgaccgtcccccagcccgacacctgtaaagggtctgtgctgaggaggattagtaaaagaggaaggaatgcctcttgcagttgagacaagaggaaggcatctgtctcctgcctgtccctgggcaatggaatgtctcggtataaaacccgattgtatgctccatctactgagatagggaaaaaccgccttagggctggaggtgggacctgcgggcagcaatactgctttgtaaagcattgagatgtttatgtgtatgcatatccaaaagcacagcacttaatcctttacattgtctatgatgccaagacctttgttcacgtgtttgtctgctgaccctctccccacaattgtcttgtgaccctgacacatccccctctttgagaaacacccacagatgatcaataaatactaagggaactcagaggctggcgggatcctccatatgctgaacgctggttccccgggtccccttatttctttctctatactttgtctctgtgtctttttcttttccaaatctctcgtcccaccttacgagaaacacccacaggtgtgtaggggcaacccacccctacatctggtgcccaacgtggaggcttttctctagggtgaaggtacgctcgagcgtggtcattgaggacaagtcgacgagagatcccgagtacatctacagtcagccttacggtaagcttgcgcgctcggaagaagctagggtgataatggggcaaactaaaagtaaaattaaaagtaaatatgcctcttatctcagctttattaaaattcttttaaaaagagggggagttaaagtatctacaaaaaatctaatcaagctatttcaaataatagaacaattttgcccatggtttccagaacaaggaacttcagatctaaaagattggaaaagaattggtaaggaactaaaacaagcaggtaggaagggtaatatcattccacttacagtatggaatgattgggccattattaaagcagctttagaaccatttcaaacagaagaagatagcatttcagtttctgatgcccctggaagctgtttaatagattgtaatgaaaacacaaggaaaaaatcccagaaagaaaccgaaagtttacattgcgaatatgtagcagagccggtaatggctcagtcaacgcaaaatgttgactataatcaattacaggaggtgatatatcctgaaacgttaaaattagaaggaaaaggtccagaattaatggggccatcagagtctaaaccacgaggcacaagtcctcttccagcaggtcaggtgctcgtaagattacaacctcaaaagcaggttaaagaaaataagacccaaccgcaagtagcctatcaatactggccgctggctgaacttcagtatcggccacccccagaaagtcagtatggatatccaggaatgcccccagcaccacagggcagggcgccataccatcagccgcccactaggagacttaatcctatggcaccacctagtagacagggtagtgaattacatgaaattattgataaatcaagaaaggaaggagatactgaggcatggcaattcccagtaacgttagaaccgatgccacctggagaaggagcccaagagggagagcctcccacagttgaggccagatacaagtctttttcgataaaaatgctaaaagatatgaaagagggagtaaaacagtatggacccaactccccttatatgaggacattattagattccattgcttatggacatagactcattccttatgattgggagattctggcaaaatcgtctctctcaccctctcaatttttacaatttaagacttggtggattgatggggtacaagaacaggtccgaagaaatagggctgccaatcctccagttaacatagatgcagatcaactattaggaataggtcaaaattggagtactattagtcaacaagcattaatgcaaaatgaggccattgagcaagttagagctatctgccttagagcctgggaaaaaatccaagacccaggaagtacctgcccctcatttaatacagtaagacaaggttcaaaagagccctaccctgattttgtggcaaggctccaagatgttgctcaaaagtcaattgccgatgaaaaagccggtaaggtcatagtggagttgatggcatatgaaaacgccaatcctgagtgtcaatcagccattaagccattaaaaggaaaggttcctgcaggatcagatgtaatctcagaatatgtaaaagcctgtgatggaatcggaggagctatgcataaagctatgcttatggctcaagcaataacaggagttgttttaggaggacaagttagaacatttggaggaaaatgttataattgtggtcaaattggtcacttaaaaaagaattgcccagtcttaaacaaacagaatataactattcaagcaactacaacaggtagagagccacctgacttatgtccaagatgtaaaaaaggaaaacattgggctagtcaatgtcgttctaaatttgataaaaatgggcaaccattgtcgggaaacgagcaaaggggccagcctcaggccccacaacaaactggggcattcccaattcagccatttgttcctcagggttttcagggacaacaacccccactgtcccaagtgtttcagggaataagccagttaccacaatacaacaattgtccctcaccacaagcggcagtgcagcagtagatttatgtactatacaagcagtctctctgcttccaggggagcccccacaaaaaatccctacaggggtatatggcccactgcctgaggggactgtaggactaatcttgggaagatcaagtctaaatctaaaaggagttcaaattcatactagtgtggttgattcagactataaaggcgaaattcaattggttattagctcttcaattccttggagtgccagtccaagagacaggattgctcaattattactcctgccatatattaagggtggaaatagtgaaataaaaagaataggagggcttgtaagcactgatccaacaggaaaggctgcatattgggcaagtcaggtctcagagaacagacctgtgtgtaaggccattattcaaggaaaacagtttgaagggttggtagacactggagcagatgtctctattattgctttaaatcagtggccaaaaaactggcctaaacaaaaggctgttacaggacttgtcggcataggcacagcctcagaagtgtatcaaagtatggagattttacattgcttagggccagataatcaagaaagtactgttcagccaatgattacttcaattcctcttaatctgtggggtcgagatttattacaacaatggggtgcggaaatcaccatgcccgctccattatatagccccacgagtcaaaaaatcatgaccaagatgggatatataccaggaaagggactagggaaaaatgaagatggcattaaagttccagttgaggctaaaataaatcaagaaagagaaggaatagggtatcctttttaggggcggtcactgtagagcctcctaaacccataccactaacttggaaaacagaaaaaccggtgtgggtaaatcagtggccgctaccaaaacaaaaactggaggctttacatttattagcaaatgaacagttagaaaagggtcacattgagccttcgttctcaccttggaattctcctgtgtttgtaattcagaagaaatcaggcaaatggcatacgttaactgacttaagggctgtaaacgccgtaattcaacccatggggcctctccaacccgggttgccctctccggccatgatcccaaaagattggcctttaattataattgatctaaaggattgcttttttaccatccctctggcagagcaggattgtgaaaaatttgcctttactataccagccataaataataaagaaccagccaccaggtttcagtggaaagtgttacctcagggaatgcttaatagtccaactatttgtcagacttttgtaggtcgagctcttcaaccagtgagagaaaagttttcagactgttatattattcattatattgatgatattttatgtgctgcagaaacgaaagataaattaattgactgttatacatttctgcaagcagaggttgccaatgctggactggcaatagcatctgataagatccaaacctctactccttttcattatttagggatgcagatagaaaatagaaaaattaagccacaaaaaatagaaataagaaaagacacattaaaaacactaaatgattttcaaaaattactaggagatattaattggattcggccaactctaggcattcctacttatgccatgtcaaatttgttctctatcttaagaggagactcagacttaaatagtcaaagaatattaaccccagaggcaacaaaagaaattaaattagtggaagaaaaaattcagtcagcgcaaataaatagaatagatcccttagccccactccaacttttgatttttgccactgcacattctccaacaggcatcattattcaaaatactgatcttgtggagtggtcattccttcctcacagtacagttaagacttttacattgtacttggatcaaatagctacattaatcggtcagacaagattacgaataacaaaattatgtggaaatgacccagacaaaatagttgtccctttaaccaaggaacaagttagacaagcctttatcaattctggtgcatggcagattggtcttgctaattttgtgggacttattgataatcattacccaaaaacaaagatcttccagttcttaaaattgactacttggattctacctaaaattaccagacgtgaacctttagaaaatgctctaacagtatttactgatggttccagcaatggaaaagcagcttacacagggccgaaagaacgagtaatcaaaactccatatcaatcggctcaaagagcagagttggttgcagtcattacagtgttacaagattttgaccaacctatcaatattatatcagattctgcatatgtagtacaggctacaagggatgttgagacagctctaattaaatatagcatggatgatcagttaaaccagctattcaatttattacaacaaactgtaagaaaaagaaatttcccattttatattactcatattcgagcacacactaatttaccagggcctttgactaaagcaaatgaacaagctgacttactggtatcatctgcactcataaaagcacaagaacttcatgctttgactcatgtaaatgcagcaggattaaaaaacaaatttgatgtcacatggaaacaggcaaaagatattgtacaacattgcacccagtgtcaagtcttacacctgcccactcaagaggcaggagttaatcccagaggtctgtgtcctaatgcattatggcaaatggatgtcacgcatgtaccttcatttggaagattatcatatgttcatgtaacagttgatacttattcttattcacatttcatatgggcaacttgccaaacaggagaaagtacttcccatgttaaaaaacatttattgtcttgttttgctgtaatgggagttccagaaaaaatcaaaactgacaatggaccaggatattgtagtaaagctttccaaaaattcttaagtcagtggaaaatttcacatacaacaggaattccttataattcccaaggacaggccatagttgaaagaactaatagaacactcaaaactcaattagttaaacaaaaagaagggggagacagtaaggagtgtaccactcctcagatgcaacttaatctagcactctatactttaaattttttaaacatttatagaaatcagactactacttctgcagaacaacatcttactggtaaaaagaacagcccacatgaaggaaaactaatttggtggaaagataataaaaataagacatgggaaatagggaaggtgataacgtgagggagaggttttgcttgtgtttcaccaggagaaaatcagcttcctgtttggttacccactagacatttgaagttctacaatgaacccatcggagatgcaaagaaaagggcctccacggagagggtaacaccagtcacatggatggataatcctatagaagtatatgttaatgatagtgtatgggtacctggccccatagatgatcgctgccctgccaaacctgaggaagaagggatgatgataaatatttccattgggtatcgttatcctcctatttgcctagggagagcaccaggatgtttaatgcctgcagtccaaaattggttggtagaagtacctactgtcagtcccatcagtagattcacttatcacatggtaagcgggatgtcactcaggccacgggtaaattatttacaagacttttcttatcaaagatcattaaaatttagacctaaagggaaaccttgccccaaggaaattcccaaagaatcaaaaaatacagaagttttagtttgggaagaatgtgtggccaatagtgcggtgatattataaaacaatgaatttggaactattatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccccaagaccaaaaatagtaagtcctgtttctggtcctgaacatccagaattatggaggcttactgtggcctcacaccacattagaatttggtctggaaatcaaactttagaaacaagagattgtaagccattttatactgtcgacctaaattccagtctaacagttcctttacaaagttgcgtaaagcccccttatatgctagttgtaggaaatatagttattaaaccagactcccagactataacctgtgaaaattgtagattgcttacttgcattgattcaacttttaattggcaacaccgtattctgctggtgagagcaagagagggcgtgtggatccctgtgtccatggaccgaccgtgggaggcctcaccatccgtccatattttgactgaagtattaaaaggtgttttaaatagatccaaaagattcatttttactttaattgcagtgattatgggattaattgcagtcacagctacggctgctgtagcaggagttacattgcactcttctgttcagtcagta

SEQ ID NO: 6 - protein, 6 aa, Homo sapiens
PCPVLD

SEQ ID NO: 7 - protein, 6 aa, Homo sapiens
LSPVIH

SEQ ID NO: 8 - DNA, 18 bp, Homo sapiens
ccctgtccagtgttagac

SEQ ID NO: 9 - DNA, 18 bp, Homo sapiens
ctctctccagtgatacat

SEQ ID NO: 10 - DNA, 35 bp, Homo sapiens
tccaggacccctgtccagtgttagacaggagcatg

SEQ ID NO: 11 - DNA, 35 bp, Homo sapiens
tccaggacctctctccagtgatacataggagcatg

SEQ ID NO: 12 - DNA, 21 bp, Homo sapiens
tgaatacattaaccagggggc

SEQ ID NO: 13 - DNA, 21 bp, Homo sapiens
tgactacattaaccagggggc

(19) United States
(12) Patent Application Publication
(10) Pub. No.: US 2023/0175065 A1
(43) Pub. Date: Jun. 8, 2023

(54) METHODS FOR TREATING INFLAMMATORY AND AUTOIMMUNE DISORDERS

(71) Applicant: President and Fellows of Harvard College, Cambridge, MA (US)

(72) Inventors: Nolan KAMITAKI, Cambridge, MA (US); Steven MCCARROLL, Cambridge, MA (US)

(73) Assignee: President and Fellows of Harvard College, Cambridge, MA (US)

(21) Appl. No.: 17/923,872

(22) PCT Filed: May 7, 2021
(86) PCT No.: PCT/US2021/031376

§ 371 Date: Nov. 7, 2022

Related U.S. Application Data
(60) Provisional application No. 63/022,372, filed on May 8, 2020.

(51) Int. Cl. C12Q 1/6883; A61P 37/06; C07K 16/40

(52) U.S. Cl. CPC C12Q 1/6883; A61P 37/06; C07K 16/40; C12Q 2600/118; C12Q 2600/156; C07K 2317/24

(57) ABSTRACT

As described herein, the present invention features compositions and methods for evaluating the propensity of a subject for having or developing an autoimmune and/or inflammatory disease or disorder and for treating autoimmune and inflammatory diseases or disorders.

### CROSS REFERENCE TO RELATED APPLICATION

This international PCT application claims priority to and benefit of U.S. Provisional Application No. 63/022,372, filed on May 8, 2020, the entire contents of which are incorporated by reference herein.

### STATEMENT OF RIGHTS TO INVENTIONS MADE UNDER FEDERALLY SPONSORED RESEARCH

This invention was made with government support under Grant No. HG006855, awarded by the National Human Genome Research Institute and under Grant Nos. MH112491, MH105641, and MH105653 awarded by the National Institute of Mental Health. The government has certain rights in the invention.

### BACKGROUND OF THE INVENTION

Many common illnesses differentially affect men and women for unknown reasons. The autoimmune diseases lupus and Sjögren's syndrome affect nine times more women than men, whereas schizophrenia affects men more frequently and severely.

Likewise, early reports suggest that despite similar rates of infection, men are dying from Covid-19 more often than women, as happened during previous outbreaks of the related diseases SARS and MERS.

Systemic lupus erythematosus (SLE, or “lupus”) is a systemic autoimmune disease of unknown cause. Risk of SLE is heritable (66%), though SLE may have environmental triggers, as its onset often follows events that damage cells, such as infections and severe sunburns. Most SLE patients produce autoantibodies against nucleic acid complexes, including ribonucleoproteins and DNA.

In genetic studies, SLE associates most strongly with variation across the major histocompatibility complex (MHC) locus. However, conclusive attribution of this association to specific genes and alleles has been difficult; the identities of the most likely genetic and allelic culprits have been frequently revised as genetic studies have grown in size. In several other autoimmune diseases, including type 1 diabetes, celiac disease, and rheumatoid arthritis, strong effects of the MHC locus arise from HLA alleles that cause the peptide binding groove of HLA proteins to present a disease-critical autoantigen. In SLE, by contrast, MHC alleles associate broadly with the presence of diverse autoantibodies.

All three illnesses have their strongest common-genetic associations in the Major Histocompatibility Complex (MHC) locus, an association that in lupus and Sjögren's syndrome has long been thought to arise from HLA alleles. Provided herein are compositions and methods that address serious medical needs for treating and diagnosing patients having and at risk for various illnesses, particularly, inflammatory and autoimmune diseases.

### SUMMARY OF THE INVENTION

As described below, the present invention features compositions and methods for treating autoimmune and inflammatory diseases and disorders, as well as infections that may lead to inflammation and other pathologies, such as Covid-19/SARS viral infection.

The invention is based, at least in part, on the discovery that autoimmune disorders, such as systemic lupus erythematosus (SLE/lupus) and Sjögren's syndrome (SjS), which were found to show similar patterns of genetic association at the MHC locus, might also be driven by variation in the complement component 4 (C4) alleles in the Major Histocompatibility Complex (MHC). In accordance with the invention, the C4 genes in the MHC locus generate variation in risk for lupus and for Sjögren's syndrome. In an embodiment, the C4A allele protects more strongly than the C4B in both illnesses.

In an aspect of the invention, a method for evaluating the propensity or risk of a subject for having or developing an autoimmune disease or disorder is provided, in which the method involves detecting in a sample obtained from the subject a dosage of C4A and C4B in the subject's genome, wherein increased dosage of C4A and C4B relative to a reference indicate that the subject has a reduced propensity or risk for having or developing the autoimmune disease or disorder. In an embodiment of the method, for each C4B copy number, a greater C4A copy number is associated with significantly reduced propensity or risk. In an embodiment of the method, for each C4A copy number, a greater C4B copy number is associated with more modestly reduced propensity or risk. In an embodiment, the method further comprises calculating the subject's C4-derived risk score, wherein the risk score is calculated as 2.3 times the number of C4A genes, plus the number of C4B genes, in the subject's genome. In an embodiment of the method, the subject's joint C4A and C4B gene copy number is calculated by summing the C4A and C4B gene contents for each possible pair of two inherited C4 alleles. In an embodiment of the method, the C4 alleles are selected from the group consisting of B(S), A(L), A(L)-B(S)-2, A(L)-B(S)-3, A(L)-B(S)-4, A(L)-B(L)-1, A(L)-B(L)-2, A(L)-A(L)-1, A(L)-A(L)-2, and A(L)-A(L)-3. In an embodiment of the method, the protective effect of the C4A copy number is increased in a male subject relative to a female subject. In an embodiment of the method, the protective effect of the C4A copy number is increased in a subject of European ancestry relative to a subject of African ancestry. In an embodiment of the method, the autoimmune disease is systemic lupus erythematosus or Sjögren's syndrome. In an embodiment of the method, the genome is characterized by whole genome sequencing. In an embodiment of the method, the sample comprises cells, plasma, or cerebral spinal fluid. In an embodiment of the method, calculating the subject's C4-derived risk score and/or joint C4A and C4B gene (allele) copy number is provided by performing computational analysis. In an embodiment of the method, computational analysis and/or an algorithm is applied for facilitating the determination of the subject's propensity or risk.

In an aspect of the invention, a method of treating inflammation in a subject is provided, in which the method involves administering an effective amount of a C4 inhibitor to the subject, thereby treating the inflammation. In an embodiment, the inflammation is associated with a corona virus infection. In an embodiment, the inflammation is associated with Covid19. In an embodiment, the subject is a male. In an embodiment, the effective amount of the C4 inhibitor is increased in a male subject relative to the amount that the C4 inhibitor is increased in a female subject. In an embodiment, the C4 inhibitor is Eculizumab/Soliris, Cetor/Sanquin, an anti-C1q antibody or fragment thereof.

In another aspect of the invention, a method of treating an autoimmune disorder in a subject is provided, in which the method involves administering an effective amount of a C4 agonist, activator, or C4 supplementing agent to the subject, thereby treating the autoimmune disorder. In an embodiment, the autoimmune disorder is systemic lupus erythematosus (SLE). In an embodiment, the autoimmune disorder is Sjögren's syndrome (Sjs). In an embodiment, the subject is female.

In another aspect, a method of pre-selecting a subject for treatment of an autoimmune and/or inflammatory disorder is provided, in which the method comprises detecting in a sample obtained from the subject an alteration in copy number and/or level of a nucleic acid sequence of a C4A and/or C4B polynucleotide or an alteration in the level of a C4A and/or C4B polypeptide encoded by the polynucleotide compared to known levels of the C4A and/or C4B polynucleotide or polypeptide in a control healthy normal subject or in a control subject having an autoimmune and/or inflammatory disorder, thereby pre-selecting the subject for treatment; and administering to the subject a therapeutic amount of an agent to treat the autoimmune and/or inflammatory disorder. In an embodiment, the pre-selected subject has a low copy number or level of the C4A polynucleotide or polypeptide in the sample. In an embodiment, the sample is cerebrospinal fluid (CSF) or plasma. In an embodiment, the autoimmune disorder is systemic lupus erythematosus or Sjögren's syndrome. In an embodiment, the subject is treated with an agent that alters C4 expression or activity. In an embodiment, the agent increases C4 expression or activity. In an embodiment, the subject is male. In an embodiment, the subject is an adult of 20-50 years of age.

Compositions, articles and methods defined by the invention were isolated or otherwise manufactured, or were carried out, in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In some embodiments, the agent is a small molecule chemical compound.

By “alteration” is meant a change (increase or decrease) in the expression levels, copy number, or sequence of a gene or polypeptide as detected by standard art known methods such as those described herein. In some embodiments, an alteration in expression level includes a 10% change in expression levels, a 25% change, a 40% change, and a 50% or greater change in expression levels. In some other embodiments, an alteration in copy number includes an increase or a decrease by at least 1, at least 2, at least 3, at least 4, or at least 5 copies of the gene in a genome. In some embodiments, the alteration in copy number is an increase by at least 1, at least 2, at least 3, at least 4, or at least 5 copies of the gene.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies in the present invention may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antibody specifically binds to C4A polypeptide.

The term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

“Biological sample” as used herein means a biological material isolated from a subject, including any tissue, cell, fluid, or other material obtained or derived from the subject. In some embodiments, the subject is human. The biological sample may contain any biological material suitable for detecting the desired analytes, and may comprise cellular and/or non-cellular material obtained from the subject. In various embodiments, the biological sample may be obtained from the brain. In particular embodiments, the biological sample is blood. In certain embodiments, the biological sample is cerebrospinal fluid (CSF). Biological samples include tissue samples (e.g., cell samples, biopsy samples), such as tissue from the brain. Biological samples also include bodily fluids, including, but not limited to, cerebrospinal fluid, blood, blood serum, plasma, saliva, and urine.

By “capture reagent” is meant a reagent that specifically binds a nucleic acid molecule or polypeptide to select or isolate the nucleic acid molecule or polypeptide.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

A “complement component 4 polypeptide” or “C4 polypeptide” is a complement component 4A (C4A) polypeptide or a complement component 4B (C4B) polypeptide. By “complement component 4A polypeptide” or “C4A polypeptide” is meant a polypeptide or fragment thereof having at least about 85% amino acid identity to GenBank Accession No. AAA51855.1 and having activities that include binding to antigen-antibody complex and binding to other complement components. Human C4 exists as two paralogous genes (isotypes), C4A and C4B; the encoded polypeptides are distinguished at a key site that determines which molecular targets they bind. The sequence of C4A polypeptide provided at GenBank Accession No. AAA51855.1 is shown below:

| | |
|---|---|
| 1 | mrllwgliwa ssfftlslqk prlllfspsv vhlgvplsvg vqlqdvprgq vvkgsvflrn |
| 61 | psrnnvpcsp kvdftlsser dfallslqvp lkdakscglh qllrgpevql vahspwlkds |
| 121 | lsrttniqgi nllfssrrgh lflqtdqpiy npgqrvryrv faldqkmrps tdtitvmven |
| 181 | shglrvrkke vympssifqd dfvipdisep gtwkisarfs dglesnsstq fevkkyvlpn |
| 241 | fevkitpgkp yiltvpghld emqldiqary iygkpvqgva yvrfgllded gkktffrgle |
| 301 | sqtklvngqs hislskaefq dalekinmgi tdlqglrlyv aaaiieypgg emeeaeltsw |
| 361 | yfvsspfsld lsktkrhlvp gapfllqalv remsgspasg ipvkvsatvs spgsvpevqd |
| 421 | iqqntdgsgq vsipiiipqt iselqlsvsa gsphpaiarl tvaappsggp gflsierpds |
| 481 | rpprvgdtln lnlravgsga tfshyyymil srgqivfmnr epkrtltsvs vfvdhhlaps |
| 541 | fyfvafyyhg dhpvanslrv dvqagacegk lelsvdgakq yrngesvklh letdslalva |
| 601 | lgaldtalya agskshkpln mgkvfeamns ydlgcgpggg dsalqvfqaa glafsdgdqw |
| 661 | tlsrkrlscp kekttrkkrn vnfqkainek lgqyasptak rccqdgvtrl pmmrsceqra |
| 721 | arvqqldcre pflsccqfae slrkksrdkg qaglqralei lqeedlided dipvrsffpe |
| 781 | nwlwrvetvd rfqiltlwlp dslttweihg lslsktkglc vatpvqlrvf refhlhlrlp |
| 841 | msvrrfeqle lrpvlynyld knltvsvhvs pveglclagg gglaqqvlvp agsarpvafs |
| 901 | vvptaaaavs lkvvargsfe fpvgdavskv lqiekegaih reelvyelnp ldhrgrtlei |
| 961 | pgnsdpnmip dgdfnsyvrv tasdpldtlg segalspggv asllrlprgc geqtmiylap |
| 1021 | tlaasryldk teqwstlppe tkdhavdliq kgymriqqfr kadgsyaawl srdsstwlta |
| 1081 | fvlkvlslaq eqvggspekl qetsnwllsq qqadgsfqdp cpvldrsmqg glvgndetva |
| 1141 | ltafvtialh hglavfqdeg aeplkqrvea siskansflg ekasagllga haaaitayal |
| 1201 | tltkapvdll gvahnnlmam aqetgdnlyw gsvtgsqsna vsptpaprnp sdpmpqapal |
| 1261 | wiettayall hlllhegkae madqaaawlt rqgsfqggfr stqdtviald alsaywiash |
| 1321 | tteerglnvt lsstgrngfk shalqlnnrq irgleeelqf slgskinvkv ggnskgtlkv |
| 1381 | lrtynvldmk nttcqdlqie vtvkghveyt meanedyedy eydelpakdd pdaplqpvtp |
| 1441 | lqlfegrrnr rrreapkvve eqesrvhytv ciwrngkvgl sgmaiadvtl lsgfhalrad |
| 1501 | lekltslsdr yvshfetegp hvllyfdsvp tsrecvgfea vqevpvglvq pasatlydyy |
| 1561 | nperrcsvfy gapsksrlla tlcsaevcqc aegkcprqrr alerglqded gyrmkfacyy |
| 1621 | prveygfqvk vlredsraaf rlfetkitqv lhftkdvkaa anqmrnflvr ascrlrlepg |
| 1681 | keylimgldg atydleghpq ylldsnswie empserlcrs trqraacaql ndflqeygtq |
| 1741 | gcqv |

By “complement component 4 polynucleotide” or “C4 polynucleotide” is meant a polynucleotide encoding a complement component 4A (C4A) polypeptide or a complement component 4B (C4) polypeptide. By “complement component 4A polynucleotide” or “C4A polynucleotide” is meant a polynucleotide encoding a C4A polypeptide. An exemplary C4A polynucleotide sequence is provided at NCBI Accession No. NG_011638.1 (genomic sequence) and is reproduced below.

| | |
|---|---|
| 1 | tgtcttttgg ggtttgtttt tattctctct ttgagttttg tttccttatg cgcccagtta |
| 61 | cttttgaaaa tgttctgggc agatttgcct agattaataa atgccctcca tgttccaatt |
| 121 | actttttttt ttttgagaca gtgtcttacc ctgtcaccaa gctggagtgc agtggtatga |
| 181 | tcttggctca ctgcaacctc tgcctcctga gttcaagtga ttctcctgcc tcagcctccc |
| 241 | aagtagctgg cattacaggc acctgacacc acgcccagct aatttttttt tttttttttt |
| 301 | ttttgagacg gagtctcgct ctgtcaccca ggctggagtt cagtggcatg atcttggctt |
| 361 | actgcaagct ctgcctcctg ggttcaccca ttctcccgcc tcagcctccc gagtagctgg |
| 421 | gactacaggt gcccgccact atgcctggct aattgttttt ttttttgtat ttttagtaga |
| 481 | gatggggttt caccgtgtta gccaggatgg tcttgatctc cggacctcgt gatccacccg |
| 541 | tctcagcctg ccaaagtgct gggattacag gcatgagcca ccgcatctgg cctatttttg |
| 601 | tatttttaat ggagaccggg tttcatcatg ttggccaggc tggtcttgaa cttgaacttc |
| 661 | tgacctcaag tgatccaccc ttagcgtccc aaagtgctgg gattacaggc atgagccacc |
| 721 | gtgcccggcc ccagttattt ttatttttat tttttgagtt agagtctcac tctgtcaccc |
| 781 | aggctggagc gcagtggcat gatctcggct cacagcaact ttctgggttc aagcagttct |
| 841 | cctgtgtcag cctcctgagt agctgggact acaggcacac atcaccacgc ccggctaatt |
| 901 | tttgtagttt tagtagagac ggggttttac catattggtc aggctgatat tgaactcctg |
| 961 | acctcaggtg atccacccac gtcagcctcc caaagtgccg ggattacagg cttgagccat |
| 1021 | ctcgcccggc ctacttagat gttatattag tggtaattcc tgttatcctg tgagctcttt |
| 1081 | agtgtctaaa caattttttt taagagatgg ggtctcactg tgttgcccag ttgcaatcat |
| 1141 | atcttactgc agcctcaaac tcctgggtca agtgatcctc ttgccttagt ctcccaagta |
| 1201 | gctaggacca taggtgtctg cccccacgcc tggctgtttt tacatttttt gtagagatgt |
| 1261 | ggcgggtggg ggggtctcac tgtgttgccc agactggtct cgaactcctg tcctcaattg |
| 1321 | atcctgctac ctcagcctcc caaaatgctg aattacaggc atgagccact gtacctggtc |
| 1381 | ttaaacaatt ttaaaataac atttttatcc aggattttag ttaattttca acaggtggat |
| 1441 | tagttcttgc tgtattctcg taaacagaag tcctggttta tttttatttg ttttaaacat |
| 1501 | tgaatcccat actcctcccc accttaccct acccagaatt tagactgtta atgttttgaa |
| 1561 | gccacagcct gcatcttaat cactatttta tcttagtgcc tggtcttaga aattatattg |
| 1621 | actctttgat agaccatata taaggcaggt ggatgagaat gtgggtagct agttggaaaa |
| 1681 | ggctgcttgg tcatttgctt gattattttc tcacacagtt tttcctttac taagagaaaa |
| 1741 | tgcccccata ttggcaaaca aaatctccct gcctgagagc gcccagagta tagcagagca |
| 1801 | tcttaccctg atacgcctct tttcactctc ttctctgtgg agacagaagg agcttcaaga |
| 1861 | gcagggggag atcagaatcg tccagctggg cttcgacttg gatgcccatg gaattatctt |
| 1921 | cactgaggac tacaggacca gagtatgtga ctgtgtgcgt caggggtgct ggggggaggg |
| 1981 | cacaggttgg gggagacagg gaacttggga aacagaaata aaaacaaaag aaagaatttc |
| 2041 | cctgccccca catcccatgg agagggcaca gggccctggt aaatagtaat atgagggaga |
| 2101 | gagacaggag ggaaagaggg aggagtgaga gggtaaagag ggggggagag gagggggagg |
| 2161 | aggaggaagg aaggaggggg aggaggaggg ggggaggaag agggggagga ggatgaagag |
| 2221 | gaggaggaag aagaagggta tgagaggtgg aaggatctga gcaagaggta agacaggaag |
| 2281 | agaaatgctg tcctgggggt ggaggttggt agagagtgag ggtggggatg gaccatgtct |
| 2341 | ctcatctctg cttgtaggtc ctcaaggcct gtgatggccg accgtatgct ggggcagtgc |
| 2401 | agaaatttct agcttcagta cttccagcct gtggggacct tagtttccag caggaccaaa |
| 2461 | tgacacagac ctttggcttc agggactcag aaatcacgtg agacttgtgg aaccaaccaa |
| 2521 | agtcaggcat ctggtgcttc cctgcctccc tccagttcca tccagcctgt cctcctgttt |
| 2581 | ttttggtgaa cctgccagaa aagctgccaa aaagctgact cttcttgtta ataaaatgac |
| 2641 | ccaagtttgt attcctcccc acaagagagg aggcctatct tacctgggcc ttagaaagag |
| 2701 | ccctgaaata gaattcagtt cttggtggct tatcaaaagc acacaggggc ctggcaggaa |
| 2761 | gtgtaaaagc ttgatgttaa tcatactggg actaagagga tagagaatgg taggagctgg |
| 2821 | gataccccta aacattcaca ttaaaacaaa aaaaacccaa agctaaaaaa caactgggca |
| 2881 | ggagctaaat aaaaatctaa ttttgagagg ctgtatctgg ctcaggcctc ctactttgta |
| 2941 | acccatggaa tatgtgaaag catttgaaaa actatagcac tgatctcaca tgggcagaca |
| 3001 | cactctcaga gagatgtggt gggagccatg gcgcagtctg cctaggcagt ggcaggagcg |
| 3061 | cagaagactc tgattcctct cctcggtcct aagaccgaat gtgtgtcagg acatgtggtc |
| 3121 | agggaagaga agctatttaa ctgaaccagt aatagtagca ggaaaagaaa aagtggaggg |
| 3181 | agggcagtcc aggtaggggg cctggaacaa gcaactgcac caacagaggc agttggtgcg |
| 3241 | agcacagaac caccccaggc tgggattttg ttatccagtc tctcttgcat ggttgcccgt |
| 3301 | gtttctggag acttgtgtaa acattaatgg atgaggagga gagatggttc tcagagccca |
| 3361 | gccctcatct ctgctggctt cccactgccc tcaggcatct ggtgaatgct ggagtcctca |
| 3421 | ccgtccgaga tgctgggagc tggtggctag ctgtgcctgg agctgggaga ttcatcaagt |
| 3481 | actttgttaa aggtatccca tctgcagctc aagcctgcag cccctcacct tttggtggct |
| 3541 | cctcaggcct ctaggcctta ttcacctttc ccctttcctg tgccacttct cctctagggc |
| 3601 | gccaggctgt ccttagcatg gtccggaagg caaagtaccg ggaactgctc ctatcagagc |
| 3661 | tcctgggccg gcgggcgcct gtcgtggtgc ggcttggcct cacctaccat gtgcacgacc |
| 3721 | tcattggggc ccagctagtg gactggtgag tctttccctg gcctctggca gattatggag |
| 3781 | caatgaccca aagtgggatt tcctcccagc tcatgcttag tttcctagtg aaggccagtg |
| 3841 | gctctcattc ttctctggaa cccgggagca ccccttccca agttctaagt tctcctcaca |
| 3901 | gcttgagcct aggcgtctgg ctccagcctt gtctttctcc tgcacagcat ctctaccact |
| 3961 | tcaggaaccc tcctccgcct gccagagaca tgaagattct gctcatcatt gctcagctcc |
| 4021 | tcagagtggg ccgggagggg actagaagag ctgcatgatg gtggctgaga cagggtcacc |
| 4081 | ttgggaaggc ttgggagcca ggatgagtgt cgggctctcg tgtgtgcaaa aggtcagatg |
| 4141 | tgactgctgc tgtttgcctg gtttctgacc cagtggtggg gtttgagcaa tgcttctctg |
| 4201 | cccttccatg gaaagtggaa ccagaaatgg tgccaaggct gtggctgttc cctttcgtgt |
| 4261 | aaaatggtgc tgttattact ctgtcttgaa ataggaaggt gggatttctg gggaggctgg |
| 4321 | tgaaggaggg cagggttctt ttctctacgt gtcatgttaa aattgccaaa taaagtacct |
| 4381 | ctgcctgtga tattttctgg atgtccttta tttactgtga cgtgtgtttg ggtgccttgt |
| 4441 | ttaggggtag aggtgaagtc tgagctttgc ctcattcaga gaggaaaggg gtcaggggtt |
| 4501 | cactctgacg ttcaggccat tctccctgtg gagtggtgag ggtgtaccta atctcctaaa |
| 4561 | ccacggaatt tctgttaggg cctaaaaaag caaaagccta gtatagttca atttgtgttg |
| 4621 | gaatgaaagt aagagacaag tgtcttagaa gcctgtcatt gttttgtgag ggcctttaaa |
| 4681 | tatcctgtac tcgtgggcca tgttgggccc ttgtacgccc aggtatacat gagcttgtgt |
| 4741 | gcacctatac cctgatacag atatacctgg tagggggagg tgctcaggca ctggaatgag |
| 4801 | aggagttaac ggggaaggac agggttattt ctgggccaag attcagagtt tcccatggac |
| 4861 | acccaggtgt ccggggtgcc cccacaactc tgggcctgag gccagttgca cttcttggct |
| 4921 | gtcacgtggt ttcccagctt agctgggctg ggggaggagc aaggtccaga gtcaactctg |
| 4981 | ccccgaggcc tagcttggcc agaaggtagc agacagacag acggatctaa cctctcttgg |
| 5041 | atcctccagc catgaggctg ctctgggggc tgatctgggc atccagcttc ttcaccttat |
| 5101 | ctctgcagaa gcccaggtcc tggaggcggg atgctgggtg cttggattgg ggcagggctg |
| 5161 | gcatcgggac ccgattcagg agtgagggag agcaggggtg gaggtgtcag agcgaagtct |
| 5221 | gactgctgat cctgtctgtt ctccccaggt tgctcttgtt ctctccttct gtggttcatc |
| 5281 | tgggggtccc cctatcggtg ggggtgcagc tccaggatgt gccccgagga caggtagtga |
| 5341 | aaggatcagt gttcctgaga aacccatctc gtaataatgt cccctgctcc ccaaaggtgg |
| 5401 | acttcaccct tagctcagaa agagacttcg cactcctcag tctccaggta accagacccc |
| 5461 | atgccctcct gctgcttgtg ggggcctcct gccctgttcc catctgtctt gtaagtgtca |
| 5521 | tcatcttccc actggcctcc tcccctcctg tcttcccacc ctggcattct ccttccacgt |
| 5581 | ttctcccttg gtctctgtcc tttttggtca gctgtctctt gctctgtgac ccgctccctc |
| 5641 | tccctctccc tctcctgaca ggtgcccttg aaagatgcga agagctgtgg cctccatcaa |
| 5701 | ctcctcagag gccctgaggt ccagctggtg gcccattcgc catggctaaa ggactctctg |
| 5761 | tccagaacga caaacatcca gggtatcaac ctgctcttct cctctcgccg ggggcacctc |
| 5821 | tttttgcaga cggaccagcc catttacaac cctggccagc ggggtgagtc tcagccccag |
| 5881 | ggcctcaacc tttaaccccc tccgagccct ctcaggatga gtttggtgcc ccctaagtga |
| 5941 | gataacctga aagaaagtgc cacacagaag gggtgcttag gaaacatttg tcccctgctc |
| 6001 | cctctgtgga gtttgaccca ccctcccctt gcacatggac ccctgctcac ctctctcctc |
| 6061 | ctccactccc agttcggtac cgggtctttg ctctggatca gaagatgcgc ccgagcactg |
| 6121 | acaccatcac agtcatggtg gaggtgagtc cccgacctct ggccttcctg atcctggcca |
| 6181 | ctgatgtgac ctcctgcctg tgagcacttc tccccttgca gaactctcac ggcctccgcg |
| 6241 | tgcggaagaa ggaggtgtac atgccctcgt ccatcttcca ggatgacttt gtgatcccag |
| 6301 | acatctcaga gtgagcgctc ccaatgtggg ggctgccccc aagctacacc accccaattc |
| 6361 | ctgttaggct ctccacctcc cacacagagg cacgtcccca gatgccctga ccctcagcct |
| 6421 | cctgagcctc tggttaaccc ccacagtcct cttcccaggg aagcaggctg ctggctctcc |
| 6481 | gtgccccact gtacagatgg gctgagcccc ttccttgtcc attctcaggc cagggacctg |
| 6541 | gaagatctca gcccgattct cagatggcct ggaatccaac agcagcaccc agtttgaggt |
| 6601 | gaagaaatat ggtgagagct ggaaactgga gggacaggca gctgctttcc tgaaggaaat |
| 6661 | aagggtggaa ggagaggtac tgggagcagc tcagggcagg gagatatggg tgccacagcc |
| 6721 | ctgagcagag gggagtcttt gagctggagt ctgacctgcc tatcccttca ccctgggtca |
| 6781 | gtccttccca actttgaggt gaagatcacc cctggaaagc cctacatcct gacggtgcca |
| 6841 | ggccatcttg atgaaatgca gttagacatc caggccaggt aatacctccc tccccacctc |
| 6901 | tgcccaccag caccgggtcc tgctccctac tcagtatgaa tgggctcctg cttccctgcc |
| 6961 | ctcgggccat tattcccccc agcccttggc ccaccctctt ctctctgcca cgacaggtac |
| 7021 | atctatggga agccagtgca gggggtggca tatgtgcgct ttgggctcct agatgaggat |
| 7081 | ggtaagaaga ctttctttcg ggggctggag agtcagacca aggtaggaag gagaataggg |
| 7141 | gctggggagg ggaaggggca agggaggtga ggtgggagac tcagtctcac cctatgtcct |
| 7201 | gtttctttct atgccccagc tggtgaatgg acagagccac atttccctct caaaggcaga |
| 7261 | gttccaggac gccctggaga agctgaatat gggcattact gacctccagg ggctgcgcct |
| 7321 | ctacgttgct gcagccatca ttgagtctcc aggtgggtga ctttccctta ttgtaacccc |
| 7381 | agacccttgc ctctgacctc tgagctaacc ctctgtcctc cggcaccaac accaccccac |
| 7441 | ttctcacatc tcatctcaga ctcaaaacca ggaaacaccc aggagacctg gtttctctcc |
| 7501 | aactctgtct ctgtgactcg gcccttttcc ctggctgagt ttatttattt ctttgctcgt |
| 7561 | tctgctcatt ccttcactcc tccagtggac atgtgttgtt caatgccccg tgctaggcct |
| 7621 | cagcatgcac agacatgttg gggaccagcc tcaacgccac ccgtagggtt cctgaagtcc |
| 7681 | attggtgaca caggaatgag aagagacagg ttaagagttc ataaagagtg ggggccaggg |
| 7741 | ggccaattgc aaaatggagg ctgcaaaagg ctcagagctc tggtctccac actatttttt |
| 7801 | gagtacagtc actcagatct aagaagcaga tgttcaggga gaaacagtga aagggaggca |
| 7861 | gtgggtcata ggcgtaatct atagcaatag agttttaaat gaatctcctt tgtgctcaaa |
| 7921 | cagcatgtct ttaaattatc ggagagtagc tggtggaagt gggcttagct agaagactgc |
| 7981 | atgtctgtcc aatgcttcaa aggagggtct ttctccttga acagagtgtt tacagataag |
| 8041 | acagggggtc tcactctgag catgggaaca tgatggcaat taggaggctt ttcttctcag |
| 8101 | aggcctcttg tggctttcca caacttattg tctcatattt ttatggacag tttatacagg |
| 8161 | caccccacaa gtccttttcc caacatgccc ccctcccttt tttttttttt aaccgctatt |
| 8221 | gctattatgg cttatttgtg gtgtttggtc tgttttcaga agtgtctttt gcatctgtag |
| 8281 | actaaaagta aacagcataa acagatacac attaaagtaa aatttgtaat agttgatcct |
| 8341 | ttaatggtct taatctgttt aagaggattt atgtttgaaa gtccgtcagt agctccaatg |
| 8401 | agaatgtcag tctcaggcag gagggttaaa tgagcctgag atgctttaaa aacctgtttt |
| 8461 | tttaaaattt ggttatattt aatgttaaat ttttattttt ttcttttaga tgatgtctaa |
| 8521 | ctttttaaaa atgatgttta gtagtattat acgaatgggg agttatgtag aaattggaag |
| 8581 | tatttcaatt acattgtact tctaattgat gttttaagtt tattgtacga tcttccattt |
| 8641 | aaataacagt ctgtctaaga tcatttgttt gatttgtcaa ttgttggtct atttgggtct |
| 8701 | gagaattcca caattttgag gaattttttg ttaactattt atatattttg tagtttgaac |
| 8761 | agaggagtgt aaagcaattc cagcagccgc agcagtagct gtgactgcaa taaggcccat |
| 8821 | aagactgtta taagggtaaa aataaatctc tttgttttgg taaacacttt tttttaaaac |
| 8881 | atttttgtga caatatgaat ggaaggagag gctttctaag gtctattgag ggaaaccagt |
| 8941 | atccaaactc ctttcttagt ttttatcagt aacacagatg tttttacacc gaacgtggaa |
| 9001 | ttaatacagg tgaaaaggtg acagttttga caagtaatag tttgagaatt aggtcgaatg |
| 9061 | tcaatatttt tgaccattaa cataaaagga gggttgacac aactctgaat gggcactgtt |
| 9121 | ttgttggaag aaaactgata cgcaaattga agtttttaac cttttttttt taaagataat |
| 9181 | atattttttt ctaaacttaa atatgagatt gggccattat taactttcat aatttggagt |
| 9241 | gtttagggcc tattattgga ttaattattt tgggatgtgg gccagctgta ctaaaattgg |
| 9301 | tccaaattat gggaaaatga gcacgttttt cagtgtaagt agtgttacct ttttgatagt |
| 9361 | atagtttctg ttttagtttt gtcttgtatt tattattttg atgggtacaa ttaactgtaa |
| 9421 | aggtcccctc aggggaccaa ttaatgacaa tttcatagga attattttgt agtaccatag |
| 9481 | tgtgatcaga gatgtaattt tttttaatta atatttttaa attatttgac cattgttaag |
| 9541 | gttgttggca cctctttttt gggggcttaa actgttaatt gaattgaact ctgtgaatga |
| 9601 | tccgggctcc atccagaaaa taaatgatag gatactggtc tttgattatg acctggaatt |
| 9661 | ttaactagtc aatgttgtcg gtagcctttt aggcaaccga tagttggcct tatgtaaaga |
| 9721 | ggggggaact gataacctat ggacacattt attaactttt ttttttttcc tttgggtgag |
| 9781 | agggcccatg agtatttgta ggcttaggga tccaaacgct attattaaca taaacttcaa |
| 9841 | ctgggggttt taaccatgtg acaggcctaa ttaaaggcag gaatgggaca catgcccaat |
| 9901 | aggtataatt ttgggctgtt gtagccacag gtttgttagg cgaggaggtc actgttttta |
| 9961 | ttttggcttt gtattctagg attagtaaat aacagaagac aaacatgagt ataattagta |
| 10021 | actttttttt ttagtaaaag agtgacctgt agtgttactt ggcatcttag tttactatat |
| 10081 | gttattaatg aggaacccca ctgggggtat gttaatttat tctagctaag cagttatgtt |
| 10141 | attagaagct gagaaggggg tgtttgttaa agtaacaggg cagaagaaag gcggatttaa |
| 10201 | gatacgagct taatacagtg tagcaggtat aggtagtagg caaagtgaga gaattaaaaa |
| 10261 | tgaataaatt atttggctta gacttttgtt tttttagtat aatgtctgag gcctgtgttg |
| 10321 | tttgtggaag tcgcattgtt gaggctgtag ttcctgtagg gtctttttta ggctggttca |
| 10381 | aatgtttttt tattttttaa ttttttatcc tttgatgagg atgtagtctt taggctggta |
| 10441 | ctggaaattt taggagtggc gtctgtgtta agagactttt tacaattttt aaagagcagg |
| 10501 | ttagtgtttt aagaaaaact tgtgttttat tttaatgttt agtttataga aaactggatg |
| 10561 | atatcttttt aactttagta aatacgttta cacacggaat tttttacaat tatcatttta |
| 10621 | aaacttgttt agatctttaa aacaaaatta aacaaccttt tttgtataaa ttttttataa |
| 10681 | ctttttttat gacttttaca gacaattttt aacatgtctt aactttttat gttttataat |
| 10741 | ttttttacta aaggtacatt tttataactt tttaaatttt tttacttttt tgtatttttt |
| 10801 | tgatttttgt cttagtcttt tttttacttt tattttttta aatgtgtaat aattagatga |
| 10861 | gtgttggtaa caatggatgt atgtacatat tttagttttt aaaatttagg gatgtgttta |
| 10921 | acatctgttt gccagaactg actaggttcc aattctttac ggttaacacc tattgaagga |
| 10981 | gggtatgtgc ctgtgagctg gtaatctggg cattgtggga taatttgttt agccagcctc |
| 11041 | tgtgtaagtt gaaattattt agataagttt ctccaatttt ggtggaataa tcgatgtgat |
| 11101 | tgggtggctt ggtcaagcag tgatgtcata acctgaaggt ctgcttgatt attgccgtaa |
| 11161 | gccaatgggc caggcagaga gctgtgggct cgaatgtgtg taataaaagt aggatgtgta |
| 11221 | ccttggtcta gtaattgttg aagttgaaga aaaagaccac acagagtggg ctccagagca |
| 11281 | aacttaaggc tgtaatagtt tttaaataaa tacacagaat aaccttagct ctctgaatgt |
| 11341 | tagtaaattc agatcaagtg attggattat gtggtctcca ccagactgtt gctttttcat |
| 11401 | gtttaccaga cccaccagta aaaacagcta tggctccttc caaaggggca tcacaagtaa |
| 11461 | tttttggaag aacctatgta gttaatttta agaattgaaa agtttttagg ataatgatta |
| 11521 | ttaatacatc caacaaattt tgttaaatta atctgtcatg taactgagtt aataaatgcc |
| 11581 | tgtttaacct gatttttatt tattggaact ataattttta ttgggctcag tgccacaaaa |
| 11641 | tttaataatt catatatgag cctgtccaat tagaattgcc atctgattta agtatactgt |
| 11701 | aagtgctttt atggtattat gtggcaaaaa ggaccattta actaaatcat cattttgaac |
| 11761 | aataaccccc attattgtgt ggttagtgtg aagtagggaa cacaatgaat tataaaggca |
| 11821 | agtctgagtc aatcctactg acctgggctt gctgaatttt gttttcaatt actgataact |
| 11881 | ctttcatggc ctcgggtgtt agttctctgt tactgcgtaa gttggtattt cccctcaata |
| 11941 | ttgagaagag attagacata gcataagtag gaattgctaa attgggccaa atccaattaa |
| 12001 | tatcttctaa caatttttga aaattattta aggttttgaa agaatctctt ctaatttgaa |
| 12061 | ccttttgagg cttaatggct ctatcctgta cttgtatttt caaatactga aaaggagtgg |
| 12121 | ttgtttgaat tttgtcaggt gctataagta attcagcatt tgtaattgtc ttttgcaaag |
| 12181 | attaataata ttgaataagt tggtctctac tttttgctgc acaaatctgg aaactgatct |
| 12241 | ctaacaggct ggatagttct gcctacaaaa gtttgacaaa ctgtgggact atttaacata |
| 12301 | ccctggggca aaactttcca atgatatttg gctgcaggtt ttttgttatt aacggcagga |
| 12361 | atggtaaagg caaatttttt gaaatctgcc tctgctaaag gaattgtaaa aaagcagtct |
| 12421 | tttaaatcta taataacaag cggtcagtct ttagggagca cagtggggga tgggagccca |
| 12481 | ggttgtaagg ctcccatcgg ttgaattaca gcgttgacgc catctaccgg actttttctt |
| 12541 | aattacaaat actggggaat tccaaggaga gaaagtgggt gaaatatatc ctttttttag |
| 12601 | tagtttattt tataaagcac ccccaacttt tccttaggga gcggccactg ttcaacccag |
| 12661 | acggggcgcc gggtcatcca ttttaaggga aattgctcct tcactgtaat aactgtaggg |
| 12721 | tgaacctgaa ttgccccatc tccataatga actgtgggtc gggcaataat gggcacggtg |
| 12781 | agccaagtct cgggctccct ccccctgcac ccactcggct gaggaggagg tggccattct |
| 12841 | ggacatttct ctacaggaac cgtgggctga acaatttttt gagtaggttt agggagactg |
| 12901 | gggagattgg cataaatcat cttcagactc tcctttttgt tagtactcgg tagaggtggt |
| 12961 | tcagagttct gattatcaaa ctcctctctc tcctcctctg actcagcctc attatctgtc |
| 13021 | tgaaaaggct ccagtgctgc atgcaccaat gaccaaagcg accaaacagg caaaggaatt |
| 13081 | tcctttcctt ctctatatgc tcttttaagg tcctttccaa ctccttctta atgttttaat |
| 13141 | ttcaaagttt cctgttttgg gaaccaaggg caaaattgtt ccatagcatg aaacaaatcc |
| 13201 | ataagatttt ccgtatcaac ttttacccca ccatgcatgc ttgaagagct gccgtaggaa |
| 13261 | gctcaaatac gtggtgtact tactttcagt ttttcccatt gtgtccctag ctttctctgg |
| 13321 | gcgccccgct tacctgtaga ggttaaaact tttatgtcct tgggagtcct ttgttcgttg |
| 13381 | gtcctctgtt tcacatgctt gagcgtttcc tcaccagatt cttttgggcc ccacgttggg |
| 13441 | cgccagaatg ttggggacca gcctcaacac cacctgtagg gtacctgaag tctggtggtg |
| 13501 | acaaaggaat gagaagagac aggttaagag ttcataaaga gtggaggcca gggggccaat |
| 13561 | tgcaaaatgg aggctgcaaa aggctcagag ctctggtctc cacactattt attgagtaca |
| 13621 | ataacttaga tctaagaagc agatgttcag ggcaaaacag tgaaagggta gcagtgcgtc |
| 13681 | acaggcataa tctacagcag aagcgcttta aatgaatctc ctttgtgctc aaacagcata |
| 13741 | tctttaactt atcggagagt agctagtggg agtgggctta actaggagcc tgcacgtctg |
| 13801 | tccacattcc aatgcttcaa aggagggtct ttctccttga atacagtgtt tacagataag |
| 13861 | agagagcagg tctcgctctg agcatggcaa ttaggaggct tttctcctca gaggcctctt |
| 13921 | gtggctttcc acaacttatt gtcccatatt tttatggcca gtttatacag gcaccccaca |
| 13981 | agtccttttc ccaacacaga caggaatacg gcagcctgtg ccctgggagc tcactgtctt |
| 14041 | gtgggaggga accactcaag ccactcccca cttgtcctcc tgtccctctc ttcttgggct |
| 14101 | ctgtccccca cctctctctg tcctttgtct tgcaggtggg gagatggagg aggcagagct |
| 14161 | cacatcctgg tattttgtgt catctccctt ctccttggat cttagcaaga ccaagcgaca |
| 14221 | ccttgtgcct ggggccccct tcctgctgca ggtttcttcc agaggggaag gatgagtagg |
| 14281 | gaggatgtgg tagttaggag ggctcagggt ctgaccactc tcttttgcct gccctccttt |
| 14341 | acctgcctag gccttggtcc gtgagatgtc aggctcccca gcttctggca ttcctgtcaa |
| 14401 | agtttctgcc acggtgtctt ctcctgggtc tgttcctgaa gtccaggaca ttcagcaaaa |
| 14461 | cacagacggg agcggccaag tcagcattcc aataattatc cctcagacca tctcagagct |
| 14521 | gcagctctca gtaggactcc tcggacccct gggagatggt gggggaaggg gaggagggtg |
| 14581 | agctggggtc ccaaggatcc atggcctgac ttggggggaa ggtggggtac ttggctctga |
| 14641 | gctactaccc tattcgcacc tgaccccctc tccaggtatc tgcaggctcc ccacatccag |
| 14701 | cgatagccag gctcactgtg gcagccccac cttcaggagg ccccgggttt ctgtctattg |
| 14761 | agcggccgga ttctcgacct cctcgtgttg gggacactct gaacctgaac ttgcgagccg |
| 14821 | tgggcagtgg ggccaccttt tctcattact actacatggt gtgcatgagc tggggagtca |
| 14881 | cggagggctg gggtgcaggg aagagccctc tgggtggggc tgggggggtt caaggctgag |
| 14941 | gctgtcccat gaagaggcaa ccactcttgt ccctcccatt cttggcccag atcctatccc |
| 15001 | gagggcagat cgtgttcatg aatcgagagc ccaagaggac cctgacctcg gtctcggtgt |
| 15061 | ttgtggacca tcacctggca ccctccttct actttgtggc cttctactac catggagacc |
| 15121 | acccagtggc caactccctg cgagtggatg tccaggctgg ggcctgcgag ggcaaggtga |
| 15181 | ccggggtcag gagagatggc acttgtgccg agggggttga ggacagggtg attgccaaca |
| 15241 | gggcatggat ttagcttggg ggcagtgagg ataccgggac tgaaggaagc tctcccactc |
| 15301 | tgaccgcccc cacctgccgc ccctgccagc tggagctcag cgtggacggt gccaagcagt |
| 15361 | accggaacgg ggagtccgtg aagctccact tagaaaccga ctccctagcc ctggtggcgc |
| 15421 | tgggagcctt ggacacagct ctgtatgctg caggcagcaa gtcccacaag cccctcaaca |
| 15481 | tgggcaaggt ttgtccagac cctctccaca gctctctcac ccctccatgg ctcatccccc |
| 15541 | tgcttccctg agccttgggc gcagcccctg gatcccactg aggctcccca cagtctcttc |
| 15601 | cccacttggc cctgtggtct ccatctcctg gctctgtatc ctttcctatc cccccatgtg |
| 15661 | ctgccctctc acctgtgccg agtgctcagt cctgcccctc agccacactt ggctcctagc |
| 15721 | attcctgcct ttcttgcagg tctttgaagc tatgaacagc tatgacctcg gctgtggtcc |
| 15781 | tgggggtggg gacagtgccc ttcaggtgtt ccaggcagcg ggcctggcct tttctgatgg |
| 15841 | agaccagtgg accttatcca gaaagagtga gaacagagaa ggaaggggag tgggtggcgg |
| 15901 | gaagataagg aaggaggaag ggcctgaggg gaccagctgg aagagtccgg gcaggaaggg |
| 15961 | ctgggcaggg gaaggggagg aggggaggag gccgagtgcc tgacggctgg actgcagcct |
| 16021 | ttctctctac caggactaag ctgtcccaag gagaagacaa cccggaaaaa gagaaacgtg |
| 16081 | aacttccaaa aggcgattaa tgagaaatgt gagttgcggg tgcctaggca gtagcttggg |
| 16141 | ctctccacct gggatccggg ttgggggtct gcctctctgc ccctcggctc cttgctgaac |
| 16201 | ccacgtgtgg tatttggggc cagagatccg aattccggga ttacgagtgg aaggtgggca |
| 16261 | gctctctcca gcagcctctc ttatgttgct ggtctcaagg ggtcggggcg ggggctgagg |
| 16321 | tgtatgtcct ttttgtcctc tcatgctcac ccccacctgg ccctgcagtg ggtcagtatg |
| 16381 | cttccccgac agccaagcgc tgctgccagg atggggtgac acgtctgccc atgatgcgtt |
| 16441 | cctgcgagca gcgggcagcc cgcgtgcagc agccggactg ccgggagccc ttcctgtcct |
| 16501 | gctgccaatt tgctgagagt ctgcgcaaga agagcaggga caagggccag gcgggcctcc |
| 16561 | aacgaggtga ggggctgggt ggggctaggg cacaggtggc ggcgcttgga aaggcagaac |
| 16621 | ggtcccctcc tcactcccgt ccaccgtggt cccccagccc tggagatcct gcaggaggag |
| 16681 | gacctgattg atgaggatga cattcccgtg cgcagcttct tcccagagaa ctggctctgg |
| 16741 | agagtggaaa cagtggaccg ctttcaaatg tgagagtgtg tgccggcccg gccttttctc |
| 16801 | tgtgctgtgt ctcggggcca gccggggtag acgggccttc tctgcctttc cctacacaga |
| 16861 | ttgacactgt ggctccccga ctctctgacc acgtgggaga tccatggcct gagcctgtcc |
| 16921 | aaaaccaaag gtgatgtcac cctgtctggg cctcaggtga ccctgcttcc atttccctgt |
| 16981 | accccagctc cctgttccct ttgctcttag tgtaggaaga gggtccagtg atctggggag |
| 17041 | gtctgtgcca gcgtgcagct ggcgtgggcc agagggcaga ggcggactga gacagagctg |
| 17101 | ggtcaccccc acccctccct cctgtggccc tgaagctttg atggcccctc tgatctctgc |
| 17161 | ccctgtgccc acgcttcctt tccctcaggc ctatgtgtgg ccaccccagt ccagctccgg |
| 17221 | gtgttccgcg agttccacct gcacctccgc ctgcccatgt ctgtccgccg ctttgagcag |
| 17281 | ctggagctgc ggcctgtcct ctataactac ctggataaaa acctgactgt gaggccccat |
| 17341 | aggagcctga gcatacagga gttgggggag ccagggccca gtgaggggtg gggaggctaa |
| 17401 | ccgggccagg actctggcca tcctcgtttt cctgccctca ggtgagcgtc cacgtgtccc |
| 17461 | cagtggaggg gctgtgcctg gctgggggcg gagggctggc ccagcaggtg ctggtgcctg |
| 17521 | cgggctctgc ccggcctgtt gccttctctg tggtgcccac ggcagccgcc gctgtgtctc |
| 17581 | tgaaggtggt ggctcgaggg tccttcgaat tccctgtggg agatgcggtg tccaaggttc |
| 17641 | tgcagattga ggtgaatgga gcacccctga atataagtcc ccgggccccc agctttgtcc |
| 17701 | tccaccctca gcactctctc tgctggccag gccaggggcc caacacccaa accaatgcct |
| 17761 | tggtctgttc ccatcttcta caattctgat ccaactctgt ccctggagtt gaaactcaaa |
| 17821 | gttctggggg agtctgcgct agcagggcag gctgtagtcc tgtgtgacct cacaaccatg |
| 17881 | ttttccctga gacagaagga aggggccatc catagagagg agctggtcta tgaactcaac |
| 17941 | cccttgggtg agtgaccctc tacctccagc cattggtttc ctaagtgggt acaggtggtg |
| 18001 | ggggatgtgg acagcaggac aggctgccaa cttcccccat ttccccagac caccgaggcc |
| 18061 | ggaccttgga aatacctggc aactctgatc ccaatatgat ccctgatggg gactttaaca |
| 18121 | gctacgtcag ggttacaggt gggagtgccc tttagtccct tcccagtggc caccttcgga |
| 18181 | ttcatgtggg acttgtggat ccctgcttgg tcccactccc cgtgagcctc tgacacagag |
| 18241 | tcctcagacc tccaccctct ccctcccatg tagcctcaga tccattggac actttaggct |
| 18301 | ctgagggggc cttgtcacca ggaggcgtgg cctccctctt gaggcttcct cgaggctgtg |
| 18361 | gggagcaaac catgatctac ttggctccga cactggctgc ttcccgctac ctggacaaga |
| 18421 | cagagcagtg gagcacactg cctcccgaga ccaaggacca cgccgtggat ctgatccaga |
| 18481 | aaggttctgg gtgcaagggc aagcaggagg ggggccagga aaggacagtt actggaagat |
| 18541 | ggacagccca ggaggctaca gagggaaaga aagggggccc etgatgagga tggggagcat |
| 18601 | ggccttgggc tcaaacagca gaagggtgag tgtcacctga gcggccacct ctcctctcca |
| 18661 | aggctacatg cggatccagc agtttcggaa ggcggatggt tcctatgcgg cttggttgtc |
| 18721 | acgggacagc agcacctggt gagcttggga gagtggttcc agggttctga gggggtcagg |
| 18781 | gctggggcag gggtgggaca gagctggtat gatgggaggg tggataacca ggcacctggg |
| 18841 | ggcgtgggca taatgagaag caagtcctta tccccaaccc tcctttcctg ccctccaggc |
| 18901 | tcacagcctt tgtgttgaag gtcctgagtt tggcccagga gcaggtagga ggctcgcctg |
| 18961 | agaaactgca ggagacatct aactggcttc tgtcccagca gcaggctgac ggctcgttcc |
| 19021 | aggacccctg tccagtgtta gacaggagca tgcaggtgcg ggcatgctgg ggctggcccg |
| 19081 | agaagcgcct gtcggaggac tctctttgcc ccttccccct cctgtttgac atcttttctc |
| 19141 | cccttactag gggggtttgg tgggcaatga tgagactgtg gcactcacag cctttgtgac |
| 19201 | catcgccctt catcatgggc tggccgtctt ccaggatgag ggtgcagagc cattgaagca |
| 19261 | gagagtggta agttcagtgg cgtttctgcc ctctgctggc ccccagctct ctcccttttt |
| 19321 | cctcaggaac ccaggggtcc aggcccaaga ccctcctccc gttttcttcc aggaagcctc |
| 19381 | catctcaaag gcaaactcat ttttggggga gaaagcaagt gctgggctcc tgggtgccca |
| 19441 | cgcagctgcc atcacggcct atgccctgac actgaccaag gcgcctgtgg acctgctcgg |
| 19501 | tgttgcccac aacaacctca tggcaatggc ccaggagact ggaggtgagg ggtgaggcgc |
| 19561 | tcctggcagt gagcctgagg cccaggggac cttaggatcc ctgagtgtgc ccagagggag |
| 19621 | aggctggatg aagactcaga ggaggaatga agttataagc aggggtgggt tgggggagac |
| 19681 | tcaggagagc ccagcagggg gtggctaagg gccaggggac caggctcttc tccctgcctt |
| 19741 | cctgtttact cgtggtctcc cttcactttc agataacctg tactggggct cagtcactgg |
| 19801 | ttctcagagc aatgccgtgt cgcccacccc ggctcctcgc aacccatccg accccatgcc |
| 19861 | ccaggcccca gccctgtgga ttgaaaccac agcctacgcc ctgctgcacc tcctgcttca |
| 19921 | cgagggcaaa gcagagatgg cagaccaggc ttcggcctgg ctcacccgtc agggcagctt |
| 19981 | ccaaggggga ttccgcagta cccaagtagg ggccgtcccc gggctctggc gggggtgggt |
| 20041 | agtcctcaga ccaagggctt gcttgagtcc tggctcaacc tccctaggac acggtgattg |
| 20101 | ccctggatgc cctgtctgcc tactggattg cctcccacac cactgaggag aggggtctca |
| 20161 | atgtgactct cagctccaca ggccggaatg ggttcaagtc ccacgcgctg cagctgaaca |
| 20221 | accgccagat tcgcggcctg gaggaggagc tgcaggtgaa ccactccctg gtgaaccact |
| 20281 | ccctcgcctg ggtagccagg acacctgggc ctcgtggcca ggccagaagc cgtccccacc |
| 20341 | ctcccacccg tggaatcccc gcagcacttc ttcctggggt cttcggggga agactgactt |
| 20401 | cctggctgtg tgacctggag ctctgagctt cagttttctc acttgtagag taacatacac |
| 20461 | agagttcacc ctacagggtc gttagaaggc tgaagtgaga taattcatgt gctggtataa |
| 20521 | actttgtgga aatgtgaggt ggggagagga ggtggggctg ttttgaggaa ggagataagt |
| 20581 | tattggagcc gcaaaaacag gtttgcttgt gcccttctaa catcgccttc ccttttctgt |
| 20641 | tgctgaagtt ttccttgggc agcaagatca atgtgaaggt gggaggaaac agcaaaggaa |
| 20701 | ccctgaaggt gagggccagg gaaggggtgg ggccaggcac tggtggagga gagggtgtgg |
| 20761 | agtgagaggc ctgtgggcag aggcacatgg tccggggaag gaggcagaca cctcagggtt |
| 20821 | ggtgtcccgt gcttccgtcc tgggtgtttt tccccctgct tgctttcgct tgctctcccc |
| 20881 | atctctgggt acctgttgtt tcctttaccc gcctcagtgc tggtggctcc gaatcccact |
| 20941 | cctcagccca ggcctcttcc ctgaaccatg ggccccactc gtcccactcc cacagcacct |
| 21001 | cagacgaggc atgtcccaaa gcccttcttc attctgtgtc tcttgtctgg ctggtgggag |
| 21061 | cccctcccag ccaggagccc agccactact ctagaggccg tgttagtggc ccctctccca |
| 21121 | agcctgtcct tatgtcccta gtgactcctc ctctgctccc ctgctgcctg tggcccttgg |
| 21181 | tgctgcatcc tagattctgt gctgagacgg ccttctccct acctggaact tctctctacc |
| 21241 | tcctgtctcc cctgtctgat ccactgtcca cacggcagtg acactgacct tccaaaagcc |
| 21301 | ccagccagat cagccttggg gaaaagtcac tccccgctgc ccacggctca gatggctggg |
| 21361 | cctctgccca cccctccggc cagacagctc tccttgtcta cacagatccc cttgcctttc |
| 21421 | ctgtccttcc ctgcttcttg gcccacagga caagctcttt cttctccttc aagccttggc |
| 21481 | cagaagcctt tcctgagctt ttcagtccag cctcttccca gcacagtctg gagtgttggc |
| 21541 | ctctgggggc aggcccctgc ttctttacct ctctgtctcg cctgacgcct gtggcgaatg |
| 21601 | tggtgccact cgtgtgtgtg gactgtgcag tgacggggag gaaaaggggc tgaaggcctc |
| 21661 | aaatcctgta gcccagggag atgcccttag gtatggcacc agagaggtct gtggcctcac |
| 21721 | atgtcccacg tcctctccct gccccttgct gagccaggtc cttcgtacct acaatgtcct |
| 21781 | ggacatgaag aacacgacct gccaggacct acagatagaa gtgacagtca aaggccacgt |
| 21841 | cgagtacacg agtgagtgtg ggggttggga ggccttgggg ccaggcaggg gctggcgcag |
| 21901 | ggagccgggt ggccatccca gccctcctca caatgcttcc ctgtgcagtg gaagcaaacg |
| 21961 | aggactatga ggactatgag tacgatgagc ttccagccaa ggatgaccca gatgcccctc |
| 22021 | tgcagcccgt gacacccctg cagctgtttg agggtcggag gaaccgccgc aggagggagg |
| 22081 | cgcccaaggt ggtggaggag caggagtcca gggtgcacta caccgtgtgc atctggtggg |
| 22141 | cgccgggagc tgccctgggc caggggaggg agggcaggac ccaggctggg gctgggcttc |
| 22201 | tggagcccgc gcaggcagaa cctggacgac agctcacacg tctccacagg cggaacggca |
| 22261 | aggtggggct gtctggcatg gccatcgcgg acgtcaccct cctgagtgga ttccacgccc |
| 22321 | tgcgtgctga cctggagaag gtgtggtcag ccacccaggg caaccccctc tgtcccaggt |
| 22381 | actgagccct gtcatgtgca gggcctgtga ccaactcccc ttttccacag ctgacctccc |
| 22441 | tctctgaccg ttacgtgagt cactttgaga ccgaggggcc ccacgtcctg ctgtattttg |
| 22501 | actcggtgag tggggagaga tgaggcagga agggactcga tggcaccggg tttactgagt |
| 22561 | atgcgttagg aggtttctca ggagacagct gtgtcagcgg ctggtgctct tgagaacttg |
| 22621 | tgatgtcatc agagagaagg acaagaatgt gagcccgtga gacacagcag agtaaggggc |
| 22681 | agacctgcag gcggcaggga ccgatgccag tcagcaggga ccctcagggt ttgagaggga |
| 22741 | gtctttccta atgctggttt tattcagctt gaggggctgc ctttgttttt ttgttgaact |
| 22801 | tcctatcttt tttttaatat taaagcgtat tttcctttac aaagtgatgg tggccataga |
| 22861 | tgatagttgt atttgtcttt tcacgacctt atttggctaa aatagttatc aaccctctta |
| 22921 | cggctctcaa aacattttta tttatttatt tagtaaagac agggtctcgc tctgttgccc |
| 22981 | aggctggtct tgaactcccg gcctcaagcg atcctctggc ctaggccttt caaagtaccg |
| 23041 | gatttacagg ccagagccac catgcccggc cttcaaaaaa agttttggaa catttactgt |
| 23101 | aacctctggg agaaaatgtg agaaaggtgt ggtggctgtc attagccagc tgtttgtagg |
| 23161 | tcagggagac ccctacccag tgtgtgcaga ggggccagcc cccatcagct ggggaagcct |
| 23221 | ggctgacaca tctgggttga acacaataga aaacacagag ccaacaagat tcccggatag |
| 23281 | ggagctgacg gtgcagcagc ctagctcagg agggacactg gcacggcacc gtgtggactg |
| 23341 | ggcccgcgtg ggcacgagga ggggtcaggc ctgggacctg agtcgggggg tcaggcagga |
| 23401 | tgacagaacc tgcagttagg ttgtggcaaa taaaggagga cccagttgta tccatgacaa |
| 23461 | agatgaggcc gcgaggaggg cgagtgggtt tgggggcagg cagagtgcct tggagaactt |
| 23521 | acaggtcctg ccacaatcct aatgcaagga tggagctgca agttcagttt gggaatcatc |
| 23581 | agcctggatt ggtttggtgg aagccaggga gtggttgaga cccccacagg ggagctctga |
| 23641 | ggaaggaagt tccgaaggag ggaacgtaag aaatgaccag gtcagaacca agggtggtcc |
| 23701 | agaagctaac ccttagctta gggacagttt cacagagaac acgtccatga tgcaagactc |
| 23761 | tgctgagggc ctggagcagt gaagactggg gcaaggtcac cctctgggaa gtgaagtcac |
| 23821 | cagagacctt gcggagcagc tttgagagtt ctctgagtag gaaggtaaca gaatgtgaag |
| 23881 | gacactggag agaaggccaa taggaagcaa acaaaaacag gccaaggaaa cccagtacag |
| 23941 | ggggctgcag ggcccaggga gtgggtccct catctctcct ccccacgctt ggccaggtcc |
| 24001 | ccacctcccg ggagtgcgtg ggctttgagg ctgtgcagga agtgccggtg gggctggtgc |
| 24061 | agccggccag cgcaaccctg tacgactact acaaccccgg tgagcactgc aggacaccct |
| 24121 | gaaattcagg agaactttgg cataggtgcc ctcctatggg acaatggaca ccggggtagt |
| 24181 | gagggggcag agagccctgg ggctccctgg gactgaggag gcagaatgga ggggcctgtg |
| 24241 | ccctaactcc tctctgttct ccagagcgca gatgttctgt gttttacggg gcaccaagta |
| 24301 | agagcagact cttggccacc ttgtgttctg ctgaagtctg ccagtgtgct gagggtgaga |
| 24361 | ctgagggcct ggggcggggc agtggaggcg ggatggccgg ggcccccccc acactgtctg |
| 24421 | atgggttccc caacttcagg gaagtgccct cgccagcgtc gcgccctgga gcggggtctg |
| 24481 | caggacgagg atggctacag gatgaagttt gcctgctact acccccgtgt ggagtacggt |
| 24541 | cagtcttccc accgaggccc tggcctgacc ctccctcggg gaccggccgt tttggtctct |
| 24601 | ctgggtgtag cctgctcctc ttacaggtca tgcacgcagc ctgtttgctc tgacaccaac |
| 24661 | ttcctaccct ctcagcctca aagtaactca cctttccccc ttctcctcac cccctcttag |
| 24721 | gcttccaggt taaggttctc cgagaagaca gcagagctgc tttccgcctc tttgagacca |
| 24781 | agatcaccca agtcctgcac ttcagtatga agcaaaccgg agaggcgggc agggctgggg |
| 24841 | ggagacaggg aggctgaggt gtggccgagg acctgaccat ctggaagtgt gaaaatcccc |
| 24901 | ttgggctgtc agaagccttg ggcttggcca taaataggga ggcagtggca cctctccatg |
| 24961 | ggggtggcga aggtggaatg agaggatcta cacagagtcc ccagcctggg ctcaccctgc |
| 25021 | accttctctt cccctctgac cacttttgcg cacgtcatcc ccgcagccaa ggatgtcaag |
| 25081 | gccgctgcta atcagatgcg caacttcctg gttcgagcct cctgccgcct tcgcttggaa |
| 25141 | cctgggaaag aatatttgat catgggtctg gatggggcca cctatgacct cgagggacag |
| 25201 | tgagtcatct ggtcccctca gtctcttgtc ctccccatgc ctcgccacct aggccttgcc |
| 25261 | cctcagaagc cagatgcctg tgctctccgt ttccacctgc catcctcccg agccctgctg |
| 25321 | actgcccctt tgccccctgc agcccccagt acctgctgga ctcgaatagc tggatcgagg |
| 25381 | agatgccctc tgaacgcctg tgccggagca cccgccagcg ggcagcctgt gcccagctca |
| 25441 | acgacttcct ccaggagtat ggcactcagg ggtgccaggt gtgagggctg ccctcccacc |
| 25501 | tccgctggga ggaacctgaa cctgggaacc atgaagctgg aagcactgct gtgtccgctt |
| 25561 | tcatgaacac agcctgggac cagggcatat taaaggcttt tggcagcaaa gtgtcagtgt |
| 25621 | tggcagtgaa gtgtcagtgt gtgttgctag ggctgagagc agtgcccctg cccgatgcag |
| 25681 | ttctgggcag gccaggttga cataacctta gactctctga gccctgatga cccttgggct |
| 25741 | gttcagctct gctagaacct cccagatgac ccgctaggag tctagtgctt cacaggacca |
| 25801 | ccccgagcag aactgggacc caagagcctg caccccaagg accagagtcc atgccaagac |
| 25861 | cacccttcag cttccaaggc cctccactgc ccggctgtcg ccagtcacca cggcctcaga |
| 25921 | cagggcttgt gctcagctga cacctgtgac acagctcttc tgcctcatga gctgttgtcc |
| 25981 | agctacacct ccccgactct gtcctcgtgc tgctggcggt tctgaggtct gcagatttta |
| 26041 | gctgagttcc gggctgttga aagcctgctg acgcttggtt ctgttatcag tggaatgagg |
| 26101 | tgactttccc ggagttgtgc aatcctcagg tccggcagtg tcttcttcca gttactggtt |
| 26161 | tcaaacaagc caaaagtctg actttggtgt gtttgtgaat cctctgagga agccgctgtt |
| 26221 | ctcctggggt ctccccttcc caccggacct gcctaacttt cccccattta gtggcacacc |
| 26281 | tggggtcttc agagatgact ccgcgtctgt ccaaagaagt ttggtgagat cagtttccgt |
| 26341 | agaggtcatg acagttcagc agcctgccat ccagtcattc gacagaaatt cgggaatctt |
| 26401 | tcacttcatg ccatgccctg tgccaggtgc cagagataca gctgctcact ccagggctca |
| 26461 | tcgctgggga gacagataag aggacgggca gtccccaccc tctgtgaaag atgtgatgtc |
| 26521 | agggagcagt gtggtcctgt ggggcatcta accaagtcag gggcattgcc aggcagggac |
| 26581 | agggaaggct tcctggagca ggtggcctcc aagtggggct ctgaagactg agaaggagcc |
| 26641 | aggaaaagag caggggtaga tgagggcatc tggggcagaa ggagaatata caaaggccca |
| 26701 | gaggccgggg gcaggacagg gtacctttgg ggacattgca tgtaattgac cacattcgga |
| 26761 | gtttggattt ggaagtggtg gaagagatgg agatggtgag acaagtagta agcacgtcag |
| 26821 | ccttccaggt gcgctccttt ccgatgagca ctgtcttatc ccacgtaact ttgagaagtt |
| 26881 | tgggcctttc ccactgtggc agaggtttcc tgaggctctt gcatacatgg ccctatggtt |
| 26941 | gctcatcaga tctttctccc agtagctgct cagcatggtg gtggcataag cccattttcc |
| 27001 | ggagccaggg attcagttgc agcaagacct ggcccggtct gggaggtcaa ccatgaagaa |
| 27061 | ggcagtagct gtcattgccc aaccccagaa atcccaatcc tgttttctcc ctctcagtcc |
| 27121 | tgatcatgga ttcagcagca gcgaactcgc caatgtagtg ggtggcacag ccagggtctt |
| 27181 | gactctggct ctgcagtagc acagtctgga aaagctctga ggggagagag acccccactg |
| 27241 | gtccgagggt ctggcacaga gccagaaatg ggggggaagg tatggggctg ggtcgcctct |
| 27301 | gacctctcag gtaccatcca ggaggccctg gcctctcact gaacccggcc actcctcttt |
| 27361 | ggcatggcct cttcccaaat ccccaaactg cctccttact cacaaaagtg gtctctgagt |
| 27421 | gtcagtccag tgggaccccc accccttatg gcttcagttc cccaaatagg gctggaccct |
| 27481 | tgatcctgat ccagctgtgg ctatccagcc ccttcctggg gactttggac tttgaggggg |
| 27541 | ggcatgccca gttgtgctgg gaatccatac tttccctggc tggagtagaa cctgtggact |
| 27601 | gtagtcctga gggcagtcat gttc |

By “complement component 4B polypeptide” or “C4B polypeptide” is meant a polypeptide or fragment thereof having at least about 85% amino acid identity to NCBI Accession No. NP_001002029.3 and having activities that include binding to antigen-antibody complex and binding to other complement components. The sequence at NCBI Accession No. NP_001002029.3 is shown below:

| | |
|---|---|
| 1 | mrllwgliwa ssfftlslqk prlllfspsv vhlgvplsvg vqlqdvprgq vvkgsvflrn |
| 61 | psrnnvpcsp kvdftlsser dfallslqvp lkdakscglh qllrgpevql vahspwlkds |
| 121 | lsrttniqgi nllfssrrgh lflqtdqpiy npgqrvryrv faldqkmrps tdtitvmven |
| 181 | shglrvrkke vympssifqd dfvipdisep gtwkisarfs dglesnsstq fevkkyvlpn |
| 241 | fevkitpgkp yiltvpghld emqldiqary iygkpvqgva yvrfgllded gkktffrgle |
| 301 | sqtklvngqs hislskaefq daleklnmgi tdlqglrlyv aaaiiespgg emeeaeltsw |
| 361 | yfvsspfsld lsktkrhlvp gapfllqalv remsgspasg ipvkvsatvs spgsvpevqd |
| 421 | iqqntdgsgq vsipiiipqt iselqlsvsa gsphpaiarl tvaappsggp gflsierpds |
| 481 | rpprvgdtln lnlravgsga tfshyyymil srgqivfmnr epkrtltsvs vfvdhhlaps |
| 541 | fyfvafyyhg dhpvanslrv dvqagacegk lelsvdgakq yrngesvklh letdslalva |
| 601 | lgaldtalya agskshkpln mgkvfeamns ydlgcgpggg dsalqvfqaa glafsdgdqw |
| 661 | tlsrkrlscp kekttrkkrn vnfqkainek lgqyasptak rccqdgvtrl pmmrsceqra |
| 721 | arvqqpdcre pflsccqfae slrkksrdkg qaglqralei lqeedlided dipvrsffpe |
| 781 | nwlwrvetvd rfqiltlwlp dslttweihg lslsktkglc vatpvqlrvf refhlhlrlp |
| 841 | msvrrfeqle lrpvlynyld knltvsvhvs pveglclagg gglaqqvlvp agsarpvafs |
| 901 | vvptaatavs lkvvargsfe fpvgdavskv lqiekegaih reelvyelnp ldhrgrtlei |
| 961 | pgnsdpnmip dgdfnsyvrv tasdpldtlg segalspggv asllrlprgc geqtmiylap |
| 1021 | tlaasryldk teqwstlppe tkdhavdliq kgymriqqfr kadgsyaawl srgsstwlta |
| 1081 | fvlkvlslaq eqvggspekl qetsnwllsq qqadgsfqdl spvihrsmqg glvgndetva |
| 1141 | ltafvtialh hglavfqdeg aeplkqrvea siskassflg ekasagllga haaaitayal |
| 1201 | tltkapadlr gvahnnlmam aqetgdnlyw gsvtgsqsna vsptpaprnp sdpmpqapal |
| 1261 | wiettayall hlllhegkae madqaaawlt rqgsfqggfr stqdtviald alsaywiash |
| 1321 | tteerglnvt lsstgrngfk shalqlnnrq irgleeelqf slgskinvkv ggnskgtlkv |
| 1381 | lrtynvldmk nttcqdlqie vtvkghveyt meanedyedy eydelpakdd pdaplqpvtp |
| 1441 | lqlfegrrnr rrreapkvve eqesrvhytv ciwrngkvgl sgmaiadvtl lsgfhalrad |
| 1501 | lekltslsdr yvshfetegp hvllyfdsvp tsrecvgfea vqevpvglvq pasatlydyy |
| 1561 | nperrcsvfy gapsksrlla tlcsaevcqc aegkcprqrr alerglqded gyrmkfacyy |
| 1621 | prveygfqvk vlredsraaf rlfetkitqv lhftkdvkaa anqmrnflvr ascrlrlepg |
| 1681 | keylimgldg atydleghpq ylldsnswie empserlcrs trqraacaql ndflqeygtq |
| 1741 | gcqv |

By “complement component 4B polynucleotide” or “C4B polynucleotide” is meant a polynucleotide encoding a C4B polypeptide. An exemplary C4B polynucleotide sequence is provided at NCBI Accession No. NG_011639.1 (genomic sequence) and is reproduced below.

| | |
|---|---|
| 1 | atggtgctgg tcctggaggc accggctccg ttctgcatct cctccccgca gtccctgggg |
| 61 | aaggggatcc gcagcccacc tgggagagga gagcaggggc cagtcctttt ccaagcctta |
| 121 | ggccctggct gcccacccag cccccggccc cgggcccgtg cgtccaggta cccgtggtga |
| 181 | aagaggtgga cacgggcggc aggaggctct ggccccacat ggcctggagc cgtgcattgt |
| 241 | aggaggtgga gggaaagagg ccaaggagct ggtgagatgt gatccctcct gggagcagga |
| 301 | tctcctgtgg gacagacaag ggggggtcag gggagaggga ggtggagacc ctccgggagg |
| 361 | gccagaggca gcacctcctg gaatcaccca gggaggggag ttgggtcagt ggggccgggg |
| 421 | cacctggttc tgtccaccag gggtgtggaa gctgagcagg tagcctgcgg gccggactgg |
| 481 | gggctcagtc caagtgagca gggcggtgcg gggggtcact tccttggcct ccaagtcccg |
| 541 | aggggcctct agccctagga gggaaagcag gaagaggaga tggggatgag gcccaacctg |
| 601 | gctccctcta cctcctctcc ctgtcccaca caccccacag accctacctg tggtgaaggt |
| 661 | gatgctggct ggggaagtga ggttggggcc ccgcaggcca cgcactgtgg cggtgtagtt |
| 721 | ggtgtggagg acaaggtcat gcagggggta gtccaccgcg ctgcctgggg tctccgcctg |
| 781 | cagaggcggg gctgggagtg tagagagggg catcaaggcc tgccccctcc atcctcggcc |
| 841 | agagtccagc ctcccccctg caatccccac cctgaacaag tcccctccag aggcctcagg |
| 901 | cctgctcacc cccaggggct gtgacctgga cgtcataggt gtccacagga ttctgggggg |
| 961 | gcttccagtg cagcacggcg aatccctcgg tcaagttcag tgcacgcaac tgtgtgggac |
| 1021 | cgtcaggaac tgggggaagg ggaggggctc agaagggtcc ccgcggctct ctctactccg |
| 1081 | tgcctcccca gactccactg gcctcccgtc cgcaatcgga gcctccacca cctccctttc |
| 1141 | accctcctcg ttctctctca actcccaccc atgccgtttt cttgactccc acctggagtt |
| 1201 | tctgggtccg ggcccggccg tccacctgca cactctgagg ctcccctgaa aacgttgggg |
| 1261 | atcgagggtt acccagggaa ccccagggcg gctggagggt gggcagagtg caggggggag |
| 1321 | aggaaatgcg aggcgatgag cacatggcaa aggcaccacc tccgtccgcc agctggtagg |
| 1381 | agactttgaa gctgtccgcc cgggatggtg ggggcatcca gttgaccttg gctgaggtct |
| 1441 | ccctgatttc actgaattgg aggtcacggg ggctctccag aactgcagag gggtcaagga |
| 1501 | acaatgacgc aggcaggggc agggaggctc ctccctgcga gtccccccct cgcctctgct |
| 1561 | ccagcacagg ctcaccaccc cttttcctct agtccccagg aatggaagtc gctctgcaga |
| 1621 | ttcctccagg cccaccacca actcgcccac ccccaccgct ggctgaggca ctaggtcccc |
| 1681 | cccgtgaagt acaaagaccc ccactttggg gcagagtgtg tgtgggtcct tacctgggct |
| 1741 | gagggtgcgg gcggttccct ggatgctgtc ggccttgtgg ggtcctcgca gcccatacag |
| 1801 | tgtcaggctg tacagagtcc cggaacgcag gtcccggagc acggccgagt gccgcgtccc |
| 1861 | cggcaccatc agctcgcgct gcagcagtgg acgcggatgc ggctccagag tgcttggtga |
| 1921 | tggaacccca aagcggagca ggaaggagtc gaaggccccc ggtggggcct cccagttgag |
| 1981 | cctcagtgaa ctggtggtca cgtcagtcac agacagctgg gacaggcggg gccttgactc |
| 2041 | ctctgaggtc tgaccagcag gagccagccc tgcacggagt gggtggggga gaagggattg |
| 2101 | gagacagaag cacaccagct tggtgaccca gagcacgtcc cttccacccc cctccctgcc |
| 2161 | cccgtttctc tatctgtaac cagggacttg cagccacagg ggggtcctgt ggggcagagc |
| 2221 | taaaggccac tcgcatccag cccatccatc ctctctccct ggtacccgcc tcacgctctt |
| 2281 | tccctgcgac caccccttct gagcccccgt ttctcccttc tgagtcctag gctagaggcc |
| 2341 | ggagacgcct ggtggtacct gtggtgccct cagctgagag gggccccagg cgcttccctt |
| 2401 | catggaggcc atagaggagg aacctgtagc gggtgctggg ctccaggcct gagatgagga |
| 2461 | tcttgctctg gtcgccgtcc acgagcaagg cctggggctg cccattcgtg tcctcatact |
| 2521 | ggaccacgaa ggaatcaaag gggccctggg ccacgctcca cgagaggcgc atggagtctg |
| 2581 | gggttgtgtc ggtcacggtc agcactccta ggcggggctc ttcaggaggc tcaggggcct |
| 2641 | ctggggctaa ctctggggct ggtgtgtcct cttctggggc tgcgtgggag aagcccaggg |
| 2701 | gagaatctga gtgaggggcg ccatggggtg ctccattttt atcttccagg cttggcccaa |
| 2761 | ggctgaggtg ggaagtttat aggtccaggc ccagtcagac aatgaagtcg ctgtggcctc |
| 2821 | gtgactcctg cgagctcccg cgctgtctga gtcaggtgct cgcttccccc ttccacaccc |
| 2881 | cggtgtcctg ccgagcccac ctcgagatat cacaggctct ggccccaccc atgccgggat |
| 2941 | acattcactg agcttgagga gtgtggtgct cccttctgag agaagctgag ggtggaactg |
| 3001 | gctggttgag gtgactggca aatcccacca gccgtgccgt ggtcaggcct gtctgaggtg |
| 3061 | ggcatcagcg agctctggaa gaggagcctg taccacaaat gcagccactg ctgttggttt |
| 3121 | ctgtgtcccc gctcattttg ttttccagtg atgttcctct taagaaaatg ctcctgactc |
| 3181 | atccacggca gggaggtttg ccactatctg gacaaggcca cccttcgggg aggcgacagc |
| 3241 | agccccagcg agtaatgagg agcagcggca gtgacggggc agagtcgggg ctgggagatt |
| 3301 | agagagcccc tcccagggcc tttccctccc gcctggcctg gctcctgctc tggactcctt |
| 3361 | gatggatgtt gaagcccaca gggctgcaga ctcctcctcc ttcctgggca caggccaggt |
| 3421 | caccccactc cggcctgccc actcctgcag tcatctttgt cttcagacca aatgcacaag |
| 3481 | tactttgtta aaggtatccc atctgcagct caagcctgca gcccctcacc ttttggtggc |
| 3541 | tcctcaggcc tctaggcctt attcaccttt cccctttcct gtgccacttc tcctctaggg |
| 3601 | cgccaggctg tccttggcat ggtccggaag gcaaagtacc gggagctgct cctatcagag |
| 3661 | ctcctgggcc ggcgggtgcc tgtcgtggtg cggcttggcc tcacctacca tgtgcacgac |
| 3721 | ctcattgggg cccagctagt ggactggtga gtctttccct ggcctctggc agattatgga |
| 3781 | gcaatgaccc aaagtgggat ttcctcccag ctcatgctta gtttcctagt gaaggccagt |
| 3841 | ggctctcatt cttctctgga acccgggagc accccttccc aagttctaag ttctcctcac |
| 3901 | agcttgagcc taggcgtctg gctccagcct tgtctttctc ctgcacagca tctctaccac |
| 3961 | ttcaggaacc ctcctccgcc tgccagagac atgaagattc tgctcatcat tgctcagctc |
| 4021 | ctcagagtgg gccgggaggg gactagaaga gctgcatgat ggtggctgag acagggtcac |
| 4081 | cttgggaagg cttgggagcc aggatgagtg tcgggctctc gtgtgtgcaa aaggtcagat |
| 4141 | gtgactgctg ctgtttgcct ggtttctgac ccagtggtgg ggtttgagca atgcttctct |
| 4201 | gcccttccat ggaaagtgga accagaaatg gtgccaaggc tgtggctgtt ccctttcgtg |
| 4261 | taaaatggtg ctgttattac tctgtcttga aataggaagg tgggatttct ggggaggctg |
| 4321 | gtgaaggagg gcagggttct tttctctacg tgtcatgtta aaattgccaa ataaagtacc |
| 4381 | tctgcctgtg atattttctg gatgtccttt atttactgtg acgtgtgttt gggtgccttg |
| 4441 | tttaggggta gaggtgaagt ctgagctttg cctcattcag agaggaaagg ggtcaggggt |
| 4501 | tcactctgac gttcaggcca ttctccctgt ggagtggtga gggtgtacct aatctcctaa |
| 4561 | accacggaat ttctgttagg gcctaaaaaa gcaaaagcct agtatagttc aatttgtgtt |
| 4621 | ggaatgaaag taagagacaa gtgtcttaga agcctgtcat tgttttgtga gggcctttaa |
| 4681 | atatcctgta ctcgtgggcc atgttgggcc cttgtacgcc caggtataca tgagcttgtg |
| 4741 | tgcacctata ccctgataca gatatacctg gtagggggag gtgctcaggc actggaatga |
| 4801 | gaggagttaa cggggaagga cagggttatt tctgggccaa gattcagagt ttcccatgga |
| 4861 | cacccaggtg tccggggtgc ccccacaact ctgggcctga ggccagttgc acttcttggc |
| 4921 | tgtcacgtgg tttcccagct tagctgggct gggggaggag caaggtccag agtcaactct |
| 4981 | gccccgaggc ctagcttggc cagaaggtag cagacagaca gacggatcta acctctcttg |
| 5041 | gatcctccag ccatgaggct gctctggggg ctgatctggg catccagctt cttcacctta |
| 5101 | tctctgcaga agcccaggtc ctggaggcgg gatgctgggt gcttggattg gggcagggct |
| 5161 | ggcatcggga cccgattcag gagtgaggga gagcaggggt ggaggtgtca gagcgaagtc |
| 5221 | tgactgctga tcctgtctgt tctccccagg ttgctcttgt tctctccttc tgtggttcat |
| 5281 | ctgggggtcc ccctatcggt gggggtgcag ctccaggatg tgccccgagg acaggtagtg |
| 5341 | aaaggatcag tgttcctgag aaacccatct cgtaataatg tcccctgctc cccaaaggtg |
| 5401 | gacttcaccc ttagctcaga aagagacttc gcactcctca gtctccaggt aaccagaccc |
| 5461 | catgccctcc tgctgcttgt gggggcctcc tgccctgttc ccatctgtct tgtaagtgtc |
| 5521 | atcatcttcc cactggcctc ctcccctcct gtcttcccac cctggcattc tccttccacg |
| 5581 | tttctccctt ggtctctgtc ctttttggtc agctgtctct tgctctgtga cccgctccct |
| 5641 | ctccctctcc ctctcctgac aggtgccctt gaaagatgcg aagagctgtg gcctccatca |
| 5701 | actcctcaga ggccctgagg tccagctggt ggcccattcg ccatggctaa aggactctct |
| 5761 | gtccagaacg acaaacatcc agggtatcaa cctgctcttc tcctctcgcc gggggcacct |
| 5821 | ctttttgcag acggaccagc ccatttacaa ccctggccag cggggtgagt ctcagcccca |
| 5881 | gggcctcaac ctttaacccc ctccgagccc tctcaggatg agtttggtgc cccctaagtg |
| 5941 | agataacctg aaagaaagtg ccacacagaa ggggtgctta ggaaacattt gtcccctgct |
| 6001 | ccctctgtgg agtttgaccc accctcccct tgcacatgga cccctgctca cctctctcct |
| 6061 | cctccactcc cagttcggta ccgggtcttt gctctggatc agaagatgcg cccgagcact |
| 6121 | gacaccatca cagtcatggt ggaggtgagt ccccgacctc tggccttcct gatcctggcc |
| 6181 | actgatgtga cctcctgcct gtgagcactt ctccccttgc agaactctca cggcctccgc |
| 6241 | gtgcggaaga aggaggtgta catgccctcg tccatcttcc aggatgactt tgtgatccca |
| 6301 | gacatctcag agtgagcgct cccaatgtgg gggctgcccc caagctacac caccccaatt |
| 6361 | cctgttaggc tctccacctc ccacacagag gcacgtcccc agatgccctg accctcagcc |
| 6421 | tcctgagcct ctggttaacc cccacagtcc tcttcccagg gaagcaggct gctggctctc |
| 6481 | cgtgccccac tgtacagatg ggctgagccc cttccttgtc cattctcagg ccagggacct |
| 6541 | ggaagatctc agcccgattc tcagatggcc tggaatccaa cagcagcacc cagtttgagg |
| 6601 | tgaagaaata tggtgagagc tggaaactgg agggacaggc agctgctttc ctgaaggaaa |
| 6661 | taagggtgga aggagaggta ctgggagcag ctcagggcag ggagatatgg gtgccacagc |
| 6721 | cctgagcaga ggggagtctt tgagctggag tctgacctgc ctatcccttc accctgggtc |
| 6781 | agtccttccc aactttgagg tgaagatcac ccctggaaag ccctacatcc tgacggtgcc |
| 6841 | aggccatctt gatgaaatgc agttagacat ccaggccagg taatacctcc ctccccacct |
| 6901 | ctgcccacca gcaccgggtc ctgctcccta ctcagtatga atgggctcct gcttccctgc |
| 6961 | cctcgggcca ttattccccc cagcccttgg cccaccctct tctctctgcc acgacaggta |
| 7021 | catctatggg aagccagtgc agggggtggc atatgtgcgc tttgggctcc tagatgagga |
| 7081 | tggtaagaag actttctttc gggggctgga gagtcagacc aaggtaggaa ggagaatagg |
| 7141 | ggctggggag gggaaggggc aagggaggtg aggtgggaga ctcagtctca ccctatgtcc |
| 7201 | tgtttctttc tatgccccag ctggtgaatg gacagagcca catttccctc tcaaaggcag |
| 7261 | agttccagga cgccctggag aagctgaata tgggcattac tgacctccag gggctgcgcc |
| 7321 | tctacgttgc tgcagccatc attgagtctc caggtgggtg actttccctt attgtaaccc |
| 7381 | cagacccttg cctctgacct ctgagctaac cctctgtcct ccggcaccaa caccacccca |
| 7441 | cttctcacat ctcatctcag actcaaaacc aggaaacacc caggagacct ggtttctctc |
| 7501 | caactctgtc tctgtgactc ggcccttttc cctggctgag tttatttatt tctttgctcg |
| 7561 | ttctgctcat tccttcactc ctccagtgga catgtgttgt tcaatgcccc gtgctaggcc |
| 7621 | tcagcatgca cagacatgtt ggggaccagc ctcaacgcca cccgtagggt tcctgaagtc |
| 7681 | cattggtgac acaggaatga gaagagacag gttaagagtt cataaagagt gggggccagg |
| 7741 | gggccaattg caaaatggag gctgcaaaag gctcagagct ctggtctcca cactattttt |
| 7801 | tgagtacagt cactcagatc taagaagcag atgttcaggg agaaacagtg aaagggaggc |
| 7861 | agtgggtcat aggcgtaatc tatagcaata gagttttaaa tgaatctcct ttgtgctcaa |
| 7921 | acagcatgtc tttaaattat cggagagtag ctggtggaag tgggcttagc tagaagactg |
| 7981 | catgtctgtc caatgcttca aaggagggtc tttctccttg aacagagtgt ttacagataa |
| 8041 | gacagggggt ctcactctga gcatgggaac atgatggcaa ttaggaggct tttcttctca |
| 8101 | gaggcctctt gtggctttcc acaacttatt gtctcatatt tttatggaca gtttatacag |
| 8161 | gcaccccaca agtccttttc ccaacatgcc cccctccctt tttttttttt taaccgctat |
| 8221 | tgctattatg gcttatttgt ggtgtttggt ctgttttcag aagtgtcttt tgcatctgta |
| 8281 | gactaaaagt aaacagcata aacagataca cattaaagta aaatttgtaa tagttgatcc |
| 8341 | tttaatggtc ttaatctgtt taagaggatt tatgtttgaa agtccgtcag tagctccaat |
| 8401 | gagaatgtca gtctcaggca ggagggttaa atgagcctga gatgctttaa aaacctgttt |
| 8461 | ttttaaaatt tggttatatt taatgttaaa tttttatttt tttcttttag atgatgtcta |
| 8521 | actttttaaa aatgatgttt agtagtatta tacgaatggg gagttatgta gaaattggaa |
| 8581 | gtatttcaat tacattgtac ttctaattga tgttttaagt ttattgtacg atcttccatt |
| 8641 | taaataacag tctgtctaag atcatttgtt tgatttgtca attgttggtc tatttgggtc |
| 8701 | tgagaattcc acaattttga ggaatttttt gttaactatt tatatatttt gtagtttgaa |
| 8761 | cagaggagtg taaagcaatt ccagcagccg cagcagtagc tgtgactgca ataaggccca |
| 8821 | taagactgtt ataagggtaa aaataaatct ctttgttttg gtaaacactt ttttttaaaa |
| 8881 | catttttgtg acaatatgaa tggaaggaga ggctttctaa ggtctattga gggaaaccag |
| 8941 | tatccaaact cctttcttag tttttatcag taacacagat gtttttacac cgaacgtgga |
| 9001 | attaatacag gtgaaaaggt gacagttttg acaagtaata gtttgagaat taggtcgaat |
| 9061 | gtcaatattt ttgaccatta acataaaagg agggttgaca caactctgaa tgggcactgt |
| 9121 | tttgttggaa gaaaactgat acgcaaattg aagtttttaa cctttttttt ttaaagataa |
| 9181 | tatatttttt tctaaactta aatatgagat tgggccatta ttaactttca taatttggag |
| 9241 | tgtttagggc ctattattgg attaattatt ttgggatgtg ggccagctgt actaaaattg |
| 9301 | gtccaaatta tgggaaaatg agcacgtttt tcagtgtaag tagtgttacc tttttgatag |
| 9361 | tatagtttct gttttagttt tgtcttgtat ttattatttt gatgggtaca attaactgta |
| 9421 | aaggtcccct caggggacca attaatgaca atttcatagg aattattttg tagtaccata |
| 9481 | gtgtgatcag agatgtaatt ttttttaatt aatattttta aattatttga ccattgttaa |
| 9541 | ggttgttggc acctcttttt tgggggctta aactgttaat tgaattgaac tctgtgaatg |
| 9601 | atccgggctc catccagaaa ataaatgata ggatactggt ctttgattat gacctggaat |
| 9661 | tttaactagt caatgttgtc ggtagccttt taggcaaccg atagttggcc ttatgtaaag |
| 9721 | aggggggaac tgataaccta tggacacatt tattaacttt tttttttttc ctttgggtga |
| 9781 | gagggcccat gagtatttgt aggcttaggg atccaaacgc tattattaac ataaacttca |
| 9841 | actgggggtt ttaaccatgt gacaggccta attaaaggca ggaatgggac acatgcccaa |
| 9901 | taggtataat tttgggctgt tgtagccaca ggtttgttag gcgaggaggt cactgttttt |
| 9961 | attttggctt tgtattctag gattagtaaa taacagaaga caaacatgag tataattagt |
| 10021 | aacttttttt tttagtaaaa gagtgacctg tagtgttact tggcatctta gtttactata |
| 10081 | tgttattaat gaggaacccc actgggggta tgttaattta ttctagctaa gcagttatgt |
| 10141 | tattagaagc tgagaagggg gtgtttgtta aagtaacagg gcagaagaaa ggcggattta |
| 10201 | agatacgagc ttaatacagt gtagcaggta taggtagtag gcaaagtgag agaattaaaa |
| 10261 | atgaataaat tatttggctt agacttttgt ttttttagta taatgtctga ggcctgtgtt |
| 10321 | gtttgtggaa gtcgcattgt tgaggctgta gttcctgtag ggtctttttt aggctggttc |
| 10381 | aaatgttttt ttatttttta attttttatc ctttgatgag gatgtagtct ttaggctggt |
| 10441 | actggaaatt ttaggagtgg cgtctgtgtt aagagacttt ttacaatttt taaagagcag |
| 10501 | gttagtgttt taagaaaaac ttgtgtttta ttttaatgtt tagtttatag aaaactggat |
| 10561 | gatatctttt taactttagt aaatacgttt acacacggaa ttttttacaa ttatcatttt |
| 10621 | aaaacttgtt tagatcttta aaacaaaatt aaacaacctt ttttgtataa attttttata |
| 10681 | acttttttta tgacttttac agacaatttt taacatgtct taacttttta tgttttataa |
| 10741 | tttttttact aaaggtacat ttttataact ttttaaattt ttttactttt ttgtattttt |
| 10801 | ttgatttttg tcttagtctt ttttttactt ttattttttt aaatgtgtaa taattagatg |
| 10861 | agtgttggta acaatggatg tatgtacata ttttagtttt taaaatttag ggatgtgttt |
| 10921 | aacatctgtt tgccagaact gactaggttc caattcttta cggttaacac ctattgaagg |
| 10981 | agggtatgtg cctgtgagct ggtaatctgg gcattgtggg ataatttgtt tagccagcct |
| 11041 | ctgtgtaagt tgaaattatt tagataagtt tctccaattt tggtggaata atcgatgtga |
| 11101 | ttgggtggct tggtcaagca gtgatgtcat aacctgaagg tctgcttgat tattgccgta |
| 11161 | agccaatggg ccaggcagag agctgtgggc tcgaatgtgt gtaataaaag taggatgtgt |
| 11221 | accttggtct agtaattgtt gaagttgaag aaaaagacca cacagagtgg gctccagagc |
| 11281 | aaacttaagg ctgtaatagt ttttaaataa atacacagaa taaccttagc tctctgaatg |
| 11341 | ttagtaaatt cagatcaagt gattggatta tgtggtctcc accagactgt tgctttttca |
| 11401 | tgtttaccag acccaccagt aaaaacagct atggctcctt ccaaaggggc atcacaagta |
| 11461 | atttttggaa gaacctatgt agttaatttt aagaattgaa aagtttttag gataatgatt |
| 11521 | attaatacat ccaacaaatt ttgttaaatt aatctgtcat gtaactgagt taataaatgc |
| 11581 | ctgtttaacc tgatttttat ttattggaac tataattttt attgggctca gtgccacaaa |
| 11641 | atttaataat tcatatatga gcctgtccaa ttagaattgc catctgattt aagtatactg |
| 11701 | taagtgcttt tatggtatta tgtggcaaaa aggaccattt aactaaatca tcattttgaa |
| 11761 | caataacccc cattattgtg tggttagtgt gaagtaggga acacaatgaa ttataaaggc |
| 11821 | aagtctgagt caatcctact gacctgggct tgctgaattt tgttttcaat tactgataac |
| 11881 | tctttcatgg cctcgggtgt tagttctctg ttactgcgta agttggtatt tcccctcaat |
| 11941 | attgagaaga gattagacat agcataagta ggaattgcta aattgggcca aatccaatta |
| 12001 | atatcttcta acaatttttg aaaattattt aaggttttga aagaatctct tctaatttga |
| 12061 | accttttgag gcttaatggc tctatcctgt acttgtattt tcaaatactg aaaaggagtg |
| 12121 | gttgtttgaa ttttgtcagg tgctataagt aattcagcat ttgtaattgt cttttgcaaa |
| 12181 | gattaataat attgaataag ttggtctcta ctttttgctg cacaaatctg gaaactgatc |
| 12241 | tctaacaggc tggatagttc tgcctacaaa agtttgacaa actgtgggac tatttaacat |
| 12301 | accctggggc aaaactttcc aatgatattt ggctgcaggt tttttgttat taacggcagg |
| 12361 | aatggtaaag gcaaattttt tgaaatctgc ctctgctaaa ggaattgtaa aaaagcagtc |
| 12421 | ttttaaatct ataataacaa gcggtcagtc tttagggagc acagtggggg atgggagccc |
| 12481 | aggttgtaag gctcccatcg gttgaattac agcgttgacg ccatctaccg gactttttct |
| 12541 | taattacaaa tactggggaa ttccaaggag agaaagtggg tgaaatatat ccttttttta |
| 12601 | gtagtttatt ttataaagca cccccaactt ttccttaggg agcggccact gttcaaccca |
| 12661 | gacggggcgc cgggtcatcc attttaaggg aaattgctcc ttcactgtaa taactgtagg |
| 12721 | gtgaacctga attgccccat ctccataatg aactgtgggt cgggcaataa tgggcacggt |
| 12781 | gagccaagtc tcgggctccc tccccctgca cccactcggc tgaggaggag gtggccattc |
| 12841 | tggacatttc tctacaggaa ccgtgggctg aacaattttt tgagtaggtt tagggagact |
| 12901 | ggggagattg gcataaatca tcttcagact ctcctttttg ttagtactcg gtagaggtgg |
| 12961 | ttcagagttc tgattatcaa actcctctct ctcctcctct gactcagcct cattatctgt |
| 13021 | ctgaaaaggc tccagtgctg catgcaccaa tgaccaaagc gaccaaacag gcaaaggaat |
| 13081 | ttcctttcct tctctatatg ctcttttaag gtcctttcca actccttctt aatgttttaa |
| 13141 | tttcaaagtt tcctgttttg ggaaccaagg gcaaaattgt tccatagcat gaaacaaatc |
| 13201 | cataagattt tccgtatcaa cttttacccc accatgcatg cttgaagagc tgccgtagga |
| 13261 | agctcaaata cgtggtgtac ttactttcag tttttcccat tgtgtcccta gctttctctg |
| 13321 | ggcgccccgc ttacctgtag aggttaaaac ttttatgtcc ttgggagtcc tttgttcgtt |
| 13381 | ggtcctctgt ttcacatgct tgagcgtttc ctcaccagat tcttttgggc cccacgttgg |
| 13441 | gcgccagaat gttggggacc agcctcaaca ccacctgtag ggtacctgaa gtctggtggt |
| 13501 | gacaaaggaa tgagaagaga caggttaaga gttcataaag agtggaggcc agggggccaa |
| 13561 | ttgcaaaatg gaggctgcaa aaggctcaga gctctggtct ccacactatt tattgagtac |
| 13621 | aataacttag atctaagaag cagatgttca gggcaaaaca gtgaaagggt agcagtgcgt |
| 13681 | cacaggcata atctacagca gaagcgcttt aaatgaatct cctttgtgct caaacagcat |
| 13741 | atctttaact tatcggagag tagctagtgg gagtgggctt aactaggagc ctgcacgtct |
| 13801 | gtccacattc caatgcttca aaggagggtc tttctccttg aatacagtgt ttacagataa |
| 13861 | gagagagcag gtctcgctct gagcatggca attaggaggc ttttctcctc agaggcctct |
| 13921 | tgtggctttc cacaacttat tgtcccatat ttttatggcc agtttataca ggcaccccac |
| 13981 | aagtcctttt cccaacacag acaggaatac ggcagcctgt gccctgggag ctcactgtct |
| 14041 | tgtgggaggg aaccactcaa gccactcccc acttgtcctc ctgtccctct cttcttgggc |
| 14101 | tctgtccccc acctctctct gtcctttgtc ttgcaggtgg ggagatggag gaggcagagc |
| 14161 | tcacatcctg gtattttgtg tcatctccct tctccttgga tcttagcaag accaagcgac |
| 14221 | accttgtgcc tggggccccc ttcctgctgc aggtttcttc cagaggggaa ggatgagtag |
| 14281 | ggaggatgtg gtagttagga gggctcaggg tctgaccact ctcttttgcc tgccctcctt |
| 14341 | tacctgccta ggccttggtc cgtgagatgt caggctcccc agcttctggc attcctgtca |
| 14401 | aagtttctgc cacggtgtct tctcctgggt ctgttcctga agtccaggac attcagcaaa |
| 14461 | acacagacgg gagcggccaa gtcagcattc caataattat ccctcagacc atctcagagc |
| 14521 | tgcagctctc agtaggactc ctcggacccc tgggagatgg tgggggaagg ggaggagggt |
| 14581 | gagctggggt cccaaggatc catggcctga cttgggggga aggtggggta cttggctctg |
| 14641 | agctactacc ctattcgcac ctgaccccct ctccaggtat ctgcaggctc cccacatcca |
| 14701 | gcgatagcca ggctcactgt ggcagcccca ccttcaggag gccccgggtt tctgtctatt |
| 14761 | gagcggccgg attctcgacc tcctcgtgtt ggggacactc tgaacctgaa cttgcgagcc |
| 14821 | gtgggcagtg gggccacctt ttctcattac tactacatgg tgtgcatgag ctggggagtc |
| 14881 | acggagggct ggggtgcagg gaagagccct ctgggtgggg ctgggggggt tcaaggctga |
| 14941 | ggctgtccca tgaagaggca accactcttg tccctcccat tcttggccca gatcctatcc |
| 15001 | cgagggcaga tcgtgttcat gaatcgagag cccaagagga ccctgacctc ggtctcggtg |
| 15061 | tttgtggacc atcacctggc accctccttc tactttgtgg ccttctacta ccatggagac |
| 15121 | cacccagtgg ccaactccct gcgagtggat gtccaggctg gggcctgcga gggcaaggtg |
| 15181 | accggggtca ggagagatgg cacttgtgcc gagggggttg aggacagggt gattgccaac |
| 15241 | agggcatgga tttagcttgg gggcagtgag gataccggga ctgaaggaag ctctcccact |
| 15301 | ctgaccgccc ccacctgccg cccctgccag ctggagctca gcgtggacgg tgccaagcag |
| 15361 | taccggaacg gggagtccgt gaagctccac ttagaaaccg actccctagc cctggtggcg |
| 15421 | ctgggagcct tggacacagc tctgtatgct gcaggcagca agtcccacaa gcccctcaac |
| 15481 | atgggcaagg tttgtccaga ccctctccac agctctctca cccctccatg gctcatcccc |
| 15541 | ctgcttccct gagccttggg cgcagcccct ggatcccact gaggctcccc acagtctctt |
| 15601 | ccccacttgg ccctgtggtc tccatctcct ggctctgtat cctttcctat ccccccatgt |
| 15661 | gctgccctct cacctgtgcc gagtgctcag tcctgcccct cagccacact tggctcctag |
| 15721 | cattcctgcc tttcttgcag gtctttgaag ctatgaacag ctatgacctc ggctgtggtc |
| 15781 | ctgggggtgg ggacagtgcc cttcaggtgt tccaggcagc gggcctggcc ttttctgatg |
| 15841 | gagaccagtg gaccttatcc agaaagagtg agaacagaga aggaagggga gtgggtggcg |
| 15901 | ggaagataag gaaggaggaa gggcctgagg ggaccagctg gaagagtccg ggcaggaagg |
| 15961 | gctgggcagg ggaaggggag gaggggagga ggccgagtgc ctgacggctg gactgcagcc |
| 16021 | tttctctcta ccaggactaa gctgtcccaa ggagaagaca acccggaaaa agagaaacgt |
| 16081 | gaacttccaa aaggcgatta atgagaaatg tgagttgcgg gtgcctaggc agtagcttgg |
| 16141 | gctctccacc tgggatccgg gttgggggtc tgcctctctg cccctcggct ccttgctgaa |
| 16201 | cccacgtgtg gtatttgggg ccagagatcc gaattccggg attacgagtg gaaggtgggc |
| 16261 | agctctctcc agcagcctct cttatgttgc tggtctcaag gggtcggggc gggggctgag |
| 16321 | gtgtatgtcc tttttgtcct ctcatgctca cccccacctg gccctgcagt gggtcagtat |
| 16381 | gcttccccga cagccaagcg ctgctgccag gatggggtga cacgtctgcc catgatgcgt |
| 16441 | tcctgcgagc agcgggcagc ccgcgtgcag cagccggact gccgggagcc cttcctgtcc |
| 16501 | tgctgccaat ttgctgagag tctgcgcaag aagagcaggg acaagggcca ggcgggcctc |
| 16561 | caacgaggtg aggggctggg tggggctagg gcacaggtgg cggcgcttgg aaaggcagaa |
| 16621 | cggtcccctc ctcactcccg tccaccgtgg tcccccagcc ctggagatcc tgcaggagga |
| 16681 | ggacctgatt gatgaggatg acattcccgt gcgcagcttc ttcccagaga actggctctg |
| 16741 | gagagtggaa acagtggacc gctttcaaat gtgagagtgt gtgccggccc ggccttttct |
| 16801 | ctgtgctgtg tctcggggcc agccggggta gacgggcctt ctctgccttt ccctacacag |
| 16861 | attgacactg tggctccccg actctctgac cacgtgggag atccatggcc tgagcctgtc |
| 16921 | caaaaccaaa ggtgatgtca ccctgtctgg gcctcaggtg accctgcttc catttccctg |
| 16981 | taccccagct ccctgttccc tttgctctta gtgtaggaag agggtccagt gatctgggga |
| 17041 | ggtctgtgcc agcgtgcagc tggcgtgggc cagagggcag aggcggactg agacagagct |
| 17101 | gggtcacccc cacccctccc tcctgtggcc ctgaagcttt gatggcccct ctgatctctg |
| 17161 | cccctgtgcc cacgcttcct ttccctcagg cctatgtgtg gccaccccag tccagctccg |
| 17221 | ggtgttccgc gagttccacc tgcacctccg cctgcccatg tctgtccgcc gctttgagca |
| 17281 | gctggagctg cggcctgtcc tctataacta cctggataaa aacctgactg tgaggcccca |
| 17341 | tgggagcctg agcatacagg agttggggga gccagggccc agtgaggggt ggggaggcta |
| 17401 | accgggccag gactctggcc atcctcgttt tcctgccctc aggtgagcgt ccacgtgtcc |
| 17461 | ccagtggagg ggctgtgcct ggctgggggc ggagggctgg cccagcaggt gctggtgcct |
| 17521 | gcgggctctg cccggcctgt tgccttctct gtggtgccca cggcagccac cgctgtgtct |
| 17581 | ctgaaggtgg tggctcgagg gtccttcgaa ttccctgtgg gagatgcggt gtccaaggtt |
| 17641 | ctgcagattg aggtgaatgg agcacccctg aatataagtc cccgggcccc cagctttgtc |
| 17701 | ctccaccctc agcactctct ctgctggcca ggccaggggc ccaacaccca aaccaatgcc |
| 17761 | ttggtctgtt cccatcttct acaattctga tccaactctg tccctggagt tgaaactcaa |
| 17821 | agttctgggg gagtctgcgc tagcagggca ggctgtagtc ctgtgtgacc tcacaaccat |
| 17881 | gttttccctg agacagaagg aaggggccat ccatagagag gagctggtct atgaactcaa |
| 17941 | ccccttgggt gagtgaccct ctacctccag ccattggttt cctaagtggg tacaggtggt |
| 18001 | gggggatgtg gacagcagga caggctgcca acttccccca tttccccaga ccaccgaggc |
| 18061 | cggaccttgg aaatacctgg caactctgat cccaatatga tccctgatgg ggactttaac |
| 18121 | agctacgtca gggttacagg tgggagtgcc ctttagtccc ttcccagtgg ccaccttcgg |
| 18181 | attcatgtgg gacttgtgga tccctgcttg gtcccactcc ccgtgagcct ctgacacaga |
| 18241 | gtcctcagac ctccaccctc tccctcccat gtagcctcag atccattgga cactttaggc |
| 18301 | tctgaggggg ccttgtcacc aggaggcgtg gcctccctct tgaggcttcc tcgaggctgt |
| 18361 | ggggagcaaa ccatgatcta cttggctccg acactggctg cttcccgcta cctggacaag |
| 18421 | acagagcagt ggagcacact gcctcccgag accaaggacc acgccgtgga tctgatccag |
| 18481 | aaaggttctg ggtgcaaggg caagcaggag gggggccagg aaaggacagt tactggaaga |
| 18541 | tggacagccc aggaggctac agagggaaag aaagggggcc cctgatgagg atggggagca |
| 18601 | tggccttggg ctcaaacagc agaagggtga gtgtcacctg agcggccacc tctcctctcc |
| 18661 | aaggctacat gcggatccag cagtttcgga aggcggatgg ttcctatgcg gcttggttgt |
| 18721 | cacggggcag cagcacctgg tgagcttggg agagtggttc cagggttctg agggggtcag |
| 18781 | ggctggggca ggggtgggac agagctggta tgatgggagg gtggataacc aggcacctgg |
| 18841 | gggcgtgggc ataatgagaa gcaagtcctt atccccaacc ctcctttcct gccctccagg |
| 18901 | ctcacagcct ttgtgttgaa ggtcctgagt ttggcccagg agcaggtagg aggctcgcct |
| 18961 | gagaaactgc aggagacatc taactggctt ctgtcccagc agcaggctga cggctcgttc |
| 19021 | caggacctct ctccagtgat acataggagc atgcaggtgc gggcatgctg gggctggccc |
| 19081 | gagaagcgcc tgtcggagga ctctctttgc cccttccccc tcctgtttga catcttttct |
| 19141 | ccccttacta ggggggtttg gtgggcaatg atgagactgt ggcactcaca gcctttgtga |
| 19201 | ccatcgccct tcatcatggg ctggccgtct tccaggatga gggtgcagag ccattgaagc |
| 19261 | agagagtggt aagttcagtg gcgtttctgc cctctgctgg cccccagctc tctccctttt |
| 19321 | tcctcaggaa cccaggggtc caggcccaag accctcctcc cgttttcttc caggaagcct |
| 19381 | ccatctcaaa ggcaagctca tttttggggg agaaagcaag tgctgggctc ctgggtgccc |
| 19441 | acgcagctgc catcacggcc tatgccctga cactgaccaa ggcccctgcg gacctgcggg |
| 19501 | gtgttgccca caacaacctc atggcaatgg cccaggagac tggaggtgag gggtgagggg |
| 19561 | ctctggcagt gagcctgagg cccaggggac cttaggatcc ctgagtgtgc ccagagggag |
| 19621 | aggctggatg aagactcaga ggaggaatga agttataagc aggggtgggt tgggggagac |
| 19681 | tcaggagagc ccagcagggg gtggctaagg gccaggggac caggctcttc tccctgcctt |
| 19741 | cctgtttact cgtggtctcc cttcactttc agataacctg tactggggct cagtcactgg |
| 19801 | ttctcagagc aatgccgtgt cgcccacccc ggctcctcgc aacccatccg accccatgcc |
| 19861 | ccaggcccca gccctgtgga ttgaaaccac agcctacgcc ctgctgcacc tcctgcttca |
| 19921 | cgagggcaaa gcagagatgg cagaccaggc tgcggcctgg ctcacccgtc agggcagctt |
| 19981 | ccaaggggga ttccgcagta cccaagtagg ggccgtcccc gggctctggc gggggtgggt |
| 20041 | agtcctcaga ccaagggctt gcttgagtcc tggctcaacc tccctaggac acggtgattg |
| 20101 | ccctggatgc cctgtctgcc tactggattg cctcccacac cactgaggag aggggtctca |
| 20161 | atgtgactct cagctccaca ggccggaatg ggttcaagtc ccacgcgctg cagctgaaca |
| 20221 | accgccagat tcgcggcctg gaggaggagc tgcaggtgaa ccactccctg gtgaaccact |
| 20281 | ccctcgcctg ggtagccagg acacctgggc ctcgtggcca ggccagaagc cgtccccacc |
| 20341 | ctcccacccg tggaatcccc gcagcacttc ttcctggggt cttcggggga agactgactt |
| 20401 | cctggctgcg tgacctggag ctctgagctt cagttttctc acttgtagag taacatacac |
| 20461 | agagttcacc ctacagggtc gttagaaggc tgaagtgaga taattcatgt gctggtataa |
| 20521 | actttgtgga aatgtgaggt ggggagaggg ggtggggctg ttttgaggaa ggagataagt |
| 20581 | tattggagcc gcaaaaacag gtttgcttgt gcccttctaa catcgccttc ccttttctgt |
| 20641 | tgctgaagtt ttccttgggc agcaagatca atgtgaaggt gggaggaaac agcaaaggaa |
| 20701 | ccctgaaggt gagggccagg gaaggggtgg ggccaggcac tggtggagga gagggtgtgg |
| 20761 | agtgagaggc ctgtgggcag aggcacatgg tccggggaag gaggcagaca cctcagggtt |
| 20821 | ggtgtcccgt gcttccgtcc tgggtgtttt tccccctgct tgctttcgct tgctctcccc |
| 20881 | atctctgggt acctgttgtt tcctttaccc gcctcagtgc tggtggctcc gaatcccact |
| 20941 | cctcagccca ggcctcttcc ctgaaccatg ggccccactc gtcccactcc cacagcacct |
| 21001 | cagacgaggc atgtcccaaa gcccttcttc attctgtgtc tcttgtctgg ctggtgggag |
| 21061 | cccctcccag ccaggagccc agccactact ctagaggccg tgttagtggc ccctctccca |
| 21121 | agcctgtcct tatgtcccta gtgactcctc ctctgctccc ctgctgcctg tggcccttgg |
| 21181 | tgctgcatcc tagattctgt gctgagacgg ccttctccct acctggaact tctctctacc |
| 21241 | tcctgtctcc cctgtctgat ccactgtcca cacggcagtg acactgacct tccaaaagcc |
| 21301 | ccagccagat cagccttggg gaaaagtcac tccccgctgc ccacggctca gatggctggg |
| 21361 | cctctgccca cccctccggc cagacagctc tccttgtcta cacagatccc cttgcctttc |
| 21421 | ctgtccttcc ctgcttcttg gcccacagga caagctcttt cttctccttc aagccttggc |
| 21481 | cagaagcctt tcctgagctt ttcagtccag cctcttccca gcacagtctg gagtgttggc |
| 21541 | ctctgggggc aggcccctgc ttctttacct ctctgtctcg cctgacgcct gtggcgaatg |
| 21601 | tggtgccact cgtgtgtgtg gactgtgcag tgacggggag gaaaaggggc tgaaggcctc |
| 21661 | aaatcctgta gcccagggag atgcccttag gtatggcacc agagaggtct gtggcctcac |
| 21721 | atgtcccacg tcctctccct gccccttgct gagccaggtc cttcgtacct acaatgtcct |
| 21781 | ggacatgaag aacacgacct gccaggacct acagatagaa gtgacagtca aaggccacgt |
| 21841 | cgagtacacg agtgagtgtg ggggttggga ggccttgggg ccaggcaggg gctggcgcag |
| 21901 | ggagccgggt ggccatccca gccctcctca caatgcttcc ctgtgcagtg gaagcaaacg |
| 21961 | aggactatga ggactatgag tacgatgagc ttccagccaa ggatgaccca gatgcccctc |
| 22021 | tgcagcccgt gacacccctg cagctgtttg agggtcggag gaaccgccgc aggagggagg |
| 22081 | cgcccaaggt ggtggaggag caggagtcca gggtgcacta caccgtgtgc atctggtggg |
| 22141 | cgccgggagc tgccctgggc caggggaggg agggcaggac ccaggctggg gctgggcttc |
| 22201 | tggagcccgc gcaggcagaa cctggacgac agctcacacg tctccacagg cggaacggca |
| 22261 | aggtggggct gtctggcatg gccatcgcgg acgtcaccct cctgagtgga ttccacgccc |
| 22321 | tgcgtgctga cctggagaag gtgtggtcag ccacccaggg caaccccctc tgtcccaggt |
| 22381 | actgagccct gtcatgtgca gggcctgtga ccaactcccc ttttccacag ctgacctccc |
| 22441 | tctctgaccg ttacgtgagt cactttgaga ccgaggggcc ccacgtcctg ctgtattttg |
| 22501 | actcggtgag tggggagaga tgaggcagga agggactcga tggcaccggg tttactgagt |
| 22561 | atgcgttagg aggtttctca ggagacagct gtgtcagcgg ctggtgctct tgagaacttg |
| 22621 | tgatgtcatc agagagaagg acaagaatgt gagcccgtga gacacagcag agtaaggggc |
| 22681 | agacctgcag gcggcaggga ccgatgccag tcagcaggga ccctcagggt ttgagaggga |
| 22741 | gtctttccta atgctggttt tattcagctt gaggggctgc ctttgttttt ttgttgaact |
| 22801 | tcctatcttt tttttaatat taaagcgtat tttcctttac aaagtgatgg tggccataga |
| 22861 | tgatagttgt atttgtcttt tcacgacctt atttggctaa aatagttatc aaccctctta |
| 22921 | cggctctcaa aacattttta tttatttatt tagtaaagac agggtctcgc tctgttgccc |
| 22981 | aggctggtct tgaactcccg gcctcaagcg atcctctggc ctaggccttt caaagtaccg |
| 23041 | gatttacagg ccagagccac catgcccggc cttcaaaaaa agttttggaa catttactgt |
| 23101 | aacctctggg agaaaatgtg agaaaggtgt ggtggctgtc attagccagc tgtttgtagg |
| 23161 | tcagggagac ccctacccag tgtgtgcaga ggggccagcc cccatcagct ggggaagcct |
| 23221 | ggctgacaca tctgggttga acacaataga aaacacagag ccaacaagat tcccggatag |
| 23281 | ggagctgacg gtgcagcagc ctagctcagg agggacactg gcacggcacc gtgtggactg |
| 23341 | ggcccgcgtg ggcacgagga ggggtcaggc ctgggacctg agtcgggggg tcaggcagga |
| 23401 | tgacagaacc tgcagttagg ttgtggcaaa taaaggagga cccagttgta tccatgacaa |
| 23461 | agatgaggcc gcgaggaggg cgagtgggtt tgggggcagg cagagtgcct tggagaactt |
| 23521 | acaggtcctg ccacaatcct aatgcaagga tggagctgca agttcagttt gggaatcatc |
| 23581 | agcctggatt ggtttggtgg aagccaggga gtggttgaga cccccacagg ggagctctga |
| 23641 | ggaaggaagt tccgaaggag ggaacgtaag aaatgaccag gtcagaacca agggtggtcc |
| 23701 | agaagctaac ccttagctta gggacagttt cacagagaac acgtccatga tgcaagactc |
| 23761 | tgctgagggc ctggagcagt gaagactggg gcaaggtcac cctctgggaa gtgaagtcac |
| 23821 | cagagacctt gcggagcagc tttgagagtt ctctgagtag gaaggtaaca gaatgtgaag |
| 23881 | gacactggag agaaggccaa taggaagcaa acaaaaacag gccaaggaaa cccagtacag |
| 23941 | ggggctgcag ggcccaggga gtgggtccct catctctcct ccccacgctt ggccaggtcc |
| 24001 | ccacctcccg ggagtgcgtg ggctttgagg ctgtgcagga agtgccggtg gggctggtgc |
| 24061 | agccggccag cgcaaccctg tacgactact acaaccccgg tgagcactgc aggacaccct |
| 24121 | gaaattcagg agaactttgg cataggtgcc ctcctatggg acaatggaca ccggggtagt |
| 24181 | gagggggcag agagccctgg ggctccctgg gactgaggag gcagaatgga ggggcctgtg |
| 24241 | ccctaactcc tctctgttct ccagagcgca gatgttctgt gttttacggg gcaccaagta |
| 24301 | agagcagact cttggccacc ttgtgttctg ctgaagtctg ccagtgtgct gagggtgaga |
| 24361 | ctgagggcct ggggcggggc agtggaggcg ggatggccgg ggcccccccc acactgtctg |
| 24421 | atgggttccc caacttcagg gaagtgccct cgccagcgtc gcgccctgga gcggggtctg |
| 24481 | caggacgagg atggctacag gatgaagttt gcctgctact acccccgtgt ggagtacggt |
| 24541 | cagtcttccc accgaggccc tggcctgacc ctccctcggg gaccggccgt tttggtctct |
| 24601 | ctgggtgtag cctgctcctc ttacaggtca tgcacgcagc ctgtttgctc tgacaccaac |
| 24661 | ttcctaccct ctcagcctca aagtaactca cctttccccc ttctcctcac cccctcttag |
| 24721 | gcttccaggt taaggttctc cgagaagaca gcagagctgc tttccgcctc tttgagacca |
| 24781 | agatcaccca agtcctgcac ttcagtatga agcaaaccgg agaggcgggc agggctgggg |
| 24841 | ggagacaggg aggctgaggt gtggccgagg acctgaccat ctggaagtgt gaaaatcccc |
| 24901 | ttgggctgtc agaagccttg ggcttggcca taaataggga ggcagtggca cctctccatg |
| 24961 | ggggtggcga aggtggaatg agaggatcta cacagagtcc ccagcctggg ctcaccctgc |
| 25021 | accttctctt cccctctgac cacttttgcg cacgtcatcc ccgcagccaa ggatgtcaag |
| 25081 | gccgctgcta atcagatgcg caacttcctg gttcgagcct cctgccgcct tcgcttggaa |
| 25141 | cctgggaaag aatatttgat catgggtctg gatggggcca cctatgacct cgagggacag |
| 25201 | tgagtcatct ggtcccctca gtctcttgtc ctccccatgc ctcgccacct aggccttgcc |
| 25261 | cctcagaagc cagatgcctg tgctctccgt ttccacctgc catcctcccg agccctgctg |
| 25321 | actgcccctt tgccccctgc agcccccagt acctgctgga ctcgaatagc tggatcgagg |
| 25381 | agatgccctc tgaacgcctg tgccggagca cccgccagcg ggcagcctgt gcccagctca |
| 25441 | acgacttcct ccaggagtat ggcactcagg ggtgccaggt gtgagggctg ccctcccacc |
| 25501 | tccgctggga ggaacctgaa cctgggaacc atgaagctgg aagcactgct gtgtccgctt |
| 25561 | tcatgaacac agcctgggac cagggcatat taaaggcttt tggcagcaaa gtgtcagtgt |
| 25621 | tggcagtgaa gtgtcagtgt gtgttgctag ggctgagagc agtgcccctg cccgatgcag |
| 25681 | ttctgggcag gccaggttga cataacctta gactctctga gccctgatga cccttgggct |
| 25741 | gttcagctct gctagaacct cccagatgac ccgctaggag tctagtgctt cacaggacca |
| 25801 | ccccgagcag aactgggacc caagagcctg caccccaagg accagagtcc atgccaagac |
| 25861 | cacccttcag cttccaaggc cctccactgc ccggctgtcg ccagtcacca cggcctcaga |
| 25921 | cagggcttgt gctcagctga cacctgtgac acagctcttc tgcctcatga gctgttgtcc |
| 25981 | agctacacct ccccgactct gtcctcgtgc tgctggcggt tctgaggtct gcagatttta |
| 26041 | gctgagttcc gggctgttga aagcctgctg acgcttggtt ctgttatcag tggaatgagg |
| 26101 | tgactttccc ggagttgtgc aatcctcagg tccggcagtg tcttcttcca gttactggtt |
| 26161 | tcaaacaagc caaaagtctg actttggtgt gtttgtgaat cctctgagga agccgctgtt |
| 26221 | ctcctggggt ctccccttcc caccggacct gcctaacttt cccccattta gtggcacacc |
| 26281 | tggggtcttc agagatgact ccgcgtctgt ccaaagaagt ttggtgagat cagtttccgt |
| 26341 | agaggtcatg acagttcagc agcctgccat ccagtcattc gacagaaatt cgggaatctt |
| 26401 | tcacttcatg ccatgccctg tgccaggtgc cagagataca gctgctcact ccagggctca |
| 26461 | tcgctgggga gacagataag aggacgggca gtccccaccc tctgtgaaag atgtgatgtc |
| 26521 | agggagcagt gtggtcctgt ggggcatcta accaagtcag gggcattgcc aggcagggac |
| 26581 | agggaaggct tcctggagca ggtggcctcc aagtggggct ctgaagactg agaaggagcc |
| 26641 | aggaaaagag caggggtaga tgagggcatc tggggcagaa ggagaatata caaaggccca |
| 26701 | gaggccgggg gcaggacagg gtacctttgg ggacattgca tgtaattgac cacattcgga |
| 26761 | gtttggattt ggaagtggtg gaagagatgg agatggtgag acaagtagta agcacgtcag |
| 26821 | ccttccaggt gcgctccttt ccgatgagca ctgtcttatc ccacgtaact ttgagaagtt |
| 26881 | tgggcctttc ccactgtggc agaggtttcc tgaggctctt gcatacatgg ccctatggtt |
| 26941 | gctcatcaga tctttctccc agtagctgct cagcatggtg gtggcataag cccattttcc |
| 27001 | ggagccaggg attcagttgc agcaagacat ggcccggtct gggaggtcaa ccatgaagaa |
| 27061 | ggcagtagct gtcattgccc aaccccagaa atcccaatcc tgttttctcc ctctcagtcc |
| 27121 | tgatcatgga ttcagcagca gcgaactcgc caatgtagtg ggtggcacag ccagggtctt |
| 27181 | gactctggct ctgcagtagc acagtctgga aaagctctga ggggagagag acccccactg |
| 27241 | gtccgagggt ctggcacaga gccagaaatg ggggggaagg tatggggctg ggtcgcctct |
| 27301 | gacctctcag gtaccatcca ggaggccctg gcctctcact gaacccggcc actcctcttt |
| 27361 | ggcatggcct cttcccaaat ccccaaactg cctccttacc cacaaaagtg gtctctgagt |
| 27421 | gtcagtccag tgggaccccc accccttatg gcttcagttc cccaaatagg gctggaccct |
| 27481 | tgatcctgat ccagctgtgg ctatccagcc ccttcctggg gactttggac tttgaggggg |
| 27541 | gcatgcccag ttgtgctggg aatccatact ttccctggct ggagtagaac ctgtggactg |
| 27601 | tagtcctgag ggcagtcatg ttct |

By “effective amount” is meant the amount of a required to ameliorate the symptoms of a disease relative to an untreated patient. In particular embodiments, the disease is an autoimmune disorder of a corona virus disorder (Covid-19). In some embodiments, an effective amount is determined by the patient's gender, where a male subject received more of a C4 inhibitor than a female subject. In other embodiments, a female subject receives an increased amount of a C4 agonist relative to a male subject. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence driven by its promoter.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, a “human endogenous retrovirus” or “HERV” polynucleotide sequence is a polynucleotide sequence that occurs in the human genome that is substantially identical to a sequence in a retrovirus or that was derived from a retrovirus. In some embodiments, the HERV sequence is a human endogenous retrovirus type K (HERV-K) sequence. In some other embodiments, the HERV sequence is a C4-HERV sequence. In certain embodiments, a retroviral (C4-HERV) sequence in intron 9 is inserted within a C4A polynucleotide sequence or a C4B polynucleotide sequence. An exemplary HERV sequence is provided at GenBank Accession No. AF164613.1, and is reproduced below.

| | |
|---|---|
| 1 | tgtggggaaa agcaagagag atcaaattgt tactgtgtct gtgtagaaag aagtagacat |
| 61 | aggagactcc attttgttat gtgctaagaa aaattcttct gccttgagat tctgttaatc |
| 121 | tatgacctta cccccaaccc cgtgctctct gaaacgtgtg ctgtgtcaac tcagggttga |
| 181 | atggattaag ggcggtgcag gatgtgcttt gttaaacaga tgcttgaagg cagcatgctc |
| 241 | cttaagagtc atcaccactc cctaatctca agtacccagg gacacaaaaa ctgcggaagg |
| 301 | ccgcagggac ctctgcctag gaaagccagg tattgtccaa ggtttctccc catgtgatag |
| 361 | tctgaaatat ggcctcgtgg gaagggaaag acctgaccgt cccccagccc gacacctgta |
| 421 | aagggtctgt gctgaggagg attagtaaaa gaggaaggaa tgcctcttgc agttgagaca |
| 481 | agaggaaggc atctgtctcc tgcctgtccc tgggcaatgg aatgtctcgg tataaaaccc |
| 541 | gattgtatgc tccatctact gagataggga aaaaccgcct tagggctgga ggtgggacct |
| 601 | gcgggcagca atactgcttt gtaaagcatt gagatgttta tgtgtatgca tatccaaaag |
| 661 | cacagcactt aatcctttac attgtctatg atgccaagac ctttgttcac gtgtttgtct |
| 721 | gctgaccctc tccccacaat tgtcttgtga ccctgacaca tccccctctt tgagaaacac |
| 781 | ccacagatga tcaataaata ctaagggaac tcagaggctg gcgggatcct ccatatgctg |
| 841 | aacgctggtt ccccgggtcc ccttatttct ttctctatac tttgtctctg tgtctttttc |
| 901 | ttttccaaat ctctcgtccc accttacgag aaacacccac aggtgtgtag gggcaaccca |
| 961 | cccctacatc tggtgcccaa cgtggaggct tttctctagg gtgaaggtac gctcgagcgt |
| 1021 | ggtcattgag gacaagtcga cgagagatcc cgagtacatc tacagtcagc cttacggtaa |
| 1081 | gcttgcgcgc tcggaagaag ctagggtgat aatggggcaa actaaaagta aaattaaaag |
| 1141 | taaatatgcc tcttatctca gctttattaa aattctttta aaaagagggg gagttaaagt |
| 1201 | atctacaaaa aatctaatca agctatttca aataatagaa caattttgcc catggtttcc |
| 1261 | agaacaagga acttcagatc taaaagattg gaaaagaatt ggtaaggaac taaaacaagc |
| 1321 | aggtaggaag ggtaatatca ttccacttac agtatggaat gattgggcca ttattaaagc |
| 1381 | agctttagaa ccatttcaaa cagaagaaga tagcatttca gtttctgatg cccctggaag |
| 1441 | ctgtttaata gattgtaatg aaaacacaag gaaaaaatcc cagaaagaaa ccgaaagttt |
| 1501 | acattgcgaa tatgtagcag agccggtaat ggctcagtca acgcaaaatg ttgactataa |
| 1561 | tcaattacag gaggtgatat atcctgaaac gttaaaatta gaaggaaaag gtccagaatt |
| 1621 | aatggggcca tcagagtcta aaccacgagg cacaagtcct cttccagcag gtcaggtgct |
| 1681 | cgtaagatta caacctcaaa agcaggttaa agaaaataag acccaaccgc aagtagccta |
| 1741 | tcaatactgg ccgctggctg aacttcagta tcggccaccc ccagaaagtc agtatggata |
| 1801 | tccaggaatg cccccagcac cacagggcag ggcgccatac catcagccgc ccactaggag |
| 1861 | acttaatcct atggcaccac ctagtagaca gggtagtgaa ttacatgaaa ttattgataa |
| 1921 | atcaagaaag gaaggagata ctgaggcatg gcaattccca gtaacgttag aaccgatgcc |
| 1981 | acctggagaa ggagcccaag agggagagcc tcccacagtt gaggccagat acaagtcttt |
| 2041 | ttcgataaaa atgctaaaag atatgaaaga gggagtaaaa cagtatggac ccaactcccc |
| 2101 | ttatatgagg acattattag attccattgc ttatggacat agactcattc cttatgattg |
| 2161 | ggagattctg gcaaaatcgt ctctctcacc ctctcaattt ttacaattta agacttggtg |
| 2221 | gattgatggg gtacaagaac aggtccgaag aaatagggct gccaatcctc cagttaacat |
| 2281 | agatgcagat caactattag gaataggtca aaattggagt actattagtc aacaagcatt |
| 2341 | aatgcaaaat gaggccattg agcaagttag agctatctgc cttagagcct gggaaaaaat |
| 2401 | ccaagaccca ggaagtacct gcccctcatt taatacagta agacaaggtt caaaagagcc |
| 2461 | ctaccctgat tttgtggcaa ggctccaaga tgttgctcaa aagtcaattg ccgatgaaaa |
| 2521 | agccggtaag gtcatagtgg agttgatggc atatgaaaac gccaatcctg agtgtcaatc |
| 2581 | agccattaag ccattaaaag gaaaggttcc tgcaggatca gatgtaatct cagaatatgt |
| 2641 | aaaagcctgt gatggaatcg gaggagctat gcataaagct atgcttatgg ctcaagcaat |
| 2701 | aacaggagtt gttttaggag gacaagttag aacatttgga ggaaaatgtt ataattgtgg |
| 2761 | tcaaattggt cacttaaaaa agaattgccc agtcttaaac aaacagaata taactattca |
| 2821 | agcaactaca acaggtagag agccacctga cttatgtcca agatgtaaaa aaggaaaaca |
| 2881 | ttgggctagt caatgtcgtt ctaaatttga taaaaatggg caaccattgt cgggaaacga |
| 2941 | gcaaaggggc cagcctcagg ccccacaaca aactggggca ttcccaattc agccatttgt |
| 3001 | tcctcagggt tttcagggac aacaaccccc actgtcccaa gtgtttcagg gaataagcca |
| 3061 | gttaccacaa tacaacaatt gtccctcacc acaagcggca gtgcagcagt agatttatgt |
| 3121 | actatacaag cagtctctct gcttccaggg gagcccccac aaaaaatccc tacaggggta |
| 3181 | tatggcccac tgcctgaggg gactgtagga ctaatcttgg gaagatcaag tctaaatcta |
| 3241 | aaaggagttc aaattcatac tagtgtggtt gattcagact ataaaggcga aattcaattg |
| 3301 | gttattagct cttcaattcc ttggagtgcc agtccaagag acaggattgc tcaattatta |
| 3361 | ctcctgccat atattaaggg tggaaatagt gaaataaaaa gaataggagg gcttgtaagc |
| 3421 | actgatccaa caggaaaggc tgcatattgg gcaagtcagg tctcagagaa cagacctgtg |
| 3481 | tgtaaggcca ttattcaagg aaaacagttt gaagggttgg tagacactgg agcagatgtc |
| 3541 | tctattattg ctttaaatca gtggccaaaa aactggccta aacaaaaggc tgttacagga |
| 3601 | cttgtcggca taggcacagc ctcagaagtg tatcaaagta tggagatttt acattgctta |
| 3661 | gggccagata atcaagaaag tactgttcag ccaatgatta cttcaattcc tcttaatctg |
| 3721 | tggggtcgag atttattaca acaatggggt gcggaaatca ccatgcccgc tccattatat |
| 3781 | agccccacga gtcaaaaaat catgaccaag atgggatata taccaggaaa gggactaggg |
| 3841 | aaaaatgaag atggcattaa agttccagtt gaggctaaaa taaatcaaga aagagaagga |
| 3901 | atagggtatc ctttttaggg gcggtcactg tagagcctcc taaacccata ccactaactt |
| 3961 | ggaaaacaga aaaaccggtg tgggtaaatc agtggccgct accaaaacaa aaactggagg |
| 4021 | ctttacattt attagcaaat gaacagttag aaaagggtca cattgagcct tcgttctcac |
| 4081 | cttggaattc tcctgtgttt gtaattcaga agaaatcagg caaatggcat acgttaactg |
| 4141 | acttaagggc tgtaaacgcc gtaattcaac ccatggggcc tctccaaccc gggttgccct |
| 4201 | ctccggccat gatcccaaaa gattggcctt taattataat tgatctaaag gattgctttt |
| 4261 | ttaccatccc tctggcagag caggattgtg aaaaatttgc ctttactata ccagccataa |
| 4321 | ataataaaga accagccacc aggtttcagt ggaaagtgtt acctcaggga atgcttaata |
| 4381 | gtccaactat ttgtcagact tttgtaggtc gagctcttca accagtgaga gaaaagtttt |
| 4441 | cagactgtta tattattcat tatattgatg atattttatg tgctgcagaa acgaaagata |
| 4501 | aattaattga ctgttataca tttctgcaag cagaggttgc caatgctgga ctggcaatag |
| 4561 | catctgataa gatccaaacc tctactcctt ttcattattt agggatgcag atagaaaata |
| 4621 | gaaaaattaa gccacaaaaa atagaaataa gaaaagacac attaaaaaca ctaaatgatt |
| 4681 | ttcaaaaatt actaggagat attaattgga ttcggccaac tctaggcatt cctacttatg |
| 4741 | ccatgtcaaa tttgttctct atcttaagag gagactcaga cttaaatagt caaagaatat |
| 4801 | taaccccaga ggcaacaaaa gaaattaaat tagtggaaga aaaaattcag tcagcgcaaa |
| 4861 | taaatagaat agatccctta gccccactcc aacttttgat ttttgccact gcacattctc |
| 4921 | caacaggcat cattattcaa aatactgatc ttgtggagtg gtcattcctt cctcacagta |
| 4981 | cagttaagac ttttacattg tacttggatc aaatagctac attaatcggt cagacaagat |
| 5041 | tacgaataac aaaattatgt ggaaatgacc cagacaaaat agttgtccct ttaaccaagg |
| 5101 | aacaagttag acaagccttt atcaattctg gtgcatggca gattggtctt gctaattttg |
| 5161 | tgggacttat tgataatcat tacccaaaaa caaagatctt ccagttctta aaattgacta |
| 5221 | cttggattct acctaaaatt accagacgtg aacctttaga aaatgctcta acagtattta |
| 5281 | ctgatggttc cagcaatgga aaagcagctt acacagggcc gaaagaacga gtaatcaaaa |
| 5341 | ctccatatca atcggctcaa agagcagagt tggttgcagt cattacagtg ttacaagatt |
| 5401 | ttgaccaacc tatcaatatt atatcagatt ctgcatatgt agtacaggct acaagggatg |
| 5461 | ttgagacagc tctaattaaa tatagcatgg atgatcagtt aaaccagcta ttcaatttat |
| 5521 | tacaacaaac tgtaagaaaa agaaatttcc cattttatat tactcatatt cgagcacaca |
| 5581 | ctaatttacc agggcctttg actaaagcaa atgaacaagc tgacttactg gtatcatctg |
| 5641 | cactcataaa agcacaagaa cttcatgctt tgactcatgt aaatgcagca ggattaaaaa |
| 5701 | acaaatttga tgtcacatgg aaacaggcaa aagatattgt acaacattgc acccagtgtc |
| 5761 | aagtcttaca cctgcccact caagaggcag gagttaatcc cagaggtctg tgtcctaatg |
| 5821 | cattatggca aatggatgtc acgcatgtac cttcatttgg aagattatca tatgttcatg |
| 5881 | taacagttga tacttattct tattcacatt tcatatgggc aacttgccaa acaggagaaa |
| 5941 | gtacttccca tgttaaaaaa catttattgt cttgttttgc tgtaatggga gttccagaaa |
| 6001 | aaatcaaaac tgacaatgga ccaggatatt gtagtaaagc tttccaaaaa ttcttaagtc |
| 6061 | agtggaaaat ttcacataca acaggaattc cttataattc ccaaggacag gccatagttg |
| 6121 | aaagaactaa tagaacactc aaaactcaat tagttaaaca aaaagaaggg ggagacagta |
| 6181 | aggagtgtac cactcctcag atgcaactta atctagcact ctatacttta aattttttaa |
| 6241 | acatttatag aaatcagact actacttctg cagaacaaca tcttactggt aaaaagaaca |
| 6301 | gcccacatga aggaaaacta atttggtgga aagataataa aaataagaca tgggaaatag |
| 6361 | ggaaggtgat aacgtgaggg agaggttttg cttgtgtttc accaggagaa aatcagcttc |
| 6421 | ctgtttggtt acccactaga catttgaagt tctacaatga acccatcgga gatgcaaaga |
| 6481 | aaagggcctc cacggagagg gtaacaccag tcacatggat ggataatcct atagaagtat |
| 6541 | atgttaatga tagtgtatgg gtacctggcc ccatagatga tcgctgccct gccaaacctg |
| 6601 | aggaagaagg gatgatgata aatatttcca ttgggtatcg ttatcctcct atttgcctag |
| 6661 | ggagagcacc aggatgttta atgcctgcag tccaaaattg gttggtagaa gtacctactg |
| 6721 | tcagtcccat cagtagattc acttatcaca tggtaagcgg gatgtcactc aggccacggg |
| 6781 | taaattattt acaagacttt tcttatcaaa gatcattaaa atttagacct aaagggaaac |
| 6841 | cttgccccaa ggaaattccc aaagaatcaa aaaatacaga agttttagtt tgggaagaat |
| 6901 | gtgtggccaa tagtgcggtg atattataaa acaatgaatt tggaactatt atagattggg |
| 6961 | cacctcgagg tcaattctac cacaattgct caggacaaac tcagtcgtgt ccaagtgcac |
| 7021 | aagtgagtcc agctgttgat agcgacttaa cagaaagttt agacaaacat aagcataaaa |
| 7081 | aattgcagtc tttctaccct tgggaatggg gagaaaaagg aatctctacc ccaagaccaa |
| 7141 | aaatagtaag tcctgtttct ggtcctgaac atccagaatt atggaggctt actgtggcct |
| 7201 | cacaccacat tagaatttgg tctggaaatc aaactttaga aacaagagat tgtaagccat |
| 7261 | tttatactgt cgacctaaat tccagtctaa cagttccttt acaaagttgc gtaaagcccc |
| 7321 | cttatatgct agttgtagga aatatagtta ttaaaccaga ctcccagact ataacctgtg |
| 7381 | aaaattgtag attgcttact tgcattgatt caacttttaa ttggcaacac cgtattctgc |
| 7441 | tggtgagagc aagagagggc gtgtggatcc ctgtgtccat ggaccgaccg tgggaggcct |
| 7501 | caccatccgt ccatattttg actgaagtat taaaaggtgt tttaaataga tccaaaagat |
| 7561 | tcatttttac tttaattgca gtgattatgg gattaattgc agtcacagct acggctgctg |
| 7621 | tagcaggagt tacattgcac tcttctgttc agtcagta |

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation can be at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level, copy number, sequence, or activity that is associated with a disease or disorder or risk of disease or disorder.

As used herein, “obtaining” as in “obtaining an agent” includes synthesizing, purchasing, or otherwise acquiring the agent.

As used herein a “probe” or “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition. In some embodiments, a “reference copy number” is a copy number of 0 or 1. In some other embodiments, a “reference level” is a level of C4A or C4B polynucleotide, such as C4A or C4B RNA, or a C4 (e.g., C4A or C4B) polypeptide in a healthy, normal subject, or in a subject that does not have a disease or altered levels of the polynucleotide or protein in question. In some embodiments, the amount of C4A or C4B in a male subject is compared to the amount in a female subject.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, or at least about 25 amino acids. The length of the reference polypeptide sequence can be about 35 amino acids, about 50 amino acids, or about 100 amino acids. For nucleic acids, the length of the reference nucleic acid sequence will generally be at least about 50 nucleotides, at least about 60 nucleotides, or at least about 75 nucleotides. The length of the reference nucleic acid sequence can be about 100 nucleotides, about 300 nucleotides or any integer thereabout or therebetween.

In some embodiments, the reference sequence is a sequence of a “short form” of complement component 4A (C4A) genomic polynucleotide. In some other embodiments, the reference sequence is the sequence of a short form of complement component 4B (C4B) genomic polynucleotide. As used herein, a “short form” of a C4A or C4B polynucleotide is a C4A or C4B polynucleotide that does not contain an insertion of a human endogenous retrovirus (HERV) sequence. As used herein, a “long form” of a C4A or C4B polynucleotide is a C4A or C4B polynucleotide that contains an insertion of a human endogenous retrovirus (HERV) sequence.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity. In some embodiments, an siRNA or other inhibitory nucleic acid targets C4a expression.

By “specifically binds” is meant an agent that recognizes and binds a polypeptide or polynucleotide of the invention, but which does not substantially recognize and bind other molecules in a sample, for example, a biological sample, which naturally includes a polynucleotide of the invention. In some embodiments, the agent is a nucleic acid molecule. In some embodiments, the agent is an antibody that specifically binds C4A polypeptide.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., and at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 .mu.g/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., and at least about 68° C. In one embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, “autoimmune disease treatment” or “treatment for Covid-19” includes, without limitation, agents that modulate C4 expression or activity.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B present depictions of the analysis of C4 gene variation by whole-genome sequencing. FIG. 1A shows distributions (across 1,265 individuals) of total C4 gene copy number (C4A+C4B), as measured from read depth of coverage across the C4 locus, in whole-genome sequencing data. FIG. 1B shows the relative numbers of reads that overlap sequences specific to C4A or C4B (together with the total C4 gene copy number, FIG. 1A) are used to infer the underlying copy numbers of the C4A and C4B genes. For example, in an individual with four C4 genes, the presence of equal numbers of reads specific to C4A or C4B suggests the presence of two copies each of C4A and C4B. Precise statistical approaches (including inference of probabilistic dosages), and further approaches for phasing C4 allelic states with nearby SNPs to create reference haplotypes, are described below.

FIGS. 2A-2E present graphs and plots showing the association of SLE with C4 alleles. FIG. 2A illustrates the levels of SLE risk associated with 11 common combinations of C4A and C4B gene copy number. Each circle reflects the level of SLE risk (odds ratio) associated with a specific combination of C4A and C4B gene copy numbers relative to the most common combination (two copies of C4A and two copies of C4B) in shades of gray. The area of each circle is proportional to the number of individuals with that number of C4A and C4B genes. Paths from left to right on the plot reflect the effect of increasing C4A gene copy number; paths from bottom to top reflect the effect of increasing C4B gene copy number; and diagonal paths from upper left to lower right reflect the effect of exchanging C4B for C4A copies. Data are from analysis of 6,748 SLE cases and 11,516 controls of European ancestry. The odds ratios are reported with confidence intervals in FIG. 7. FIG. 2B illustrates the association of SLE with genetic markers (SNPs and imputed HLA alleles) across the extended MHC locus within the European-ancestry cohort. Orange diamond: an initial estimate of C4-related genetic risk, calculated as a weighted sum of the number of C4A and C4B gene copies: (2.3)C4A+C4B, with the weights derived from the relative coefficients estimated from logistic regression of SLE risk vs. C4A and C4B gene dosages. This risk score is imputed with an accuracy (r2) of 0.77. Points representing all other genetic variants in the MHC locus are shaded according to their level of linkage disequilibrium-based correlation to this C4-derived risk score. FIG. 2C illustrates the SLE risk associated with common combinations of C4 structural allele and MHC SNP haplotype. For each C4 locus structure, separate odds ratios are reported for each “haplogroup,” 160 i.e., the MHC SNP haplotype background on which the C4 structure segregates. Error bars represent 95% confidence intervals around the effect size estimate for each sex. FIG. 2D reflects what is shown in the graph in FIG. 2B, except with a cohort of 673 Sjögren's Syndrome (SjS) cases and 1,153 controls of European ancestry. The gray diamond is also an estimate of C4-related genetic risk calculated as a weighted 165 sum of C4A and C4B gene copies estimated from a logistic regression of SjS risk: (2.3)C4A+C4B. FIG. 2E reflects what is shown in the graph in FIG. 2C, except with the SjS cohort from FIG. 2D. Error bars represent 95% confidence intervals around the effect size estimate for each sex.

FIGS. 3A-3D present plots showing a C4 and trans-ancestral analysis of the MHC association signal in SLE. FIG. 3A shows that common C4 alleles exhibit similar strengths of association (odds ratios) in European ancestry and African American (1,494 SLE cases; 5,908 controls) cohorts. Error bars represent 95% confidence intervals around the effect size estimate for each sex. FIG. 3B depicts an analysis of SLE risk across combinations of C4-B(S) and DRB1*03:01 genotypes in an African American SLE case-control cohort, in which the two alleles exhibit very little LD (r2=0.10). On each DRB1*03:01 genotype background, additional C4-B(S) alleles increase risk (ie. within each grouping). Whereas on each C4-B(S) background, DRB1*03:01 alleles have no appreciable relationship with risk (ie. every nth point from each group). Error bars represent 95% confidence intervals around the effect size estimate for each combination of C4-B(S) and DRB1*03:01. FIG. 3C depicts a trans-ancestry comparison of the association of genetic markers with SLE (unconditioned log-odds ratios) among European-ancestry (x-axis) and African American (y-axis) research participants. LD with C4-derived risk in European-ancestry individuals (indicated by gray shading) contributes to the apparent discordance of association patterns between populations. A lead SNP identified below, rs2105898, is among the strongest signals in the African American cohort; among Europeans, though, its association is initially much less remarkable than that of other SNPs that are in strong LD with C4. FIG. 3D depicts the results of an analyses controlling for C4-derived risk, analyses of European ancestry and African American cohorts both identified a small haplotype (tagged by rs2105898) harboring a genetic signal independent of C4. Several SNPs that form a short haplotype common to both ancestry groups are among the top associations in both cohorts. Further analyses of this haplotype are described in Example 3 herein. Many SNP associations that appear specific to the European-ancestry cohort have European-ancestry LD with rs2105898 in excess of LD with the same haplotype in the African American cohort (FIGS. 12A and 12B).

FIGS. 4A-4I present plots and graphs showing sex differences in the magnitude of C4 genetic effects and complement protein concentrations. FIG. 4A shows SLE risk (odds ratios) associated with the four most common C4 alleles in men (x-axis) and women (y-axis) among 6,748 affected and 11,516 unaffected individuals of European ancestry. For each sex, the lowest-risk allele (C4-A(L)-A(L)) is used as a reference (odds ratio of 1.0). Shading of each allele reflects the relative level of SLE risk conferred by C4A and C4B copy numbers as in FIG. 2C. Error bars represent 95% confidence intervals around the effect size estimate for each sex. FIG. 4B shows schizophrenia risk (odds ratios) associated with the four most common C4 305 alleles in men (x-axis) and women (y-axis) among 28,799 affected and 35,986 unaffected individuals of European ancestry, aggregated by the Psychiatric Genomics Consortium. For each sex, the lowest-risk allele (C4-B(S)) is used as a reference (odds ratio of 1.0). For visual comparison with FIG. 4A, shading of each allele reflects the relative level of SLE risk. Error bars represent 95% confidence intervals around the effect size estimate for each sex. FIG. 4C shows the relationship between male bias in SLE risk (difference between male and female log-odds ratios) and LD with C4 risk for common (minor allele frequency [MAF]>0.1) genetic markers across the extended MHC region. For each SNP, the allele for which sex risk bias is plotted is the allele that is positively correlated (via LD) with C4-derived risk score. FIG. 4D shows the relationship between male bias in SjS risk (log-odds ratios) and LD with C4 risk for common (minor allele frequency [MAF]>0.1) genetic markers across the extended MHC region. For each SNP, the allele for which sex risk bias is plotted is the allele that is positively correlated (via LD) with C4-derived risk score. FIG. 4E shows the relationship of male bias in schizophrenia risk (log-odds ratios) and LD to C4A expression for common (MAF>0.1) genetic markers across the extended MHC region. For each SNP, the allele for which sex risk bias is plotted is the allele that is positively correlated (via LD) with imputed C4A expression. FIG. 4F shows the concentrations of C4 protein in cerebrospinal fluid sampled from 340 adult men (blue) and 167 adult women (pink) as a function of age with local polynomial regression (LOESS) smoothing. Concentrations are normalized to the number of C4 gene copies in an individual's genome (a strong independent source of variance, FIG. 11A) and shown on a log₁₀ scale. Shaded regions represent 95% confidence intervals derived during LOESS smoothing. FIG. 4G shows the levels of C3 protein in cerebrospinal fluid from 179 adult men and 125 adult women as a function of age. Concentrations are shown on a log₁₀ scale. Shaded regions represent 95% confidence intervals derived during LOESS smoothing. FIG. 4H shows the levels of C4 protein in blood plasma from 182 adult men and 1662 adult women as a function of age. As in FIG. 4F, concentrations are normalized to C4 gene copy number (FIG. 11B) and shown on a log₁₀ scale. Shaded regions represent 95% confidence intervals derived during LOESS smoothing. FIG. 4I shows the levels of C3 protein in blood plasma as a function of age from the same individuals in FIG. 4H. Concentrations are shown on a log₁₀ scale. Shaded regions represent 95% confidence intervals derived during LOESS smoothing.

FIG. 5 presents a panel of 2,530 reference haplotypes (created from whole-genome sequence (WGS) data) containing C4 alleles and SNPs across the MHC locus that enables imputation of C4 alleles into large-scale SNP data. The SNP haplotypes flanking each C4 allele are shown as rows, with white and black representing the major and minor allele of each SNP as columns, respectively. Gray lines at the bottom indicate the physical location of each SNP along chromosome 6. The differences among the haplotypes are most pronounced closest to C4 (toward the center of the plot), as historical recombination events in the flanking megabases will have caused the haplotypes to be less consistently distinct at greater genomic distances from C4. The patterns indicate that many combinations of C4A and C4B gene copy numbers have arisen recurrently on more than one SNP haplotype, a relationship that can be used in association analyses (FIG. 2C).

FIGS. 6A and 6B present a tablular depiction and plots showing the aggregation of joint C4A and C4B genotype probabilities per individual across imputed C4 structural alleles for estimation of SLE risk for each combination. FIG. 6A illustrates that an individual's joint C4A and C4B gene copy number can be calculated by summing the C4A and C4B gene contents for each possible pair of two inherited alleles. Many pairings of possible inherited alleles result in the same joint C4A and C4B gene copy number. FIG. 6B shows the results after each individual's C4A and C4B gene copy number was imputed from their SNP data, using the reference haplotypes summarized in FIG. 5. For >95% of individuals (exemplified by samples 1-6 in the figure), this inference can be made with >90% certainty/confidence (the areas of the circles represent the posterior probability distribution over possible C4A/C4B gene copy numbers). For the remaining individuals (exemplified by samples 7-9 in the figure), greater statistical uncertainty persists about C4 genotype. To account for this uncertainty, in downstream association analysis, all C4 genotype assignments are handled as probabilistic gene dosages—analogous to the genotype dosages that are routinely used in large-scale genetic association studies that use imputation.

FIG. 7 presents dot plots of SLE odds ratios and confidence intervals for each combination of C4A and C4B gene copy number. Odds ratios and 95% confidence intervals underlying each of the C4-genotype risk estimates in FIG. 2A are presented as a series of panels for each observed copy number of C4B, with increasing copy number of C4A for that C4B dosage (x-axis).

FIGS. 8A-8C present plots showing the relationship between the association with SLE and linkage to C4 for variants in the MHC region. FIG. 8A illustrates the relationship between SLE association [−log 10(p), y-axis] and LD to the weighted C4 risk score (x-axis) for genetic markers and imputed HLA alleles across the extended MHC locus. In this European ancestry cohort, it is unclear (from this analysis alone) whether the association with the markers in the predominant ray of points (at a ˜45° angle from the x-axis) is driven by variation at C4 or by the long haplotype containing DRB1*03:01, DQA1*05:01, and B*08:01. In addition, at least one independent association signal (a ray of points at a higher angle in the plot, with strong association signals and only weak LD-based correlation to C4 and DRB1*0301) with some LD to DRB1*15:01 is also present. FIG. 8B is as in FIG. 8A but among the European-ancestry SjS cohort. Similar to SLE, it is unclear whether the effect is driven by variation at C4 or linked HLA alleles, DRB1*03:01, DQA1*05:01, and B*08:01. There is also an independent association signal with LD to DRB1*15:01. FIG. 8C shows an analysis of an African American SLE case-control cohort, in which LD in the MHC region is more limited, identified a set of markers that associate with SLE in proportion to their correlation with the C4 composite risk score inferred from the earlier analysis of the European cohort, which itself associates with SLE at p<10-18. No similar relationship is observed for DRB1*03:01 and other alleles linked in European ancestry haplotypes. An independent association signal is also present in this cohort, more clearly in LD with the DRB1*15:03 allele.

FIGS. 9A and 9B present graph plots presenting conditional association analyses for genetic markers across the extended MHC locus within the European-ancestry cohort. FIG. 9A shows an association of SLE with genetic markers (SNPs and imputed HLA alleles) across the extended MHC locus within the European-ancestry cohort controlling for C4 composite risk (weighted sum of risk associated with various combinations of C4A and C4B). Variants are shaded by their LD with rs2105898, an independent association identified from trans-ancestral analyses. FIG. 9B is as in FIG. 9A, but in association with a European-ancestry SjS cohort. Here a simpler linear model of risk contributed by C4A and C4B was used instead of a weighted sum across all possible combinations.

FIGS. 10A-10D present plots and graphs showing the correlation of C4 protein measurements (in cerebrospinal fluid (CSF) and blood plasma) with imputed C4 gene copy number. FIG. 10A shows measurements of C4 protein in CSF obtained by ELISA, which are presented as log₁₀(ng/mL) (y-axis) for each observed or imputed copy number of total C4 (x-axis, here showing most likely copy number from imputation). Because C4 gene copy number affects C4 protein levels so strongly, C4 protein measurements were normalized by C4 gene copy number in subsequent analyses (FIG. 4F). FIG. 10B shows measurements of C4 protein in blood plasma obtained by immunoturbidimetric assays, which are presented as log₁₀(mg/dL) (y-axis) for each best-guess imputed copy number of total C4 (x-axis). Because C4 gene copy number affects C4 protein levels so strongly, C4 protein measurements were normalized by C4 gene copy number in subsequent analyses (FIG. 4H). Due to the number of observations (n=1,844 total), downsampling arrived at the 500 points shown, but median and quartiles shown are for all individuals per C4 copy number. FIG. 10C shows the results of C4 protein measured in blood plasma in 670 individuals with SjS (gray) and 1,151 individuals without SjS (black) as shown on a log₁₀ scale (x-axis). Vertical stripes represent median levels for cases and controls separately. FIG. 10D is as in FIG. 10C, but concentrations are normalized to the number of C4 gene copies in an individual's genome and this per-copy amount is shown on a log₁₀ scale (x-axis).

FIGS. 11A-11G present graphs showing that the concordance of trans-ancestral SLE risk association patterns across the MHC region is largely a function of strong European LD between C4 and nearby variants. FIG. 11A illustrates LD in European ancestry between the composite C4 risk term (weighted sum of risk associated with various combinations of C4A and C4B) and variants in the MHC region as r2 (y-axis). FIG. 11B is as in FIG. 11A, but for African Americans. FIG. 11C illustrates LD for the same variants measured in European ancestry individuals (x-axis) and African Americans (y-axis). Note the abundance of variants that have greater LD with C4 across European ancestry individuals, with several groups of variants that have similar LD in European ancestry individuals but which exhibit a range of LD in African Americans. FIG. 11D illustrates associations with SLE for the same variants in European ancestry cases and controls (x-axis) and African American cases and controls (y-axis). Variants are shaded by their LD with C4 in patterns of trans-ancestral associations with SLE risk in the MHC region. FIG. 11E is as in FIG. 11D, but controls for the effect of C4 in only European ancestry associations (x-axis). Note that this greatly aligns the patterns of association across the MHC region between European ancestry and African American cohorts. FIG. 11F is as in FIG. 11E, but controls for the effect of C4 in African American associations as well (y-axis). Note that this does not significantly affect the concordance seen in FIG. 11E due to the lack of broad LD relationships between C4 and variants in the MHC region in African Americans. The independent signal, rs2105898, and HLA alleles, DRB1*15:01 and DRB1*15:03, are also highlighted. FIG. 11G is as in FIG. 11F, but with variants noted by whether they exhibit greater LD to rs2105898 in European ancestry individuals or African Americans. Note that the independent DRB1*15:01/DRB1*15:03 association may be largely due to LD with rs2105898, with the relative strength of association for each in a particular cohort due to ancestry-specific LD with the haplotype defined by rs2105898. (DRB1*15:03 is largely an African-restricted allele, and DRB1*15:01 may be picking up signal in African Americans during imputation—beyond the small fraction of admixed haplotypes—due to small dosages assigned by the classifier in haplotypes that likely have DRB1*15:03.)

FIGS. 12A and 12B present a pictorial gene expression map and a ZNF143 consensus sequence motif related to the effect of rs2105898 alleles on concordance with known ZNF143 binding motif in XL9 region. FIG. 12A shows the location of rs2105898 (line at center) within the XL9 region, with relevant tracks showing overlapping histone marks and transcription factor binding peaks (from ENCODE50), visualized with the UCSC genome browser. FIG. 12B shows a ZNF143 consensus binding motif as a sequence logo, with the letters showing if the base is present in >5% of observed instances. The alleles of rs2105898 are indicated by an outlined box surrounding the base.

FIG. 13 presents a tabular depiction of the imputation accuracy for C4 copy numbers in European ancestry and African American haplotypes. Accuracy was determined by cross-validation of the reference panel with directly-typed C4 copy numbers from WGS data. Aggregated copy numbers imputed from each round of leaving 10 samples out were then correlated with the directly-typed measurements and reported as r2 for each type of copy number variation for European ancestry and African American members of the reference panel separately.

FIG. 14 presents a tabular depiction of the frequency of common C4 alleles and their linkage with HLA alleles in European ancestry and African American cohorts. For each common C4 allele and HLA gene, the allele with highest LD (r2) is listed if present on more than half of the haplotypes with that C4 allele (exact fraction in %). r2 values higher than 0.4 are bolded to point out particularly strong C4-HLA allele pairings, such as for several with the C4-B(S) allele in European ancestry individuals. Some common C4 alleles are further subdivided into distinct haplotypes used in imputation (and in FIG. 2C), as defined by shared alleles from variants flanking C4. Note that some alleles, such as C4-A(L)-A(L)-3, are present at a frequency in African Americans that may solely reflect their presence on a fraction (˜15-20/o) of admixed haplotypes spanning this region, whereas others, such as C4-B(S), are likely to also exist on African haplotypes—these differences between C4 alleles are also reflected in the similarity of LD with HLA alleles to the corresponding row of the European ancestry section.

FIG. 15 presents a tabular depiction of logistic regression models of SLE risk against C4 variation, HLA alleles, and/or rs2105898 in European ancestry and African American cohorts. Coefficients (beta, standard error) and p-values (as −log₁₀(p)) for individual terms composing several relevant logistic regression models for predicting SLE risk that also include ancestry-specific covariates. For each model, the Akaike information criterion (AIC) and overall p-value (as determined by Chi-squared likelihood-ratio test) are given at the right end to indicate the relative strengths between similar models for each ancestry cohort.

### DETAILED DESCRIPTION OF THE INVENTION

The invention features compositions and methods that are useful for the treatment of autoimmune disorders.

The invention is based, at least in part, on the discovery that the complement component 4 (C4) genes in the MHC locus, recently found to increase risk for schizophrenia, generate 7-fold variation in risk for lupus (95% CI: 5.88-8.61; p<10-117 in total) and 16-fold variation in risk for Sjögren's syndrome (95% CI: 8.59-30.89; p<10-23 in total), with C4A protecting more strongly than C4B in both illnesses. The same alleles that increase risk for schizophrenia, greatly reduced risk for lupus and Sjögren's syndrome. In all three illnesses, C4 alleles acted more strongly in men than in women: common combinations of C4A and C4B generated 14-fold variation in risk for lupus and 31-fold variation in risk for Sjögren's syndrome in men (vs. 6-fold and 15-fold among women respectively) and affected schizophrenia risk about twice as strongly in men as in women. At a protein level, both C4 and its effector (C3) were present at greater levels in men than women in cerebrospinal fluid (p<10-5 for both C4 and C3) and plasma among adults ages 20-50, corresponding to the ages of differential disease vulnerability. Sex differences in complement protein levels may help explain the larger effects of C4 alleles in men, women's greater risk of SLE and Sjögren's, and men's greater vulnerability in schizophrenia. These results nominate the complement system as a source of sexual dimorphism in vulnerability to diverse illnesses.

### Complement Component 4 (C4A And C4B) Genes

The complement component 4 (C4A and C4B) genes are present in the MHC locus, between the class I and class II HLA genes. Classical complement proteins help eliminate debris from dead and damaged cells, attenuating the exposure of diverse intracellular proteins to the adaptive immune system. C4A and C4B commonly vary in genomic copy number and encode complement proteins with distinct affinities for molecular targets. SLE frequently presents with hypocomplementemia that worsens during flares, possibly reflecting increased active consumption of complement. Rare cases of severe, early-onset SLE can involve complete deficiency of a complement component (C4, C2, or C1Q) and one of the strongest common-variant associations in SLE maps to ITGAM, which encodes a receptor for C3, the downstream effector of C4. Though total C4 gene copy number associates with SLE risk, this association is thought to arise from linkage disequilibrium (LD) with nearby HLA alleles, which have been the focus of fine-mapping analyses.

### Reporting the Status

Additional embodiments of the invention relate to the communication of assay results, characterization of disease, or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis is communicated to the subject as soon as possible after the diagnosis is obtained. The diagnosis may be communicated to the subject by the subject's treating physician. Alternatively, the diagnosis may be sent to a subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Hardware and Software

The methods described herein, analyses can be performed on general-purpose or specially-programmed hardware or software. One can then record the results (e.g., characterization of autoimmune disease (e.g., SLE, SjS) on tangible medium, for example, in computer-readable format such as a memory drive or disk or simply printed on paper. The results also could be reported on a computer screen.

In aspects, the analysis is performed by a software classification algorithm. The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the data to indicate the number of target sequences detected (e.g., by using a biochip containing targeted baits), and optionally the strength of a signal. Data analysis can include steps of determining signal strength and removing data deviating from a predetermined statistical distribution. For example, observed peaks can be normalized, by calculating the height of each peak relative to some reference. The reference can be background noise generated by the instrument and chemicals such as the energy absorbing molecule which is set at zero in the scale.

In aspects, software used to analyze the data can include code that applies an algorithm to the analysis of the results. The software also can also use input data (e.g., sequence data or biochip data) to characterize autoimmune disease (e.g., SLE, SjS).

### Methods of Treatment of Autoimmune and Inflammatory Disorders

The present invention provides methods of treating autoimmune and/or inflammatory disorders, or symptoms thereof which comprise administering a therapeutically effective amount of a pharmaceutical composition comprising an agent that modulates C4 expression or activity to a subject (e.g., a mammal such as a human). In some embodiments, the subject is pre-selected by detecting an alteration in copy number and/or sequence of C4A and/or C4B polynucleotide relative to a reference. Thus, one embodiment is a method of treating a subject suffering from or susceptible to an autoimmune or inflammatory disorder or symptom thereof. The method includes the step of administering to the mammal a therapeutic amount of an amount of an agent herein sufficient to treat the disease or disorder or symptom thereof, under conditions such that the disease or disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method, such as the methods described herein).

The therapeutic methods of the invention (which include prophylactic treatment) in general comprise administration of a therapeutically effective amount of the agents herein to a subject (e.g., animal, human) in need thereof, including a mammal, particularly a human. Such treatment will be suitably administered to subjects, particularly humans, suffering from, having, susceptible to, or at risk for an autoimmune or inflammatory disease, disorder, or symptom thereof. In some embodiments, determination of those subjects “at risk” is made by an objective determination using the methods described herein.

In one embodiment, the invention provides a method of monitoring treatment progress. The method includes the step of determining a level of diagnostic marker (e.g., level of a polynucleotide or polypeptide of C4A and/or C4B) or diagnostic measurement (e.g., screen, assay) in a subject suffering from or susceptible to an autoimmune or inflammatory disease, or disorder or symptoms thereof, in which the subject has been administered a therapeutic or effective amount of a therapeutic agent described herein sufficient to treat the schizophrenia or symptoms thereof. The level of a polynucleotide or polypeptide of C4A and/or C4B determined in the method can be compared to known levels of a polynucleotide or polypeptide of C4A and/or C4B in either healthy normal controls or in other afflicted patients to establish the subject's disease status. In some embodiments, a level of a polynucleotide or polypeptide of C4A and/or C4B in a cerebrospinal fluid (CSF) sample obtained from the subject is determined. In some embodiments, a second level of a polynucleotide or polypeptide of C4A and/or C4B in the subject is determined at a time point later than the determination of the first level, and the two levels are compared to monitor the course of disease or the efficacy of the therapy. In certain embodiments, a pre-treatment level, sequence, or copy number of a polynucleotide or polypeptide of C4A and/or C4B in the subject is determined prior to beginning treatment according to this invention; this pre-treatment level of a polynucleotide or polypeptide of C4A and/or C4B can then be compared to the level of a polynucleotide or polypeptide of C4A and/or C4B in the subject after the treatment commences, to determine the efficacy of the treatment.

In particular embodiments, the agent is an agent that alters C4 expression or activity. In some embodiments, the agent is a complement inhibitor. FDA-approved complement inhibitors that are currently in use for other indications are suitable for use in the methods described herein and include, without limitation, Eculizumab/Soliris and Cetor/Sanquin. In some embodiments, the complement inhibitor is an anti-C1q antibody or fragment thereof (see, e.g., U.S. Patent Publication No. 2016/0159890). In other embodiments, the agent increases C4 expression or activity. In one embodiment, the agent (e.g., an expression vector containing a C4 polynucleotide sequence encoding C4) increases C4 expression.

### Therapeutic Agents Targeting C4A

In other aspects, the invention provides a method of treating an autoimmune disorder or inflammation by selectively interfering with the function of C4A polypeptide. In some embodiments, the interference with C4A polypeptide function is achieved using an antibody binding to C4A polypeptide. In some embodiments, the antibody specifically binds to C4A polypeptide, and does not bind C4B polypeptide. In certain embodiments, the antibody binds to both C4A and C4B polypeptide.

Antibodies can be made by any of the methods known in the art utilizing a polypeptide of the invention (e.g., C4A and C4B polypeptide), or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a polypeptide of the invention or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

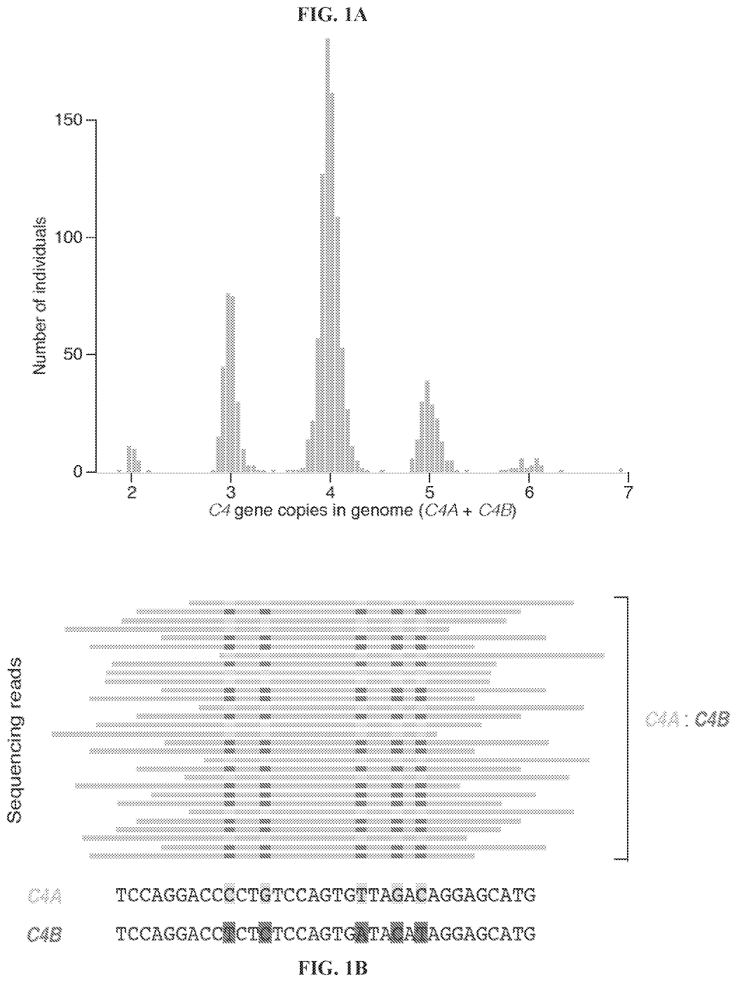

Without intending to be bound by theory, results herein indicate that therapeutically it might be advantageous to selectively interfere with C4A while leaving C4B function intact. This could be important because ideally one would not want to entirely block complement function in the body, since complement is important for protection from immune assault and from auto-immunity. Thus, in some embodiments, therapeutic antibodies that selectively bind to C4A polypeptide and not to C4B polypeptide are generated by exploiting the amino-acid sequence differences between C4A and C4B to identify epitopes for isotope-specific antibodies. In some embodiments, the amino acid sequence difference between C4A and C4B is that shown in FIG. 1B. Thus, in certain embodiments, the antibody specifically binds an epitope containing the sequence PCPVLD. In particular embodiments, the antibody does not bind an epitope containing the sequence LSPVIH.

### Pharmaceutical Compositions

The present invention features compositions useful for treating an autoimmune or inflammatory disorder in a subject. The administration of a composition comprising a therapeutic agent herein (e.g., an inhibitory nucleic acid inhibiting expression fo C4A polypeptide, or an antibody specifically binding to C4A polypeptide) for the treatment of an autoimmune or inflammatory disorder may be by any suitable means that results in a concentration of the therapeutic that, combined with other components, is effective in ameliorating, reducing, or stabilizing an autoimmune or inflammatory disorder in a subject. The composition may be administered systemically, for example, formulated in a pharmaceutically-acceptable buffer such as physiological saline. Routes of administration include, for example, intrathecal, subcutaneous, intravenous, interperitoneally, intramuscular, or intradermal injections that provide continuous, sustained levels of the agent in the patient. In particular embodiments, the composition comprising a therapeutic agent herein is administered intrathecally to a subject.

In certain embodiments, a chimeric molecule is generated comprising a fusion of an antibody or other therapeutic polypeptide with a protein transduction domain which targets the antibody or therapeutic polypeptide for delivery to various tissues and more particularly across the brain blood barrier, using, for example, the protein transduction domain of human immunodeficiency virus TAT protein (Schwarze et al., 1999, Science 285: 1569-72) or BBB peptide (Brainpeps® database; http://brainpeps.ugent.be/; Van Dorpe et al., Brain Structure and Function, 2012, 217(3), 687-718). Other polypeptides facilitating transport across the blood-brain-barrier, include without limitation, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, Syn-B, penetratin, a poly-arginine peptide, an angiopep peptide, and ANG1005.

The amount of the therapeutic agent to be administered varies depending upon the gender of the subject, the manner of administration, the age and body weight of the patient, and with the clinical symptoms of an autoimmune or inflammatory disorder. Generally, amounts will be in the range of those used for other agents used in the treatment of an autoimmune or inflammatory disorder, although in certain instances lower amounts will be needed because of the increased specificity of the agent. A composition is administered at a dosage that decreases effects or symptoms of an autoimmune or inflammatory disorder as determined by a method known to one skilled in the art.

The therapeutic agent (e.g., an agent herein) may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the liver; (v) formulations that allow for convenient dosing, such that doses are administered, for example, once every one or two weeks; and (vi) formulations that target schizophrenia using carriers or chemical derivatives to deliver the therapeutic agent to a particular cell type (e.g., cells in the brain). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered intrathecally or parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates schizophrenia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Inhibitory Nucleic Acid Therapy

Another therapeutic approach for treating or slowing progression of an autoimmune or inflammatory disorder is polynucleotide therapy using an inhibitory nucleic acid that inhibits expression of a C4A and/or C4B polynucleotide (in particular, a C4A polynucleotide). Thus, provided herein are inhibitory nucleic acid molecules, such as siRNA, that target C4A and/or C4B polynucleotide. Such nucleic acid molecules can be delivered to cells of a subject having schizophrenia. The nucleic acid molecules are delivered to the cells of a subject in a form in which they can be taken up so that therapeutically effective levels of the inhibitory nucleic acid molecules are introduced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, an inhibitory nucleic acid as described can be cloned into a retroviral vector and expression can be driven from its endogenous promoter, from the retroviral long terminal repeat, or from a promoter specific for a target cell type of interest. In some embodiments, the target cell type of interest is a neuron. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding inhibitory nucleic acid molecules that inhibit C4A and/or C4B expression.

Non-viral approaches can also be employed for the introduction of the therapeutic to a cell of a patient requiring treatment of an autoimmune or inflammatory disorder. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the inhibitory nucleic acid molecule is selectively expressed in a neuron. In some other embodiments, the inhibitory nucleic acid molecule is expressed in a neuron using a lentiviral vector. In still other embodiments, the inhibitory nucleic acid molecule is administered intrathecally. Selective targeting or expression of inhibitory nucleic acid molecules to a neuron is described in, for example, Nielsen et al., J Gene Med. 2009 July; 11(7):559-69. doi: 10.1002/jgm.1333.

### Screening Assays

The present invention further features methods of identifying modulators of a disease, particularly an autoimmune or inflammatory disorder, comprising identifying candidate agents that interact with and/or alter the level or activity of a polynucleotide or polypeptide of C4A or C4B.

Thus, in some aspects, the invention provides a method of identifying a modulator of an autoimmune or inflammatory disorder, comprising (a) contacting a cell or organism with a candidate agent, and (b) measuring a level of polynucleotide or polypeptide of C4A or C4B in the cell relative to a control level. An alteration in the level of C4A or C4B polypeptide or polynucleotide indicates the candidate agent is a modulator of schizophrenia. In particular, a decrease in the level of C4A polynucleotide or polypeptide indicates the candidate agent is an inhibitor of C4A. In some embodiments, the cell or organism is a recombinant cell or recombinant organism that overexpresses C4A polynucleotide or polypeptide.

Methods of measuring or detecting activity and/or levels of the polypeptide or polynucleotide are known to one skilled in the art. Polynucleotide levels may be measured by standard methods, such as quantitative PCR, Northern Blot, microarray, mass spectrometry, and in situ hybridization. Standard methods may be used to measure polypeptide levels, the methods including without limitation, immunoassay, ELISA, western blotting using an antibody that binds the polypeptide, and radioimmunoassay.

In some embodiments, the C4A polypeptide is fused to a detectable label (e.g., a fluorescent reporter polypeptide). Level(s) of C4A polypeptide in a cell contacted with a candidate agent can then be easily monitored by measuring fluorescence of the reporter polypeptide.

### Kits

The invention provides kits, e.g., for treating an autoimmune or inflammatory disorder in a subject and/or identifying a subject having or at risk of developing an autoimmune or inflammatory disorder. A kit of the invention provides a capture reagent (e.g., a primer or hybridization probe specifically binding to a C4A or C4B polynucleotide) for measuring relative expression level, copy number, and/or a sequence of a marker (e.g., C4A or C4B). In other embodiments, the kit further includes reagents suitable for DNA sequencing or copy number analysis of C4A and/or C4B.

In one embodiment, the kit includes a diagnostic composition comprising a capture reagent detecting at least one marker selected from the group consisting of a C4A polynucleotide and a C4B polynucleotide. In one embodiment, the capture reagent detecting a polynucleotide of C4A or C4B is a primer or hybridization probe that specifically binds to a C4A or C4B polynucleotide.

In some embodiments, the kit comprises a sterile container which contains a therapeutic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for using the diagnostic agents and/or administering the therapeutic agents of the invention. In particular embodiments, the instructions include at least one of the following: description of the therapeutic agent; dosage schedule and administration for reducing symptoms; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

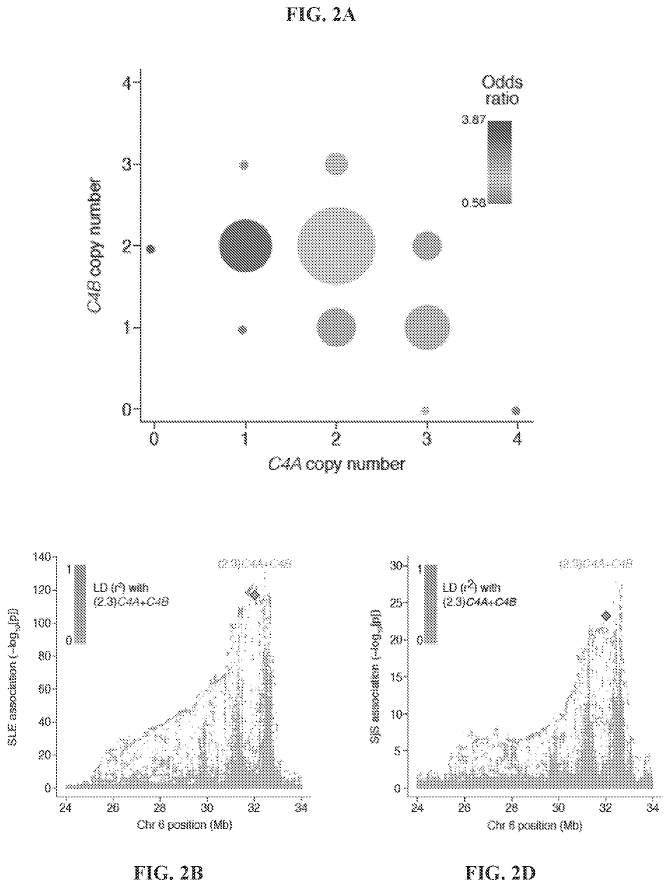

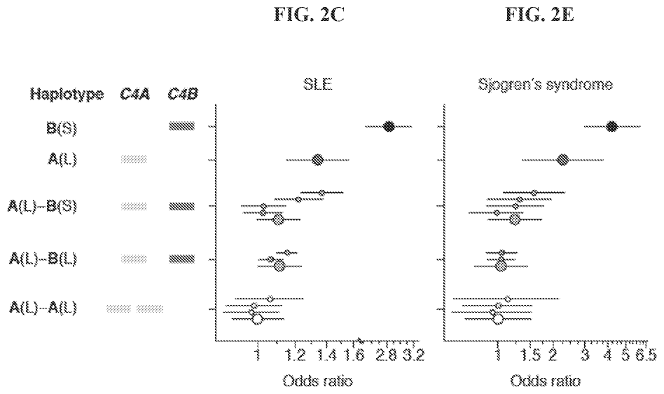

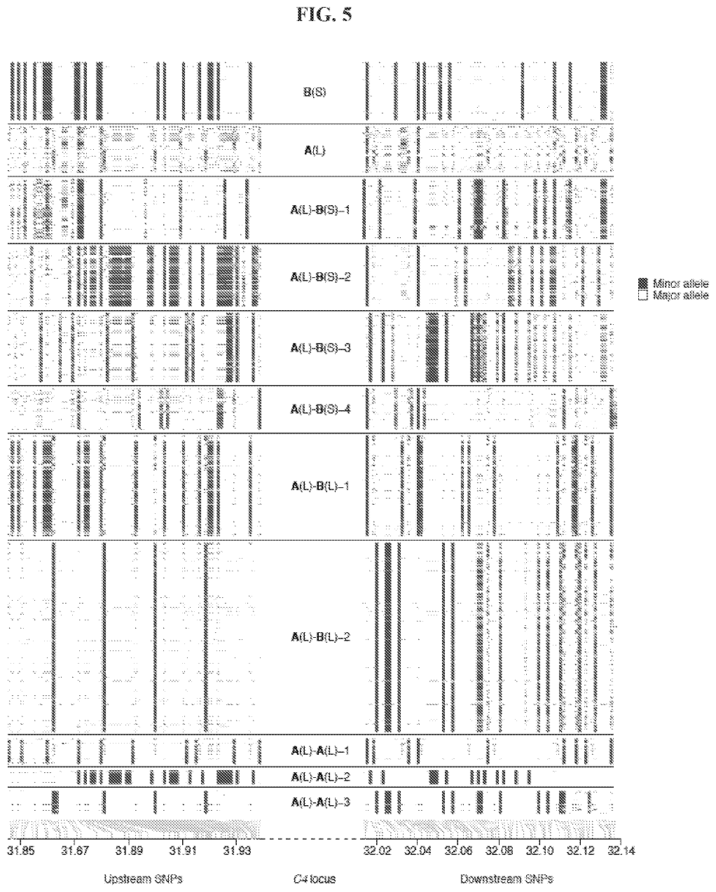

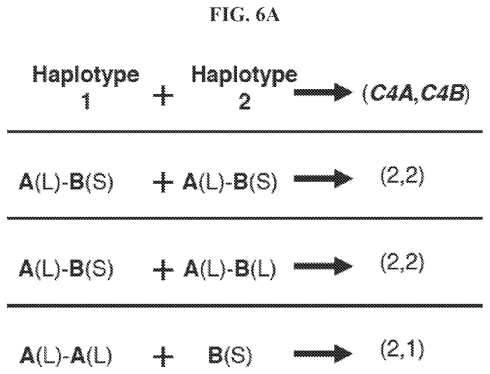

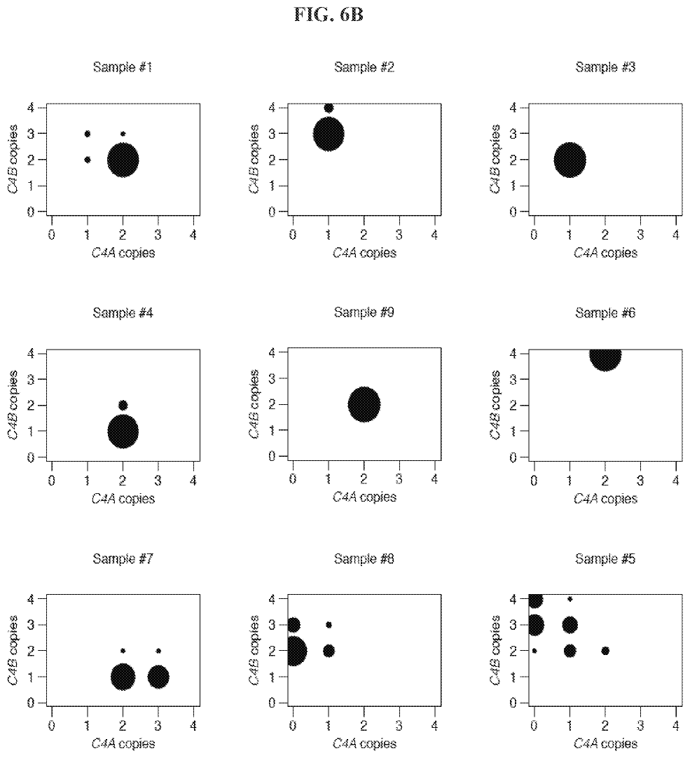

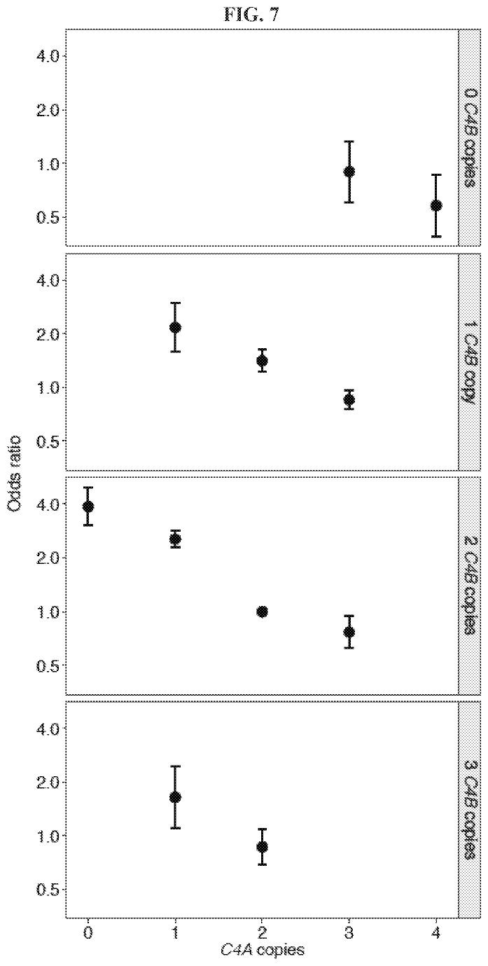

The complex genetic variation at C4—arising from many alleles with different numbers of C4A and C4B genes—has been challenging to analyze in large cohorts. A recently feasible approach to this problem is based on imputation: people share long haplotypes with the same combinations of SNP and C4 alleles, such that C4A and C4B gene copy numbers can be imputed from SNP data. To analyze C4 in large cohorts, a method to identify C4 alleles from whole-genome sequence (WGS) data (FIGS. 1A and 1B) was developed. WGS data were analyzed from 1,265 individuals (from the Genomic Psychiatry Cohort) to create a large multi-ancestry panel of 2,530 reference haplotypes of MHC SNPs and C4 alleles (FIG. 5)—ten times more than in earlier work. SNP data from the largest SLE genetic association study were analyzed (ImmunoChip 6,748 SLE cases and 11,516 controls of European ancestry) (FIGS. 6A and 6B), imputing C4 alleles to estimate the SLE risk associated with common combinations of C4A and C4B gene copy numbers (FIG. 2A).

Groups with the eleven most common combinations of C4A and C4B gene copy number exhibited 7-fold variation in their risk of SLE (FIG. 2A and FIG. 7). The relationship between SLE vulnerability and C4 gene copy number exhibited consistent, logical patterns across the 11 genotype groups. For each C4B copy number, greater C4A copy number associated with reduced SLE risk (FIG. 2A, FIG. 7). For each C4A copy number, greater C4B copy number associated with more modestly reduced risk (FIG. 2A). Logistic-regression analysis estimated that the protection afforded by each copy of C4A (OR: 0.54; 95% CI: [0.51, 0.57]) was equivalent to that of 2.3 copies of C4B (OR: 0.77; 95% CI: [0.71,0.82]). An initial C4-derived risk score was calculated as 2.3 times the number of C4A genes, plus the number of C4B genes, in an individual's genome. Despite clear limitations of this risk score—it is imperfectly imputed from flanking SNP haplotypes (r2=0.77, FIG. 13) and only approximates C4-derived risk by using a simple, linear model (to avoid over-fitting the genetic data)—SNPs across the MHC locus tended to associate with SLE in proportion to their level of LD with this risk score (FIG. 2B).

Based on these results, it was considered whether other autoimmune disorders with similar patterns of genetic association at the MHC locus might also be driven in part by C4 variation. Sjögren's syndrome (SjS) is a heritable (54%) systemic autoimmune disorder of exocrine glands, characterized primarily by dry eyes and mouth with other systemic effects. At a protein level, SjS is (like SLE) characterized by diverse autoantibodies, including antinuclear antibodies targeting ribonucleoproteins, and 135 hypocomplementemia. The largest source of common genetic risk for SjS lies in the MHC locus, with associations to the same haplotype(s) as in SLE and with heterogeneous HLA associations in different ancestries. C4 alleles were imputed into existing SNP data from a European-ancestry SjS case-control cohort (673 cases and 1153 controls). As in SLE, logistic-regression analyses found both C4A copy number (OR: 0.41; 95% CI: [0.34, 0.49]) and C4B copy number (OR: 0.67; 95% CI: [0.53, 0.86]) to be protective against SjS. The risk-equivalent ratio of C4B to C4A gene copies was similar in SjS and SLE (about 2.3 to 1); also, as with SLE, nearby SNPs associated with SjS in proportion to their LD with a C4-derived risk score ((2.3)C4A+C4B) (FIG. 2D). The distribution of SjS risk across the 170 individual C4 alleles and haplotypes revealed a pattern that (as in SLE) supported greater protective effect from C4A than C4B, and little effect of flanking SNP haplotypes (FIG. 2E).

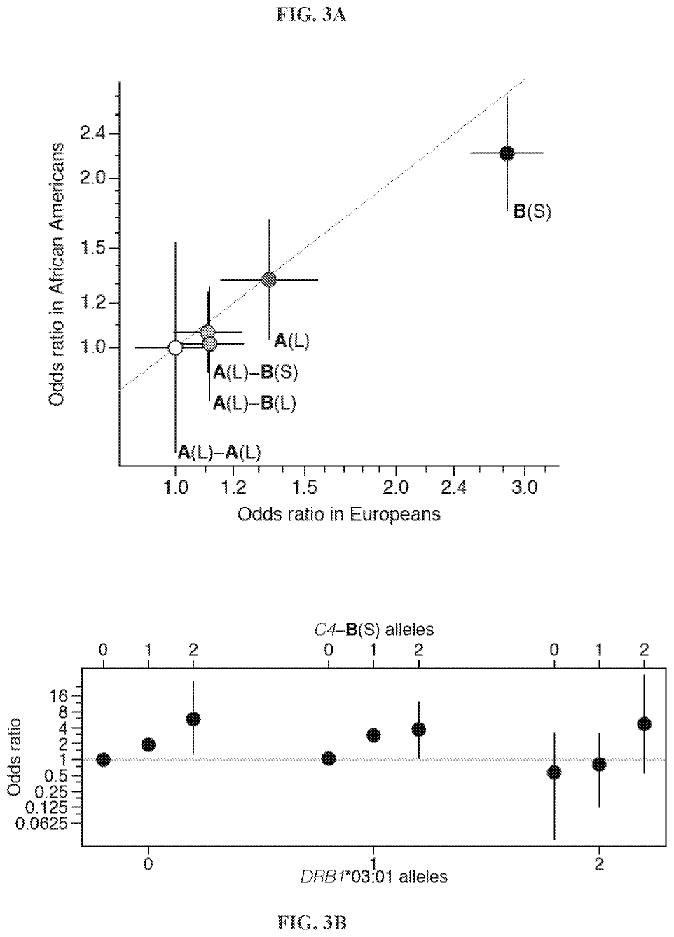

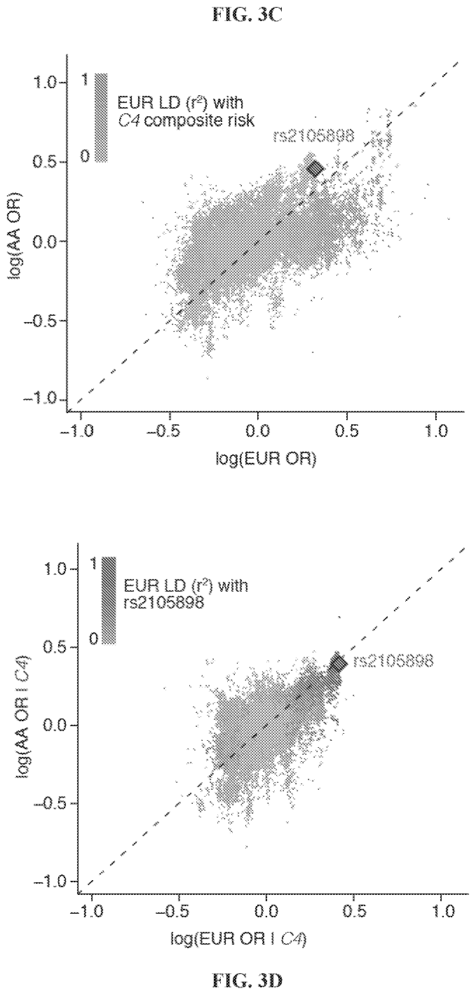

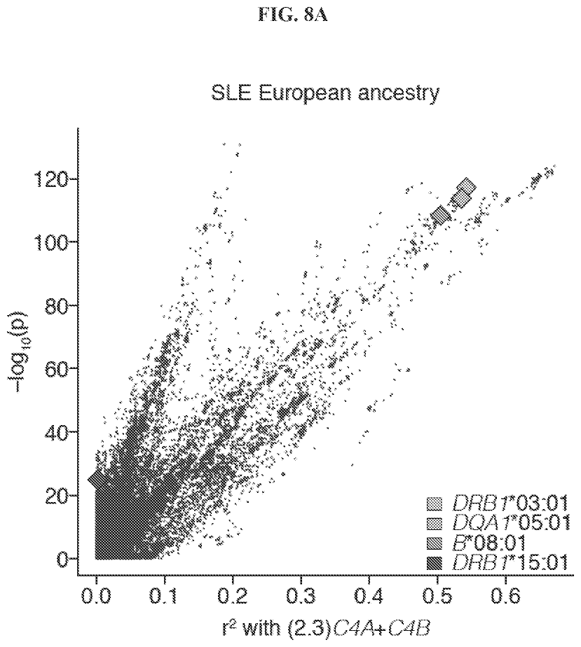

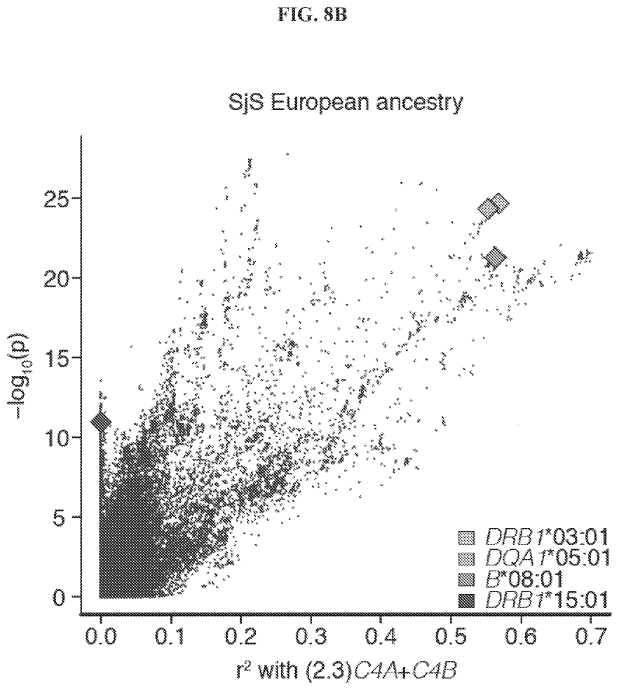

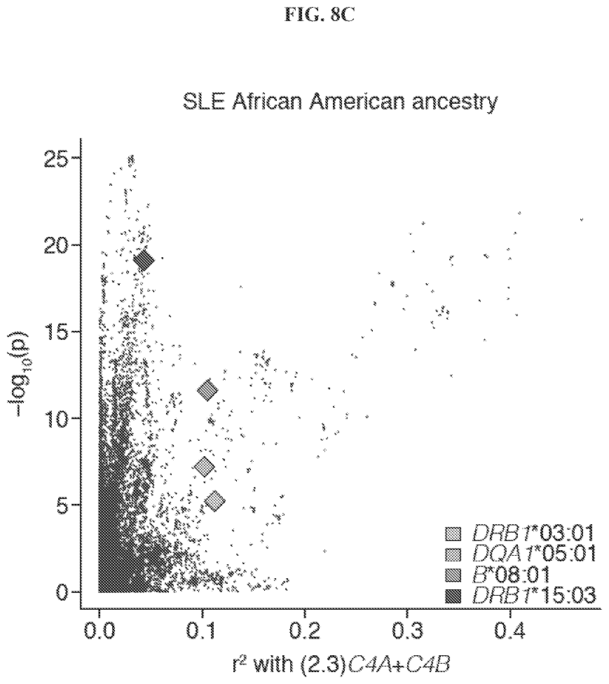

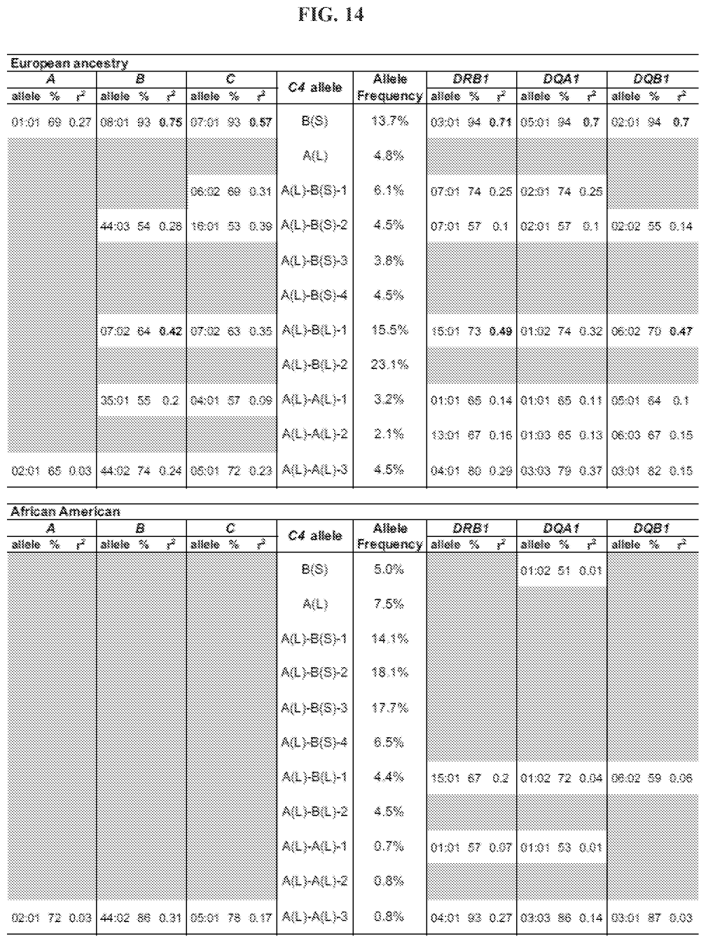

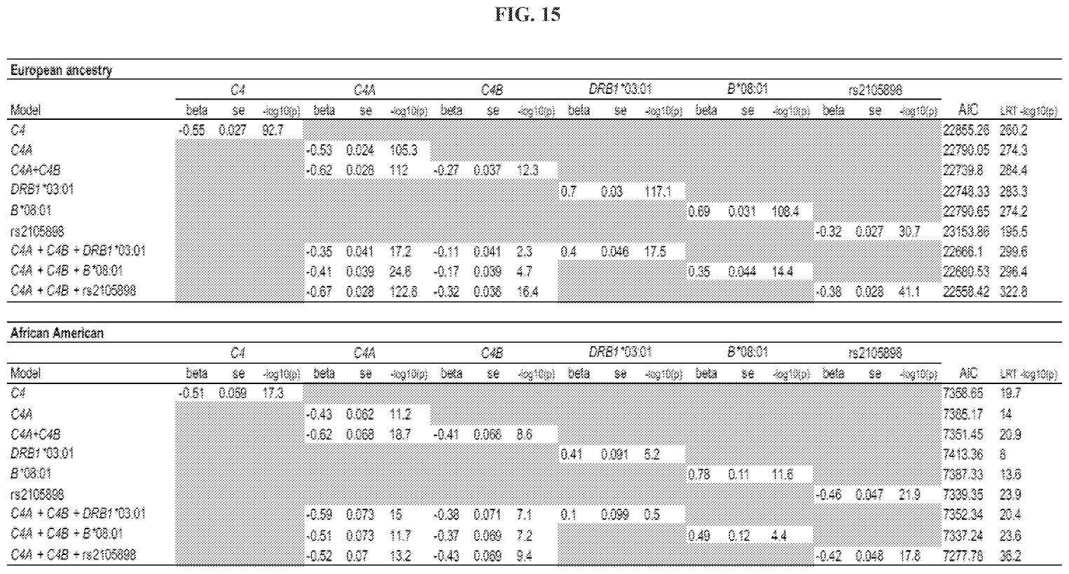

The association of SLE and SjS with C4 gene copy number has long been attributed to the HLA 175 DRB1*03:01 allele. In European populations, DRB1*03:01 is in strong LD (r2=0.71) with the common C4-B(S) allele, which lacks any C4A gene and is the highest-risk C4 allele in the analysis described herein (FIG. 2C); many MHC SNPs associated with SLE and SjS in proportion to their LD correlations with both C4 and DRB1*03:01 (FIGS. 8A and 8B). Cohorts with other ancestries can have recombinant haplotypes that disambiguate the contributions of alleles that are in LD in Europeans. Among African Americans, it was found that common C4 alleles exhibited far less LD with HLA alleles; in particular, the LD between C4-B(S) and DRB1*03:01 was low (r2=0.10) (FIG. 14). Thus, genetic data from an African American SLE cohort (1,494 cases, 5,908 controls) made it possible to distinguish between these potential genetic effects. Joint association analysis of C4A, C4B, and DRB1*0301 implicated C4A (p<10-14) and C4B (p<10-5) but not DRB1*0301 (p=0.29) (FIG. 15). Each C4 allele associated with effect sizes of similar magnitude on SLE risk in Europeans and African Americans (FIG. 3A). An analysis specifically of combinations of C4-B(S) and DRB1*03:01 allele dosages in African Americans showed that C4-B(S) alleles consistently increased SLE risk regardless of DRB1*03:01 status, whereas DRB1*03:01 had no consistent effect when controlling for C4-B(S) (FIG. 3B). Although C4 alleles had less LD with nearby variants on African American than on European haplotypes, SNPs associated with SLE in proportion to LD correlations with C4 in African Americans as well (FIG. 8C).

Other potential contributions of the MHC locus to SLE risk were determined by accounting for contributions from C4. SNPs across the MHC locus display very different associations with SLE in Europeans and African Americans, though the SNPs with European-specific associations tend to have strong LD to C4 in Europeans (FIG. 3C). To control for C4 genotypes, many of which exhibit strong LD across the MHC locus in Europeans (FIG. 5), the association data for C4-derived risk was adjusted using a more-complete C4-derived risk score derived from the genotype-group risk measurements in FIG. 2A. Once adjusted for C4 effects, the residual association signals in the two populations became strongly correlated (FIG. 3D). Both populations also pointed to the same small haplotype of two variants as the most likely driver of an additional genetic effect independent of C4 (FIG. 3D and Example 3). The two variants defining this short haplotype reside within the XL9 regulatory region, a well-studied region of open chromatin that contains abundant chromatin marks characteristic of active enhancers and transcription factor binding sites (Example 3). One of these variants, rs2105898, disrupts a binding site for ZNF143, which anchors interactions of distal enhancers with gene promoters (Example 3). Data from the GTEx Consortium (v7) included 227 instances (gene/tissue pairs) in which this haplotype associated with elevated (HLA-DRB1, -DRB5, -DQA1, and -DQB1) or reduced (HLA-DRB6, -DQA2, and -DQB2) expression of an HLA class II gene with at least nominal (p<10-4) significance. Some of the strongest associations at each gene (p<10⁻⁸ to 10⁻⁷⁶) were in whole blood, but expression QTLs elsewhere can also reflect the presence of blood and immune cells within those tissues. (Although eQTL analyses of HLA genes may be affected by read-alignment artifacts in these genes' hyperpolymorphic domains, most such observed signals are robust after adjusting for individual HLA alleles.)

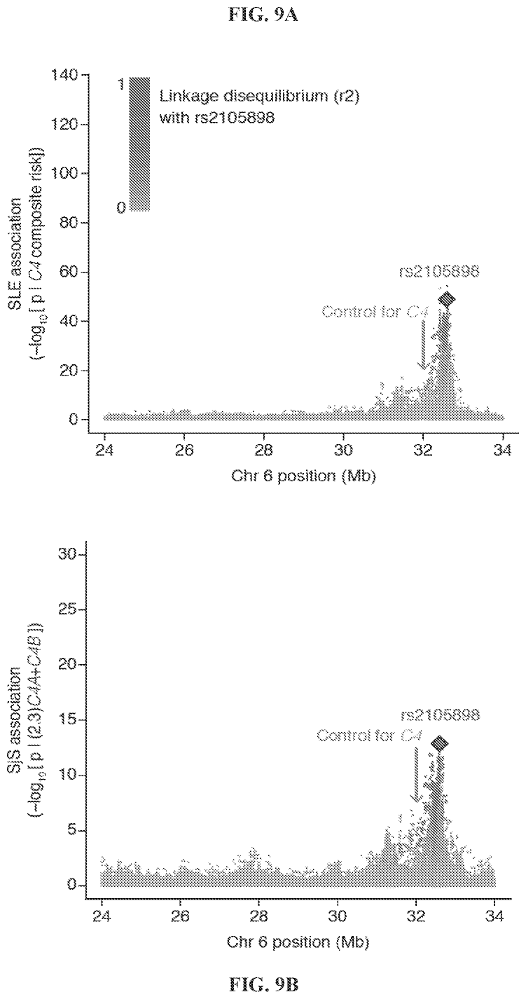

The haplotype with elevated expression of HLA-DRB1, -DRB5, -DQA1, and -DQB1 (allele frequency 0.20 among Europeans, 0.22 among African Americans) associated with increased SLE risk (odds ratio) of 1.52 (95% CI: 1.44-1.61; p<10⁻⁴⁸) in Europeans and 1.49 (95% CI: 1.35-1.63; p<10⁻¹⁶) in African Americans in analyses adjusting for C4 effects. The risk haplotype was in strong LD with DRB1*15:01 in Europeans and DRB1*15:03 in African Americans, which may explain earlier findings of population-specific associations with DRB1*15:01 in Europeans and DRB1*15:03 in African Americans. The risk haplotype tagged by rs2105898 tended to be on low-risk C4 haplotypes in Europeans, a relationship that may have made both genetic influences harder to recognize in earlier work; controlling for either rs2105898 or C4 (FIG. 9A) greatly increased the association of SLE with the other genetic influence (FIG. 15). Controlling for the simpler (2.3)C4A+C4B model in SNP associations with SjS (as precision of estimates of individual alleles were low due to sample size) also pointed strongly to the same haplotype, with the same allele of rs2105898 associating in the same direction but larger effect (OR: 1.96; 95% CI: 1.64-2.34) as compared to SLE (FIG. 9B).

Alleles at C4 that increase dosage of C4A, and to a lesser extent C4B, appear to protect strongly against SLE and SjS (FIGS. 2A-2C); by contrast, alleles that increase expression of C4A in the brain are more common among individuals with schizophrenia. These same illnesses exhibit striking, and opposite, sex differences: SLE and SjS are nine times more common among women of childbearing age than among men of a similar age, whereas in schizophrenia, women exhibit less severe symptoms, more frequent remission of symptoms, lower relapse rates, and lower overall incidence. Hence, the possibility that the effects of C4 alleles on the risk of each disease might also differ between men and women was evaluated.

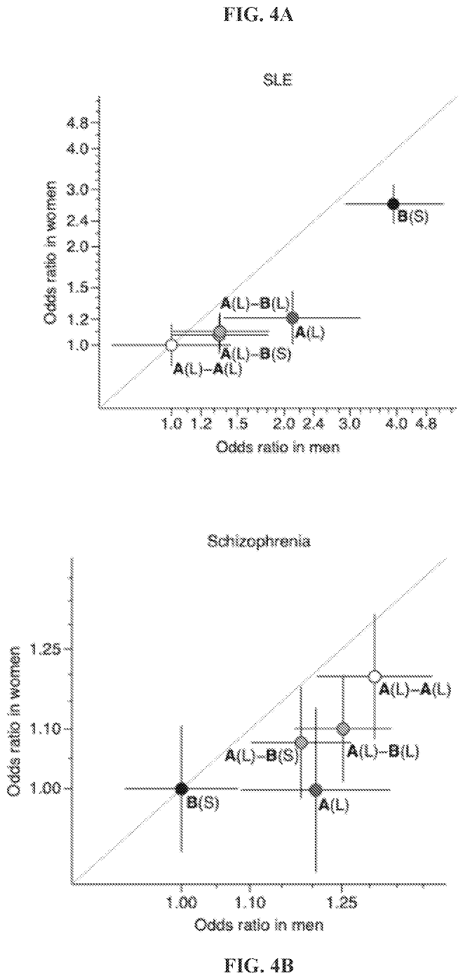

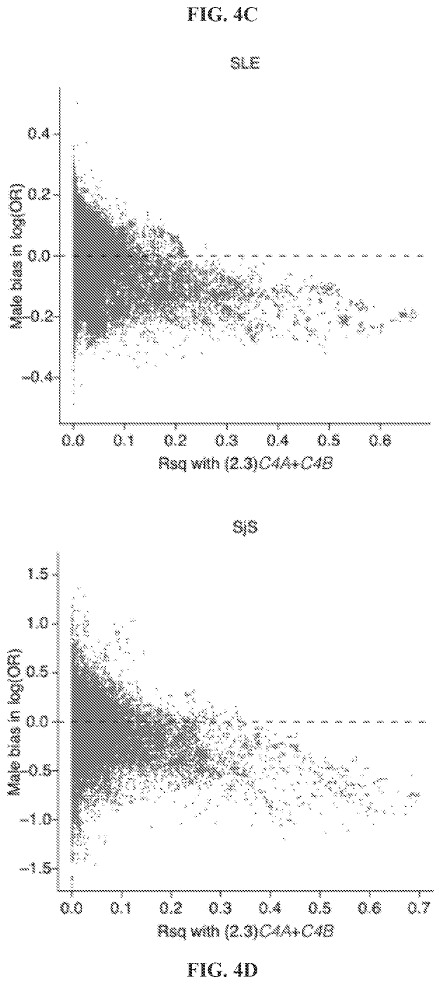

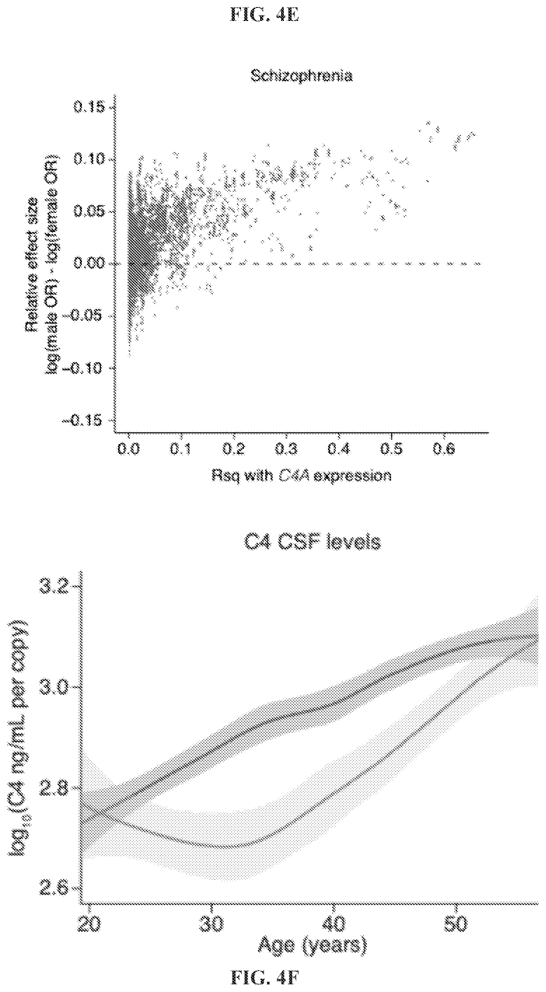

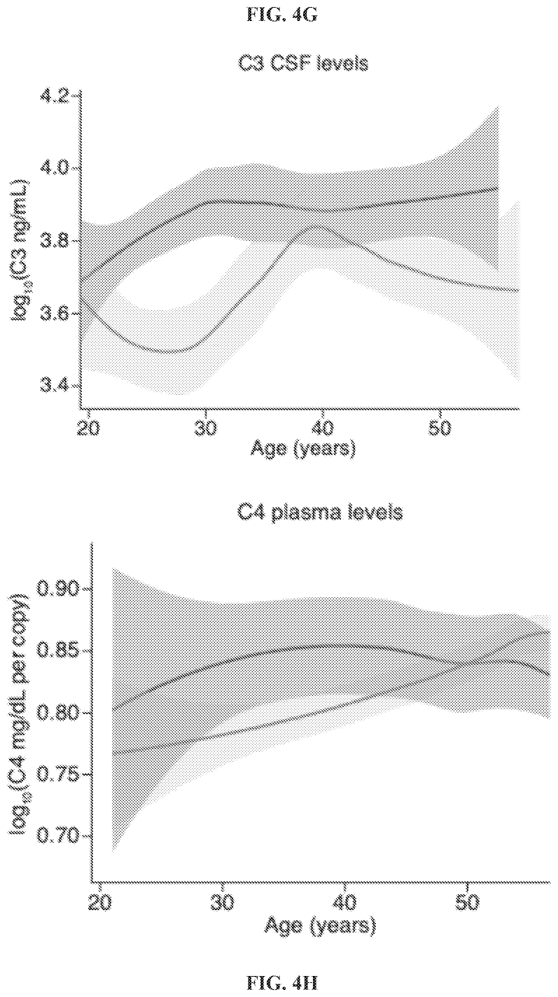

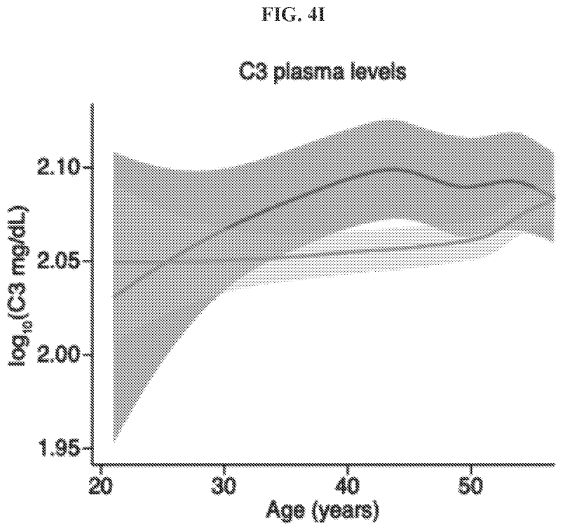

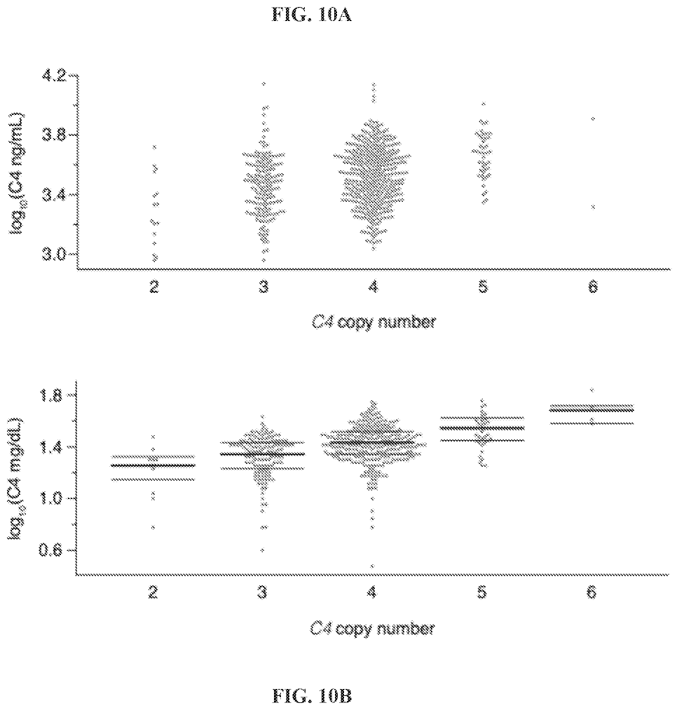

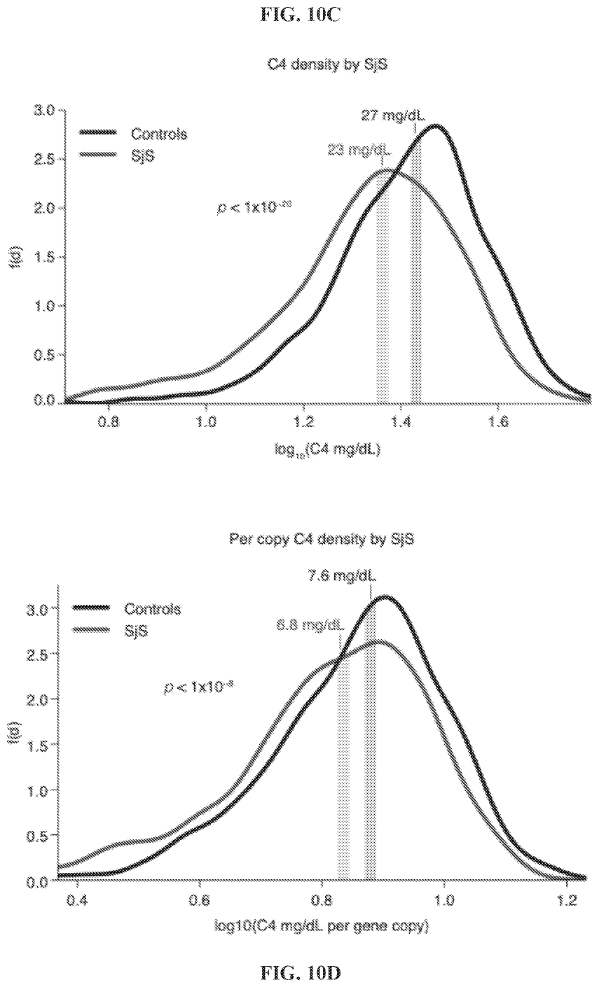

Analysis indicated that the effects of C4 alleles in both lupus and schizophrenia were stronger in men. When a sex-by-C4 interaction term was included in association analyses, this term was significant for both SLE (p<0.01) and schizophrenia (p<0.01), indicating larger C4 effects in men for both disorders. (Analysis of SjS had limited power due to the small number of men affected by SjS—60 of the 673 cases in the cohort—but pointed to the same direction of effect at p=0.07). For both SLE and schizophrenia, the individual C4 alleles consistently associated with stronger effects in men than women (FIGS. 4A and 4B). These relationships explained previously reported sex biases in SNP associations across the MHC locus (FIGS. 4C-4E). The stronger effects of C4 alleles on male relative to female risk could arise from sex differences in C4 RNA expression, C4 protein levels, or downstream responses to C4. Analysis of RNA expression in 45 tissues, using data from GTEx, identified no sex differences in C4 RNA expression. C4 protein was then analyzed in cerebrospinal fluid (CSF) from two panels of adult research participants (n=589 total) in whom C4 gene copy number had also been measured by direct genotyping or imputation. CSF C4 protein levels correlated strongly with C4 gene copy number (p<10⁻¹⁰, FIG. 10A); therefore, C4 protein measurements were normalized to the number of C4 gene copies. CSF from adult men contained on average 27% more C4 protein per C4 gene copy than CSF from women (meta-analysis p=9.9×10⁻⁶, FIG. 4F). C4 acts by activating the complement component 3 (C3) protein, promoting C3 deposition onto targets in tissues. CSF levels of C3 protein were also on average 42% higher among men than women (meta-analysis p=7.5×10⁻⁷, FIG. 4G). The elevated concentrations of C3 and C4 proteins in CSF of men parallel earlier findings showing that, in plasma, C3 and C4 are also present at higher levels in men than women. The large sample size (n>50,000) of the plasma studies allows sex differences to be further analyzed as a function of developmental age. Both men and women undergo age-dependent elevation of C4 and C3 levels in plasma, but this occurs early in adulthood (age 20-30) in men and closer to menopause (age 40-50) in women, with the result that male-female differences in complement protein levels are observed primarily during the reproductive years (ages 20-50). These findings were replicated using measurements of C3 and (gene copy number-corrected) (FIG. 10B) C4 protein in plasma from adults, finding (as in the earlier plasma studies and in CSF) that these differences are most pronounced during the reproductively active years of adulthood (ages 20-50) (FIGS. 4H and 4I). SjS patients were also observed to have lower C4 serum levels than controls (p<1×10⁻²⁰, FIG. 10C) even after correcting for C4 gene copy number (p<1×10⁻⁸, FIG. 10D), suggesting that hypocomplementemia in SjS is not simply due to C4 genetics but also reflects disease effects on ambient complement levels, for example, due to complement consumption. The ages of pronounced sex difference in complement levels corresponded to the ages at which men and women differ in disease incidence: in schizophrenia, men outnumber women among cases incident in early adulthood, but not among cases incident after age 40; in SLE, women greatly outnumber men among cases incident during the child-bearing years, but not among cases incident after age 50 or during childhood; in SjS, the large relative vulnerability of women declines in magnitude after age 50.

The results described herein indicate that the MHC locus shapes vulnerability in lupus and SjS—two of the three most common rheumatic autoimmune diseases—in a very different way than in type I diabetes, rheumatoid arthritis, and celiac disease. In those diseases, precise interactions between specific HLA alleles and specific autoantigens determine risk. In SLE and SjS, however, the genetic variation implicated here points instead to the continuous, chronic interaction of the immune system with very many potential autoantigens. Because complement facilitates the rapid clearance of debris from dead and injured cells, elevated levels of C4 protein likely attenuate interactions between the adaptive immune system and ribonuclear self-antigens at sites of cell injury, pre-empting the development of autoimmunity. The additional C4-independent genetic risk effect described here (associated with rs2105898) may also affect autoimmunity broadly, rather than antigen-specifically, by regulating expression of many HLA class II genes (including DRB1, DQA1, and DQB1). Mouse models of SLE indicate that once tolerance is broken for one self-antigen, autoreactive germinal centers generate B cells targeting other self-antigens; such “epitope spreading” could lead to autoreactivity against many related autoantigens, regardless of which antigen(s) are involved in the earliest interactions with immune cells. The genetic findings described herein address the development of SLE and SjS rather than complications that arise in any specific organ. A few percent of SLE patients develop neurological complications that can include psychosis; though psychosis is also a symptom of schizophrenia, neurological complications of SLE do not resemble schizophrenia more broadly, and likely have a different etiology.

The same C4 alleles that increase vulnerability to schizophrenia appeared to protect strongly against SLE and SjS. This pleiotropy will be important to consider in efforts to engage the complement system therapeutically. The complement system contributed to these pleiotropic effects more strongly in men than in women. Moreover, though the allelic series at C4 allowed human genetics to establish dose-risk relationships for C4, sexual dimorphism in the complement system also extended to complement component 3 (C3). Why and how biology has come to create this sexual dimorphism in the complement system in humans presents interesting questions for immune and evolutionary biology.

### Example 2—Creation of a C4 Reference Panel from Whole-Genome Sequence Data (Methods)

A reference panel for imputation of C4 structural haplotypes was constructed using whole-genome sequencing data for 1265 individuals from the Genomic Psychiatry Cohort. The reference panel included individuals of diverse ancestry, including 765 Europeans, 250 African Americans, and 250 people of reported Latino ancestry.

The diploid C4 copy number, and separately the diploid copy number of the contained HERV segment, were estimated using Genome STRiP (Genome STRucture In Populations). Briefly, Genome STRiP carefully calibrates measurements of read depth across specific genomic segments of interest by estimating and normalizing away sample-specific technical effects, such as the effect of GC content on read depth (estimated from the genome-wide data). To estimate C4 copy number, the segments 6:31948358-31981050 and 6:31981096-32013904 (hg19) were genotyped for total copy number; the intronic HERV segments that distinguish short (S) from long (L) C4 gene isotypes were masked. For the HERV region, segments 6:31952461-31958829 and 6:31985199-31991567 (hg19) were genotyped for total copy number. Across the 1,265 individuals, the resultant locus-specific copynumber estimates exhibited a strongly multi-modal distribution (FIG. 1A) from which individuals' total C4 copy numbers could be readily inferred.

The ratio of C4A to C4B genes were then estimated in each individual genome. To do this, reads mapping to the paralogous sequence variants that distinguish C4A from C4B (hg19 coordinates 6:31963859-31963876 and 6:31996597-31996614) in each individual were extracted, and reads across the two sites were combined. Only reads that aligned to one of these segments in its entirety were included. The number of reads matching the canonical active site sequences for C4A (CCC TGT CCA GTG TTA GAC) and C4B (CTC TCT CCA GTG ATA CAT) were then counted. These counts were combined with the likelihood estimates of diploid C4 copy number (from Genome STRiP) to determine the maximum likelihood combination of C4A and C4B in each individual. The genotype quality of the C4A and C4B estimate was estimated from the likelihood ratio between the most likely and second most likely combinations.

To phase the C4 haplotypes, the GenerateHaploidCNVGenotypes utility in Genome STRiP was first used to estimate haplotype-specific copy-number likelihoods for C4 (total C4 gene copy number), C4A, C4B, and HERV using the diploid likelihoods from the prior step as input. Default parameters for GenerateHaploidCNVGenotypes were used, plus -genotypeLikelihoodThreshold 0.0001. The output was then processed by the GenerateCNVHaplotypes utility in Genome STRiP to combine the multiple estimates into likelihood estimates for a set of unified structural alleles. GenerateCNVHaplotypes was run with default parameters, plus-defaultLogLikelihood −50, -unknownHaplotypeLikelihood −50, and -sampleHaplotypePriorLikelihood 2.0. The resultant VCF was phased using Beagle 4.1 (beagle_4.1_27Jul16.86a) in two steps: first, performing genotype refinement from the genotype likelihoods using the Beagle gtgl= and −maxlr=1000000 parameters, and then running Beagle again on the output file using gt= to complete the phasing.

Previous work of the inventors suggested that several C4 structures segregate on different haplotypes, and probably arose by recurrent mutation on different haplotype backgrounds. The GenerateCNVHaplotypes utility requires as input an enumerated set of structural alleles to assign to the samples in the reference cohort, including any structurally equivalent alleles, with distinct labels to mark them as independent, plus a list of samples to assign (with high likelihood) to specific labeled input alleles to disambiguate among these recurrent alleles. The selection of the set of structural alleles to be modeled, along with the labeling strategy, is important to the methodology described here, and the performance of the reference panel. In the reference panel, each input allele represents a specific copy number structure and optionally includes a label that differentiates the allele from other independent alleles with equivalent structure. The notation <H_n_n_n_n_L> is used to identify each allele, where the four integers following the H are, respectively, the (redundant) haploid count of the total number of C4 copies, C4A copies, C4B copies and HERV copies on the haplotype. For example, <H_2_1_1_1> was used to represent the “AL-BS” haplotype. The optional final label L is used to distinguish potentially recurrent haplotypes with otherwise equivalent structures (under the model) that should be treated as independent alleles for phasing and imputation. To build the reference panel, a large number of potential sets of structural alleles and methods for assigning labels to potentially recurrent alleles were experimentally evaluated. For each evaluation, a reference panel was built using the 1265 reference samples, and then the performance of the panel was evaluated via cross-validation, leaving out 10 different samples in each trial (5 samples in the last trial) and imputing the missing samples from the remaining samples in the panel. The imputed results for all 1265 samples were then compared to the original diploid copy number estimates to evaluate the performance of each candidate reference panel (FIG. 13).

Using this procedure, a final panel for downstream analysis was selected that used a set of 29 structural alleles representing 16 distinct allelic structures (as listed in the reference panel VCF file). Each allele contained from one to three copies of C4. Three allelic structures (AL-BS, AL-BL, and AL-AL) were represented as a set of independently labeled alleles with 9, 3, and 4 labels, respectively.

To identify the number of labels to use on the different alleles and the samples to “seed” the alleles, “spider plots” of the C4 locus were generated based on initial phasing experiments run without labeled alleles, and then the resulting haplotypes were clustered in two dimensions based on the Y-coordinate distance between the haplotypes on the left and right sides of the spider plot. Clustering was based on visualizing the clusters (FIG. 5) and then manually choosing both the number of clusters (labels) to assign and a set of confidently assigned haplotypes to use to “seed” the clusters in GenerateCNVHaplotypes. This procedure was iterated multiple times using cross-validation, as described above, to evaluate the imputation performance of each candidate labeling strategy.

Within the data set used to build the reference panel, there is evidence for individuals carrying seven or more diploid copies of C4, which implies the existence of (rare) alleles with four or more copies of C4. In the experiments described here, attempting to add additional haplotypes to model these rare four-copy alleles reduced overall imputation performance. Consequently, all downstream analyses were conducted using a reference panel that models only alleles with up to three copies of C4. In the future, larger reference panels might benefit from modeling these rare four-copy alleles.

### Genetic Data for SLE

For analysis of systemic lupus erythematosus (SLE), collection and genotyping of the European-ancestry cohort (6,748 cases, 11,516 controls, genotyped by ImmunoChip) were essentially as described in Langefeld, C. D. et al., 2017, Nat Commun 8, 16021, doi:10.1038/ncomms16021. Collection and genotyping of the African-American cohort (1,494 cases, 5,908 controls, genotyped by OmniExpress) were essentially as described in Hanscombe, K. B. et al., 2018, Hum Mol Genet 27, 3813-3824, doi:10.1093/hmg/ddy280.

### Genetic Data for SjS

For analysis of Sjögren's syndrome (SjS), collection and genotyping of the European-ancestry cohort (673 cases, 1,153 controls, genotyped by Omni2.5) were essentially as described in Taylor, K. E. et al., 2017, Arthritis Rheumatol 69, 1294-1305, doi:10.1002/art.40040, and available in dbGaP under study accession number phs000672.v1.p1. 16

### Genetic Data for Schizophrenia

The schizophrenia analysis made use of genotype data from 40 cohorts of European ancestry (28,799 cases, 35,986 controls) made available by the Psychiatric Genetics Consortium (PGC), (Schizophrenia Working Group of the Psychiatric Genomics, C. Biological insights from 108 schizophrenia-associated genetic loci. Nature 511, 421-427, doi:10.1038/nature13595 (2014). Genotyping chips used for each cohort are listed in Supplementary Table 3 of that study.

### Imputation of C4 Alleles

The reference haplotypes described above were used to extend the SLE, SjS, or schizophrenia cohort SNP genotypes by imputation. SNP data in VCF format were used as input for Beagle v4.1 for imputation of C4 as a multi-allelic variant. Within the Beagle pipeline, the reference panel was first converted to bref format. From the cohort SNP genotypes, only those SNPs from the MHC region (chr6:24-34 Mb on hg19) that were also in the haplotype reference panel were used. The conform-gt tool was used to perform strandflipping and filtering of specific SNPs for which strand remained ambiguous. Beagle was run using default parameters with two key exceptions: the GRCh37 PLINK recombination map was used, and the output was set to include genotype probability (i.e., GP field in VCF) for correct downstream probabilistic estimation of C4A and C4B joint dosages.

### Imputation of HLA Alleles

For HLA allele imputation, sample genotypes were used as input for the R package HIBAG47. For both European ancestry and African American cohorts, publicly available multi-ethnic reference panels generated for the most appropriate genotyping chip (i.e. Immunochip for European ancestry SLE cohort, Omni 2.5 for European ancestry SjS cohort, and OmniExpress for African American SLE cohort) were used. Default parameters were used for all settings. All class I and class II HLA genes were imputed. Output haplotype posterior probabilities were summed per allele to yield diploid dosages for each individual.

### Associating Single and Joint C4 Structural Allele Dosages to SLE and SjS in European Ancestry Individuals

The analysis described above yields dosage estimates for each of the common C4 structural haplotypes (e.g., AL-BS, AL-AL, etc.) for each genome in each cohort. In addition to performing association analysis on these structures (FIG. 1B), an association analysis was also performed on the dosages of each underlying C4 gene isotype (i.e. C4A, C4B, C4L, and C4S). These dosages were computed from the allelic dosage (DS) field of the imputation output VCF simply by multiplying the dosage of a C4 structural haplotype by the number of copies of each C4 isotype that haplotype contains (e.g., AL-BL contains one C4A gene and one C4B gene).

C4 isotype dosages were then tested for disease association by logistic regression, with the inclusion of four available ancestry covariates derived from genome-wide principal component analysis (PCA) as additional independent variables, PCc,

logit(θ)˜β₀+β₁C4+Σ_{c}β_{c}PC_{c}+ε (1)

where θ=E[SLE|X]. For SjS, the model instead included two available multiethnic ancestry covariates from dbGaP that correlated strongly with European-specific ancestry covariates (specifically, PC5 and PC7) and 17 smoking status as independent variables. Coefficients for relative weighting of C4A and C4B dosages were obtained from a joint logistic regression,

logit(θ)˜β₀+β₁C4A+β2C4B+Σ_{c}β_{c}PC_{c}+ε (2)

The values per individual of β₁C4A+β₂C4B were used as a combined C4 risk term for estimating both association strength (FIG. 7) as well as evaluating the relationship between the strength of nearby variants' association with SLE or SjS and linkage with C4 variation (FIGS. 8A-8C).

Joint dosages of C4A and C4B for each individual in the same cohort were estimated by summing across their genotype probabilities of paired structural alleles that encode for the same diploid copy numbers of both C4A and C4B (FIGS. 6A and 6B). For each individual/genome, this yields a joint dosage distribution of C4A and C4B gene copy number, reflecting any possible imputed haplotype-level dosages with nonzero probability. Joint dosages for C4A and C4B diploid copy numbers were tested for association with SLE in a joint model with the same ancestry covariates (FIG. 1A),

logit(θ)˜β₀+Σ_{i,j}β_{i,j}P(C4A=i,C4B=j)+Σ_{c}β_{c}PC_{c}+ε (3)

### Calculation of Composite C4 Risk for SLE

Because SLE risk strongly associated with C4A and C4B copy numbers (FIG. 1A) in a manner that can be approximated as—but is not necessarily linear or independent—a composite C4 risk score was derived by taking the weighted sum of joint C4A and C4B dosages multiplied by the corresponding effect sizes from the aforementioned model of the joint C4A and C4B diploid copy numbers. The weights for calculating this composite C4 risk term were computed from the data from the European ancestry cohort, and then applied unchanged to analysis of the African American cohort.

### Associations of Variants Across the MHC Region to SLE and SjS

Genotypes for non-array SNPs were imputed with IMPUTE2 using the 1000 Genomes reference panel; separate analyses were performed for the European-ancestry and African American cohorts. Unless otherwise stated, all subsequent SLE analyses were performed identically for both European ancestry and African American cohorts. Dosage of each variant, vᵢ, was tested for association with SLE or SjS in a logistic regression including available ancestry covariates (and smoking status for SjS) first alone (FIG. 7),

logit(θ)˜β₀+β₁vᵢ+Σ_{c}β_{c}PC_{c}+ε (4)

then with C4 composite risk (FIG. 7),

logit(θ)˜β₀+β₁vᵢ+β₂C4+Σ_{c}β_{c}PC_{c}+ε (5)

and finally with C4 composite risk and rs2105898 dosage,

logit(θ)˜β₀+β₁vᵢ+β₂C4+β₃rs2105898+Σ_{c}β_{c}PC_{c}+ε (6)

where θ=E[SLE|X]. For SjS, the simpler weighted (2.3)C4A+C4B model was used instead of composite risk term, as the cohort's size gave poor precision to estimates of risk for many joint (C4A, C4B) copy numbers (FIG. 7). The Pearson correlation between the C4 composite risk term and each other variant was computed and squared (r2) to yield a measure of linkage disequilibrium between C4 composite risk and that variant in that cohort.

### Association Analyses for Specific C4 Structural Alleles

The C4 structural haplotypes were tested for association with disease (FIG. 1B and FIG. 2A) in a joint logistic regression that included (i) terms for dosages of the five most common C4 structural haplotypes (AL-BS, AL-BL, AL-AL, BS, and AL), (ii) (for SLE and SjS) rs2105898 genotype, and (iii) ancestry covariates and (for SjS) smoking status,

logit(θ)˜β₀+β₁BS+β₂AL+β₃ALBS+β₄ALBL+β₅ALAL+β₆rs2105898+Σ_{c}β_{c}PC_{c}+ε (7)

where θ=E[SLE|X]. Several of these common C4 structural alleles arose multiple times on distinct haplotypes; the set of haplotypes in which such a common allele appeared is termed “haplogroups”. The haplogroups can be further tested in a logistic regression model in which the structural allele appearing in all member haplotypes is instead encoded as dosages for each of the SNP haplotypes in which it appears.

These association analyses (FIG. 1B and FIG. 2A) were performed as in (6), with structural allele dosages for ALBS, ALBL, and ALAL replaced by multiple terms for each distinct haplotype. To delineate the relationship between C4-BS and DRB1*03:01 alleles—which are highly linked in European ancestry haplotypes—allelic dosages per individual in the African American SLE cohort were rounded to yield the most likely integer dosage for each. Although genotype dosages for each are reported by BEAGLE and HIBAG, respectively, probabilities per haplotype are not linked and multiplying possible diploid dosages could yield incorrect non-zero joint dosages. Joint genotypes were tested as individual terms in a logistic regression model (FIG. 2B),

logit(θ)˜β₀+Σ_{i,j}β_{i,j}P(C4-BS=i,DRB1*03:01=j)+Σ_{c}β_{c}PC_{c}+ε (8)

Sex-Stratified Associations of C4 Structural Alleles and Other Variants with SLE, SjS, and Schizophrenia

Determination of an effect from sex on the contribution of overall C4 variation to risk for each disorder was done by including an interaction term between sex and C4; i.e., (2.3)C4A+C4B for SLE and SjS and estimated C4A expression for schizophrenia:

logit(θ)˜β₀+β₂C4+β₃I_{Sex}+β₄I_{Sex}C4+Σ_{c}β_{c}PC_{c}+ε (9)

Each variant in the MHC region was tested for association with among European ancestry cases and cohorts in a logistic regression as in models (4)-(6) using only male cases and controls, and then separately using only female cases and controls (FIGS. 10A-10C). Likewise, allelic series analyses were performed as in (7), but in separate models for men and women (FIGS. 3A and 3B). To assess the relationship between sex bias in the risk associated with a variant and linkage to C4 composite risk (as non-negative r2), male and female log-odds were multiplied by the sign of the Pearson correlation between that variant and C4 composite risk before taking the difference.

### Analyses of Cerebrospinal Fluid

Cerebrospinal fluid (CSF) from healthy individuals was obtained from two research panels. The first panel consisted of 533 donors (327 male, 126 female) from hospitals around Utrecht, Netherlands. The donors were generally healthy research participants undergoing spinal anesthesia for minor elective surgery. The same donors were previously genotyped using the Illumina Omni SNP array. To estimate C4 copy numbers, SNPs from the MHC region (chr6:24-34 Mb on hg19) were used as input for C4 allele imputation with Beagle, as described hereinabove in “Imputation of C4 Alleles.”

The second CSF panel sampled specimens from 56 donors (14 male, 42 female) from Brigham and Women's Hospital (BWH; Boston, Mass., USA) under a protocol approved by the institutional review board at BWH (IRB protocol ID no. 1999P010911) with informed consent. These samples were originally obtained to exclude the possibility of infection, and clinical analyses had revealed no evidence of infection. Donors ranged in age from 18 to 64 years old. Blood samples from the same individuals were used for extraction of genomic DNA, and C4 gene copy number was measured by droplet digital PCR (ddPCR) as described, e.g., in Sekar, A. et al., 2016, Nature 530, 177-183.

Samples were excluded from measurements if they lacked C4 genotypes, sex information, or contained visible blood contamination. C4 measurements were performed by sandwich ELISA of 1:400 dilutions of the original CSF sample using goat anti-sera against human C4 as the capture antibody (Quidel, A305, used at 1:1000 dilution), FITCconjugated polyclonal rabbit anti-human C4c as the detection antibody (Dako, F016902-2, used at 1:3000 dilution), and alkaline phosphatase-conjugated polyclonal goat anti-rabbit IgG as the secondary antibody (Abcam, ab97048, used at 1:5000 dilution). C3 measurements were performed using the human complement C3 ELISA kit (Abcam, ab108823).

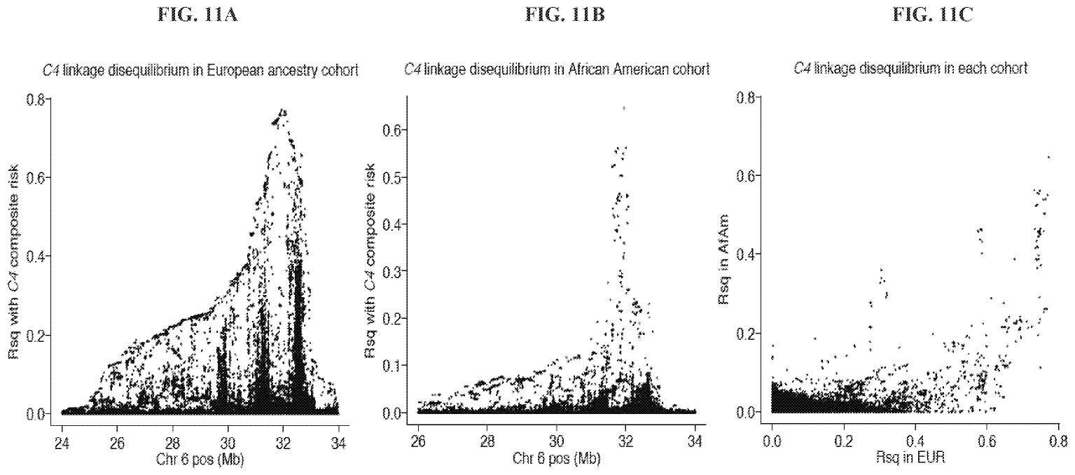

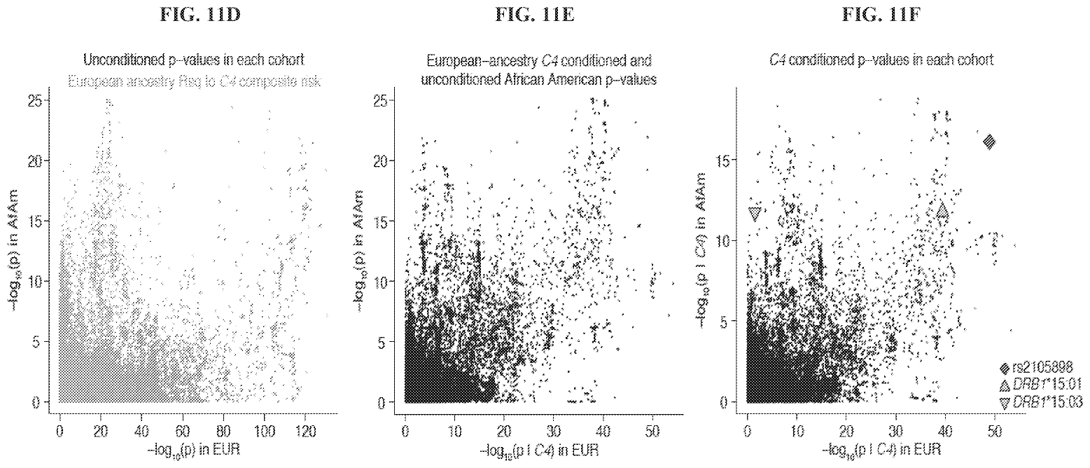

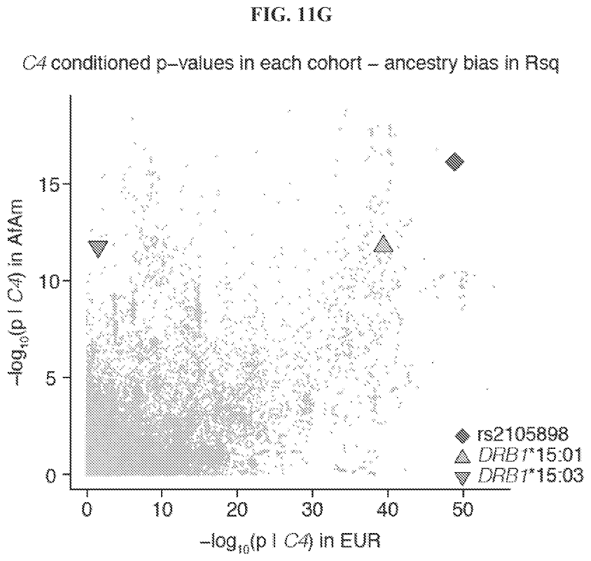

Because C4 gene copy number had a large and proportional effect on C4 protein concentration in these CSF samples (FIG. 11A), C4 gene copy number was corrected for in the analysis of relationship between sex and C4 protein concentration by normalizing the ratio of C4 protein (in CSF) to C4 gene copies (in genome). Therefore, these analyses included only samples for which DNA was available or C4 was successfully imputed. In total, 495 (332 male, 163 female) C4 and 304 (179 male, 125 female) C3 concentrations were obtained across both cohorts. Log-concentrations of C3 (ng/mL) and C4 (ng/[mL, per C4 gene copy number]) protein were then used separately in linear regression models to estimate a sex-unbiased cohort-specific offset for each protein,

log₁₀(C3 or C4 concentration)˜β₀+β₁Iₘₐₗₑ+β₂I_{cohort}+ε (10)

to be applied to all concentrations for that protein. Estimation of average measurements by age for each sex was done by local polynomial regression smoothing (LOESS) (FIGS. 3C and 3D). To evaluate the significance of sex effects, these cohort-corrected concentrations estimates were used and were analyzed with the nonparametric unsigned Mann-Whitney rank-sum test comparing concentration distributions for males and females.

### Analyses of Blood Plasma

Blood plasma was collected and immunoturbidimetric measurements of C3 and C4 protein in 1,844 individuals (182 men, 1662 women) were made by Sjögren's International Collaborative Clinical Alliance (SICCA) from individuals with and without SjS as described, e.g., in Malladi, A. S. et al., 2012, Arthritis Care Res (Hoboken) 64, 911-918, doi:10.1002/acr.21610. C4 copy numbers for these individuals were previously imputed for use in logistic regression of SjS risk. As C4 copy number has an effect on measured C4 protein similar to CSF (FIG. 11B), C4 levels were normalized to them in all following analyses. Estimation of average measurements by age for each sex was done by local polynomial regression smoothing (LOESS) on log-concentrations of C3 (mg/dL) and C4 (mg/[dL, per C4 gene copy number]) protein (FIGS. 11C and 11D). To evaluate the significance of sex bias within age ranges displaying the greatest difference (informed by LOESS), individuals in these bins were analyzed with the 20 non-parametric unsigned Mann-Whitney rank-sum test comparing concentration distributions for males and females. The difference in C4 protein levels between individual with and without SjS was done by performing a nonparametric unsigned Mann-Whitney rank-sum test on C4 protein levels with and without normalization to C4 genomic copy number (FIGS. 11E and 11F).

### Data Availability Statement

Individual genotype data for Sjögren's syndrome cases and controls and individual plasma concentrations for C4 and C3 are available in dbGaP under accession number phs000672.v1.p1. Individual genotype data for schizophrenia cases and controls are available by application to the Psychiatric Genomics Consortium (PGC).

### Example 3—Fine Mapping of an Independent Association Signal in the MHC Class II Region

### Linkage Disequilibrium of C4 Variation to Other MHC Variants Differs by Ancestry

The linkage-disequilibrium (LD) relationships of C4 variation to other genetic variation in the MHC locus differ greatly in magnitude and pattern between European-ancestry and African American cohorts. For example, FIGS. 11A and 11B show the LD-correlation (r²) of SNPs across the MHC locus to the composite estimate of C4-derived SLE risk employed in Examples 1 and 2 supra. (Other C4 features, such as total C4 gene copy number, also exhibit strikingly different correlations with genetic markers between the two populations). Most notably, LD in European ancestry is widespread across the extended MHC locus (FIG. 11A)—and particularly strong in the nearby MHC class II region (32-33 Mb)—while strong LD in African Americans is localized primarily to a much-smaller region immediately flanking the C4 genes (FIG. 11B).

A direct comparison of the two population-specific LD patterns confirms that nearly all variants with LD to C4 variation have greater LD in European-ancestry than in African American population sample, where only a small subset of European ancestry-linked alleles have similar or lower levels of linkage in African Americans (FIG. 11C).

### Initial (C4-naïve) Association Analysis Produces Divergent MHC Association Results in European-Ancestry and African American Cohorts

Unconditional (C4-naïve) association analysis of SLE of each variant in the MHC locus exhibits little correlation between European-ancestry and African American cohorts (FIG. 11D). Without wishing to be bound by theory, this may result from multiple population-specific variants or even population-specific biology. C4 alleles have both strong allele-frequency and LD differences between these populations (FIG. 13) and therefore could be a potential contributor to these differences in FIG. 11D. To appreciate this possibility, the points in FIG. 11D are presented in proportion to their European-ancestry LD (r²) to C4 composite risk. This highlights the strong effect that C4 alleles would be likely to have in shaping the relative association strengths of genetic markers throughout the MHC locus.

### Conditioning on C4 Composite Risk in European-Ancestry Cohort Only

Considering C4 in the above analysis provides an ability to align the association signals in Europeans and African Americans. If, beginning with the European-ancestry cohort, SNPs are considered not in a naïve association analysis, but in a joint association analysis together with C4 (i.e. with C4 genetic risk as a covariate), then the association statistics for variants in the two cohorts begin to align with each other more strongly (FIG. 11E).

Adjusting the association statistics for the African-American cohort analysis to account for C4 effects changed the overall pattern more modestly (FIG. 11F). Without intending to be bound by theory, this likely reflects reduced LD to C4 alleles among African Americans, and reduced C4 variation among African Americans relative to Europeans. (Population-specific HLA alleles (DRB1*15:01 and DRB1*15:03) have been proposed as potential explanations for the apparently divergent association signals across European ancestry and African American populations. In FIG. 11F, these variants are shown with grey triangles.)

Much of the population differences in SLE association pattern (that remain after controlling for C4) may be explained by differences in LD patterns between populations. In the same plots, coloring the variants by European or African American LD (r²) to rs2105898 reveals that the variants with higher relative associations in the European ancestry cohort (lower right in below plots) generally have higher LD to rs2105898 in that cohort. (This includes the European ancestry-specific SLE association to the HLA-DRB1*15:01 allele). Few variants have higher LD to rs2105898 in African Americans—though one such variant is the HLA-DRB1*15:03 allele, which has previously been reported to associate with SLE specifically in African Americans.

Much of the remaining differences in association pattern may be explained by differences in LD patterns between populations; in FIG. 11G, the areas denoted by the upright triangle represents greater LD to rs2105898 in the European-ancestry cohort (relative to LD among African Americans) and the areas denoted by the inverted triangle represent greater LD in the African American cohort (relative to LD among Europeans). Notably, the many variants with relatively stronger association signals among Europeans (including DRB1*1501) exhibit stronger LD to rs2105898 among Europeans, while select variants with relatively stronger association signals among African Americans (including DRB1*1503) exhibit stronger LD to rs2105898 among African Americans.

This analysis also indicates that while much, if not all, of the European ancestry-specific association, after controlling for C4 composite risk, can be accounted for by European ancestry-specific LD to rs2105898; this is not true for African Americans, who may harbor at least one additional, independent genetic effect not explained by the above analysis.

The C4-Independent Association Signal Comprising rs2105898 and Another Linked Variant Defines Strong Pan-Tissue Expression QTLs for HLA Class II Genes

Although rs2105898 was the top variant associated between cohorts in analyses controlling for C4, there is one other variant (rs9271513) in high (r2>0.9) LD across both populations that should be considered together as a haplotype. As mentioned in Example 1 supra, it was found that rs2105898 (and the highly LD-correlated variant) are significant eQTLs for 171 gene-tissue associations, largely comprised of significant associations for 7 HLA Class II genes (HLA-DRB1, HLA-DRB5, HLA-DRB6, HLA-DQA1, HLA-DQA2, HLA-DQB1, HLA-DQB2) in almost every tissue sampled by the GTEx Consortium.

The rs2105898 Haplotype Affects XL9 Hotspot of Active Chromatin and Transcription Factor Binding

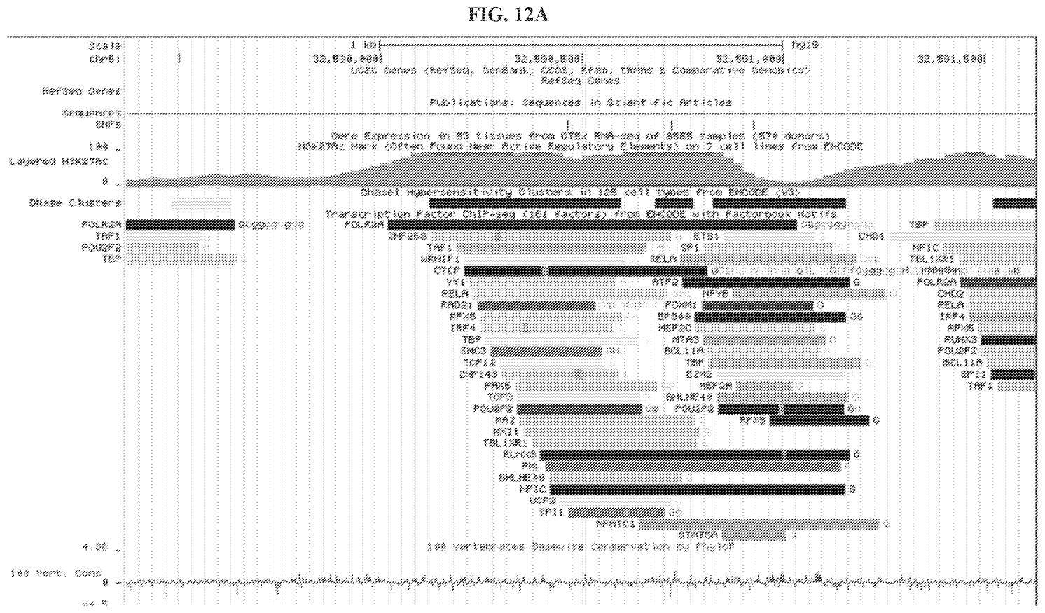

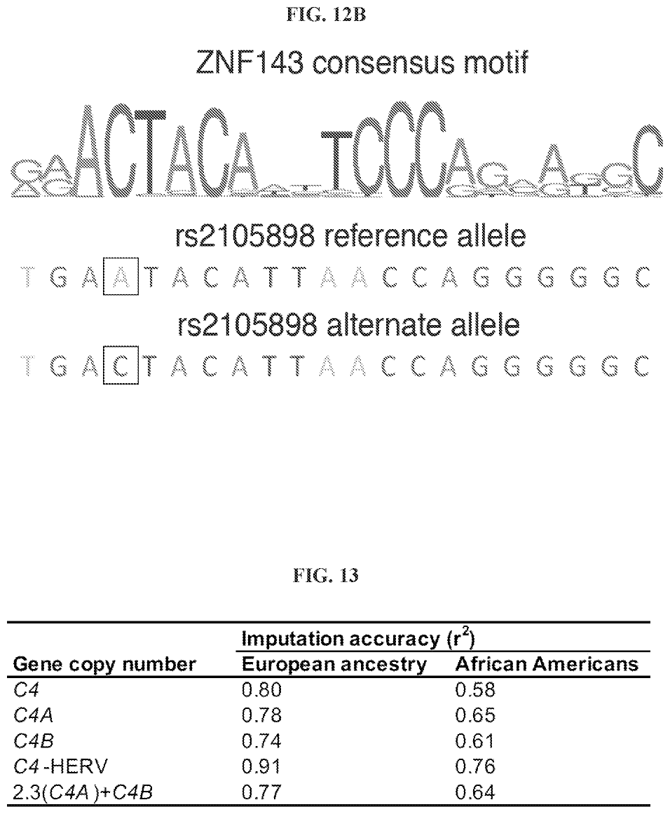

rs2105898 and the variant with which it is strong LD in both European and African American populations define a haplotype which is the effective unit of genetic association. rs2105898, in particular, lies within multiple histone marks that are associated with active enhancers (6 tissues), in the XL9 region of open chromatin (15 tissues), and under ChIP-seq binding peaks for 19 transcription factors (FIG. 12A, data from the ENCODE project (Center for Brain Science, Harvard University, Cambridge, Mass.).

rs2105898 Disrupts a Binding Site for the ZNF143 Transcription Factor

Transcription factors whose binding motif was significantly affected by rs2105898 allele were identified. The strongest hit (ZNF143) is also among the transcription factors that have been determined by ChIP-seq analysis (from the ENCODE project) to bind to DNA sequence at rs2105898 (FIG. 12B). ZNF143 is a widely expressed zinc-finger transcription factor that has been found to anchor chromatin interactions that connect distal regulatory elements with gene promoters.

Two databases (HaploReg, CIS-BP TF) evaluate ZNF143 as having low or no binding to the minor (reference) allele of rs2105898 and very high affinity to the major (alternate) allele of rs2105898:

CIS-BP (log score)
Reference (T) allele: 4.459
Alternate (G) allele: 13.273
HaploReg (log score)
Reference (T) allele: −0.4
Alternate (G) allele: 11.5

ZNF143 is a recently identified component of complexes that maintain topologically associated domains (TADs) in concert with CTCF and cohesin (SMC1, SMC3, RAD21, STAG1/2), both of which also have numerous ChIP-seq peaks overlapping rs2105898. Specifically, ZNF143 has been found to directly bind and regulate promoter interaction with distal enhancers, congruous with the observation of numerous RNA polymerase ChIP-seq peaks at rs2105898, but with the nearest promoter being 14.5 kb away (HLA-DQA1, downstream). Furthermore, as this region lies in the genomic neighborhood of many genes for which rs2105898 is a multi-tissue eQTL (HLA-DRB1, -DRB5, -DRB6 upstream and -DQA1, -DQA2, -DQB1, and -DQB2 downstream), it may be that by regulating ZNF143 binding, rs2105898 alters the interaction between this enhancer region and the promoters of the numerous proximal HLA class II genes.

rs2105898 is in Strong LD with Peak SNPs for Other Autoimmune Disorders

rs2105898 also has high LD to the most strongly associated SNPs for other autoimmune phenotypes. Of these associations, the strongest is to the peak SNP for multiple sclerosis oligoclonal band status (r²=0.88, D′=0.98). Also in high LD to rs2105898 is a shared peak SNP for associations to broad multiple sclerosis, immunoglobulin A production, ulcerative colitis, and Crohn's disease (all r²=0.49, D′=0.98).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for evaluating the propensity or risk of a subject for having or developing an autoimmune disease or disorder, the method comprising detecting in a sample obtained from the subject a dosage of C4A and C4B in the subject's genome, wherein increased dosage of C4A and C4B relative to a reference indicate that the subject has a reduced propensity or risk for having or developing the autoimmune disease or disorder.

2. The method of claim 1, wherein for each C4B copy number, a greater C4A copy number is associated with significantly reduced propensity or risk.

3. The method of claim 1, wherein for each C4A copy number, a greater C4B copy number is associated with more modestly reduced propensity or risk.

4. The method of claim 1, wherein the method further comprises calculating the subject's C4-derived risk score, wherein the risk score is calculated as 2.3 times the number of C4A genes, plus the number of C4B genes, in the subject's genome.

5. The method of claim 1, wherein the subject's joint C4A and C4B gene copy number is calculated by summing the C4A and C4B gene contents for each possible pair of two inherited C4 alleles.

6. The method of claim 5, wherein the C4 alleles are selected from the group consisting of B(S), A(L), A(L)-B(S)-2, A(L)-B(S)-3, A(L)-B(S)-4, A(L)-B(L)-1, A(L)-B(L)-2, A(L)-A(L)-1, A(L)-A(L)-2, and A(L)-A(L)-3.

7. The method of claim 1, wherein the protective effect of the C4A copy number is increased in a male subject relative to a female subject.

8. The method of claim 1, wherein the protective effect of the C4A copy number is increased in a subject of European ancestry relative to a subject of African ancestry.

9. The method of claim 1, wherein the autoimmune disease is systemic lupus erythematosus (SLE) or Sjögren's syndrome (SjS).

10. The method of claim 1, wherein the genome is characterized by whole genome sequencing.

11. The method of claim 1, wherein the sample comprises cells, plasma, or cerebral spinal fluid.

12. The method of claim 4, wherein calculating the subject's C4-derived risk score and/or joint C4A and C4B gene copy number is provided by performing computational analysis.

13. The method of claim 1, wherein computational analysis and/or an algorithm is applied for facilitating the determination of the subject's propensity or risk.

14. A method of treating inflammation in a subject, the method comprising administering an effective amount of a C4 inhibitor to the subject, thereby treating the inflammation.

15. The method of claim 14, wherein the inflammation is associated with a corona virus infection.

16. The method of claim 14, wherein the inflammation is associated with Covid19.

17. The method of claim 14, wherein the subject is a male.

18. The method of claim 17, wherein the effective amount of the C4 inhibitor is increased in a male subject relative to the amount of C4 inhibitor administered to the female subject.

19. The method of claim 14, wherein the C4 inhibitor is Eculizumab/Soliris, Cetor/Sanquin, or an anti-C1q antibody or fragment thereof.

20. A method of treating an autoimmune disorder in a subject, the method comprising administering an effective amount of a C4 agonist, activator, or C4 supplementing agent to the subject, thereby treating the autoimmune disorder.

21. The method of claim 20, wherein the autoimmune disorder is systemic lupus erythematosus (SLE) or Sjögren's syndrome (SjS).

22-23. (canceled)
